SEQUENCE LISTING (80 sequences)

SEQ ID NO: 1 - protein, 107 aa, Homo sapiens [sequence omitted]

SEQ ID NO: 2 - protein, 106 aa, synthetic construct
GQPKAAPSVTLFPPSSEELQANKATLVCFISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 106 aa, synthetic construct
GQPKAAPSVTLFPPSSEELQANKATLVCIISDFYPGAVTVAWKADSSPVKAGVETTTPSKQSNNKYAASSYLSLTPEQWKSHRSYSCQVTHEGSTVEKTVAPTECS

SEQ ID NO: 4 - protein, 106 aa, synthetic construct
GQPKAAPSVTLFPPSSEELQANKATLVCVISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 106 aa, synthetic construct
GQPKAAPSVTLFPPSSECLQANKATLVCFISDFYPGAVTVAWKADSSPVKAGVETTTPSKQSNNKYAASSYLSLTPEQWKSHRSYSCQVTHEGSTVEKTVAPTESS

SEQ ID NO: 6 - protein, 107 aa, Homo sapiens [sequence omitted]

SEQ ID NO: 7 - protein, 107 aa, synthetic construct
RTVAAPSVFIFPPSDEQLKSGTASVVCFLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 107 aa, synthetic construct
RTVAAPSVFIFPPSDECLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 9 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 10 - protein, 103 aa, synthetic construct
ASTKGPSVFPLAPSSKSTSGGTAALGCLVKDYFPEPVTVSWNSGALTSGVHTLPAVLQSSGLYSLISVVTVPSSSLGTQTYICNVNHKPSNTKVDKKVEPKSC

SEQ ID NO: 11 - protein, 98 aa, synthetic construct
ASTKGPSVFPLAPSSKSTSGGTAALGCLVKDYFPEPVTVSWNSGALTSGVHTIPAVLQSSGLYSLISVVTVPSSSLGTQTYICNVNHKPSNTKVDKKV

SEQ ID NO: 12 - protein, 103 aa, synthetic construct
ASTKGPSVFPLAPSSKSTSGGTAALGCLVKDYFPEPVTVSWNSGALTSGVHTIPAVLQSSGLYSLVSIVTVPSSSLGTQTYICNVNHKPSNTKVDKKVEPKSC

SEQ ID NO: 13 - protein, 103 aa, synthetic construct
ASTKGPSVFPLAPSSKSTSGGTAALGCLVKDYFPEPVTVSWNSGALTSGVHTLPAVLQSSGLYSLISLVTVPSSSLGTQTYICNVNHKPSNTKVDKKVEPKSC

SEQ ID NO: 14 - protein, 98 aa, synthetic construct
ASTKGPSVFPLAPSSKSTSGGTAALGCLVKDYFPEPVTVSWNSGALTSGVHTIPAVLQSSGLYSLVSLVTVPSSSLGTQTYICNVNHKPSNTKVDKKV

SEQ ID NO: 15 - protein, 103 aa, synthetic construct
ASTKGPSVFPLAPSSKSTSGGTAALGCLVKDYFPEPVTVSWNSGALTSGVHTIPAVLQSSGLYSLLSLVTVPSSSLGTQTYICNVNHKPSNTKVDKKVEPKSC

SEQ ID NO: 16 - protein, 103 aa, synthetic construct
ASTKGPSVFPLAPSSKSTSGGTAALGCLVKDYFPEPVTVSWNSGALTSGVHTLPAVLQSSGLYSLLSVVTVPSSSLGTQTYICNVNHKPSNTKVDKKVEPKSC

SEQ ID NO: 17 - protein, 103 aa, synthetic construct
ASTKGPSVFPLAPSSKSTSGGTAALGCLVKDYFPEPVTVSWNSGALTSGVHTLPAVLQSSGLYSLVSLVTVPSSSLGTQTYICNVNHKPSNTKVDKKVEPKSC

SEQ ID NO: 18 - protein, 103 aa, synthetic construct; identical to SEQ ID NO: 17

SEQ ID NO: 19 - protein, 103 aa, synthetic construct
ASTKGPSVFPLAPSSKSTSGGTAALGCLVKDYFPEPVTVSWNSGALTSGVHTVPAVLQSSGLYSLISLVTVPSSSLGTQTYICNVNHKPSNTKVDKKVEPKSC

SEQ ID NO: 20 - protein, 103 aa, synthetic construct
ASTKGPSVFPLAPSSKSTSGGTAALGCLVKDYFPEPVTVSWNSGALTSGVHTIPAVLQSSGLYSLVSVVTVPSSSLGTQTYICNVNHKPSNTKVDKKVEPKSC

SEQ ID NO: 21 - protein, 103 aa, synthetic construct
ASTKGPSVFPLAPSSKSTSGGTAALGCLVKDYFPEPVTVSWNSGALTSGVHTIPAVLQSSGLYSLLSVVTVPSSSLGTQTYICNVNHKPSNTKVDKKVEPKSC

SEQ ID NO: 22 - protein, 103 aa, synthetic construct
ASTKGPSVFPLAPSSKSTSGGTAALGCLVKDYFPEPVTVSWNSGALTSGVHTLPAVLQSSGLYSLVSIVTVPSSSLGTQTYICNVNHKPSNTKVDKKVEPKSC

SEQ ID NO: 23 - protein, 103 aa, synthetic construct
ASTKGPSVCPLAPSSKSTSGGTAALGCLVKDYFPEPVTVSWNSGALTSGVHTIPAVLQSSGLYSLLSLVTVPSSSLGTQTYICNVNHKPSNTKVDKKVEPKSS

SEQ ID NO: 24 - protein, 103 aa, synthetic construct
ASTKGPSVCPLAPSSKSTSGGTAALGCLVKDYFPEPVTVSWNSGALTSGVHTVPAVLQSSGLYSLISLVTVPSSSLGTQTYICNVNHKPSNTKVDKKVEPKSS

SEQ ID NO: 25 - protein, 448 aa, synthetic construct
  VARIANT 31..35: note = X = Any amino acid
  VARIANT 99..107: note = X= any amino acid
  VARIANT 50..66: note = X=any amino acid
QVQLVQSGAEVKKPGASVKVSCKASGYTFTXXXXXWVRQAPGQRLEWMGXXXXXXXXXXXXXXXXXRVTITVDMSASTAYMELSSLRSEDTAVYYCALXXXXXXXXXWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 26 - protein, 448 aa, synthetic construct
  VARIANT 31..35: note = X=any amino acid
  VARIANT 50..66: note = X=any amino acid
  VARIANT 99..107: note = X=any amino acid
QVQLVQSGAEVKKPGASVKVSCKASGYTFTXXXXXWVRQAPGQCLEWMGXXXXXXXXXXXXXXXXXRVTITVDMSASTAYMELSSLRSEDTAVYYCALXXXXXXXXXWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 27 - protein, 448 aa, synthetic construct
  VARIANT 31..35: note = X=any amino acid
  VARIANT 50..66: note = X=any amino acid
  VARIANT 99..107: note = X=any amino acid
QVQLVQSGAEVKKPGASVKVSCKASGYTFTXXXXXWVRQAPGQDLEWMGXXXXXXXXXXXXXXXXXRVTITVDMSASTAYMELSSLRSEDTAVYYCALXXXXXXXXXWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 28 - protein, 448 aa, synthetic construct
  VARIANT 31..35: note = X=any amino acid
  VARIANT 50..66: note = X=any amino acid
  VARIANT 99..107: note = X=any amino acid
QVQLVQSGAEVKKPGASVKVSCKASGYTFTXXXXXWVRQAPGQKLEWMGXXXXXXXXXXXXXXXXXRVTITVDMSASTAYMELSSLRSEDTAVYYCALXXXXXXXXXWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 29 - protein, 448 aa, synthetic construct
  VARIANT 31..35: note = X=any amino acid
  VARIANT 50..66: note = X=any amino acid
  VARIANT 99..107: note = X=any amino acid
QVQLVQSGAEVKKPGASVKVSCKASGYTFTXXXXXWVRDAPGQRLEWMGXXXXXXXXXXXXXXXXXRVTITVDMSASTAYMELSSLRSEDTAVYYCALXXXXXXXXXWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 30 - protein, 448 aa, synthetic construct
  VARIANT 31..35: note = X=any amino acid
  VARIANT 50..66: note = X=any amino acid
  VARIANT 99..107: note = X=any amino acid
QVQLVQSGAEVKKPGASVKVSCKASGYTFTXXXXXWVRKAPGQRLEWMGXXXXXXXXXXXXXXXXXRVTITVDMSASTAYMELSSLRSEDTAVYYCALXXXXXXXXXWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 31 - protein, 452 aa, synthetic construct
  VARIANT 31..35: note = X=any amino acid
  VARIANT 50..66: note = X=any amino acid
  VARIANT 99..107: note = X=any amino acid
QVQLVQSGAEVKKPGSSVKVSCKASGYTFTXXXXXWVRQAPGQGLEWMGXXXXXXXXXXXXXXXXXRVTITVDTSTSTAYMELSSLRSEDTAVYYCARXXXXXXXXXXXXXWGQGTLVTVSSASTKGPSVFPLAPSSKSTSGGTAALGCLVKDYFPEPVTVSWNSGALTSGVHTVPAVLQSSGLYSLISLVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 32 - protein, 452 aa, synthetic construct
  VARIANT 31..35: note = X=any amino acid
  VARIANT 50..66: note = X=any amino acid
  VARIANT 99..107: note = X=any amino acid
QVQLVQSGAEVKKPGSSVKVSCKASGYTFTXXXXXWVRQAPGQCLEWMGXXXXXXXXXXXXXXXXXRVTITVDTSTSTAYMELSSLRSEDTAVYYCARXXXXXXXXXXXXXWGQGTLVTVSSASTKGPSVFPLAPSSKSTSGGTAALGCLVKDYFPEPVTVSWNSGALTSGVHTVPAVLQSSGLYSLISLVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 33 - protein, 452 aa, synthetic construct
  VARIANT 31..35: note = X=any amino acid
  VARIANT 50..66: note = X=any amino acid
  VARIANT 99..107: note = X=any amino acid
QVQLVQSGAEVKKPGSSVKVSCKASGYTFTXXXXXWVRQAPGQDLEWMGXXXXXXXXXXXXXXXXXRVTITVDTSTSTAYMELSSLRSEDTAVYYCARXXXXXXXXXXXXXWGQGTLVTVSSASTKGPSVFPLAPSSKSTSGGTAALGCLVKDYFPEPVTVSWNSGALTSGVHTVPAVLQSSGLYSLISLVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 34 - protein, 452 aa, synthetic construct
  VARIANT 31..35: note = X=any amino acid
  VARIANT 50..66: note = X=any amino acid
  VARIANT 99..107: note = X=any amino acid
QVQLVQSGAEVKKPGSSVKVSCKASGYTFTXXXXXWVRQAPGQKLEWMGXXXXXXXXXXXXXXXXXRVTITVDTSTSTAYMELSSLRSEDTAVYYCARXXXXXXXXXXXXXWGQGTLVTVSSASTKGPSVFPLAPSSKSTSGGTAALGCLVKDYFPEPVTVSWNSGALTSGVHTVPAVLQSSGLYSLISLVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 35 - protein, 452 aa, synthetic construct
  VARIANT 31..35: note = X=any amino acid
  VARIANT 50..66: note = X=any amino acid
  VARIANT 89..97: note = X=any amino acid
QVQLVQSGAEVKKPGSSVKVSCKASGYTFTXXXXXWVRDAPGQGLEWMGXXXXXXXXXXXXXXXXXRVTITVDTSTSTAYMELSSLRSEDTAVYYCARXXXXXXXXXXXXXWGQGTLVTVSSASTKGPSVFPLAPSSKSTSGGTAALGCLVKDYFPEPVTVSWNSGALTSGVHTVPAVLQSSGLYSLISLVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 36 - protein, 452 aa, synthetic construct
  VARIANT 31..35: note = X=any amino acid
  VARIANT 50..66: note = X=any amino acid
  VARIANT 99..107: note = X=any amino acid
QVQLVQSGAEVKKPGSSVKVSCKASGYTFTXXXXXWVRKAPGQGLEWMGXXXXXXXXXXXXXXXXXRVTITVDTSTSTAYMELSSLRSEDTAVYYCARXXXXXXXXXXXXXWGQGTLVTVSSASTKGPSVFPLAPSSKSTSGGTAALGCLVKDYFPEPVTVSWNSGALTSGVHTVPAVLQSSGLYSLISLVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 37 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQQKPGKAPKLLIYXXXXXXXGVPSRFSGSGSGTEFTLTISSLQPEDFATYYCXXXXXXXXXFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQQKPGKAPKLLIYXXXXXXXGVPSRFSGSGSGTEFTLTISSLQPEDFATYYCXXXXXXXXXFGCGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQQKPGKAPKLLIYXXXXXXXGVPSRFSGSGSGTEFTLTISSLQPEDFATYYCXXXXXXXXXFGDGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQQKPGKAPKLLIYXXXXXXXGVPSRFSGSGSGTEFTLTISSLQPEDFATYYCXXXXXXXXXFGKGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQQKPGKAPKLLIYXXXXXXXGVPSRFSGSGSGTEFTLTISSLQPEDFATYYCXXXXXXXXXFGQDTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQQKPGKAPKLLIYXXXXXXXGVPSRFSGSGSGTEFTLTISSLQPEDFATYYCXXXXXXXXXFGQKTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQDKPGKAPKLLIYXXXXXXXGVPSRFSGSGSGTEFTLTISSLQPEDFATYYCXXXXXXXXXFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQKKPGKAPKLLIYXXXXXXXGVPSRFSGSGSGTEFTLTISSLQPEDFATYYCXXXXXXXXXFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQQKPGKAPKLLIYXXXXXXXGVPSRFSGSGSGTDFTLTISSLQPEDFATYYCXXXXXXXXXFGGGTKVEIKRTVAAPSVFIFPPSDEQLKSGTASVVCFLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQQKPGKAPKLLIYXXXXXXXGVPSRFSGSGSGTDFTLTISSLQPEDFATYYCXXXXXXXXXFGCGTKVEIKRTVAAPSVFIFPPSDEQLKSGTASVVCFLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQQKPGKAPKLLIYXXXXXXXGVPSRFSGSGSGTDFTLTISSLQPEDFATYYCXXXXXXXXXFGDGTKVEIKRTVAAPSVFIFPPSDEQLKSGTASVVCFLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQQKPGKAPKLLIYXXXXXXXGVPSRFSGSGSGTDFTLTISSLQPEDFATYYCXXXXXXXXXFGKGTKVEIKRTVAAPSVFIFPPSDEQLKSGTASVVCFLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQQKPGKAPKLLIYXXXXXXXGVPSRFSGSGSGTDFTLTISSLQPEDFATYYCXXXXXXXXXFGGDTKVEIKRTVAAPSVFIFPPSDEQLKSGTASVVCFLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQQKPGKAPKLLIYXXXXXXXGVPSRFSGSGSGTDFTLTISSLQPEDFATYYCXXXXXXXXXFGGKTKVEIKRTVAAPSVFIFPPSDEQLKSGTASVVCFLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQDKPGKAPKLLIYXXXXXXXGVPSRFSGSGSGTDFTLTISSLQPEDFATYYCXXXXXXXXXFGGGTKVEIKRTVAAPSVFIFPPSDEQLKSGTASVVCFLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 214 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQKKPGKAPKLLIYXXXXXXXGVPSRFSGSGSGTDFTLTISSLQPEDFATYYCXXXXXXXXXFGGGTKVEIKRTVAAPSVFIFPPSDEQLKSGTASVVCFLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 118 aa, synthetic construct
  VARIANT 31..35: note = X=any amino acid
  VARIANT 50..66: note = X=any amino acid
  VARIANT 99..107: note = X=any amino acid
QVQLVQSGAEVKKPGASVKVSCKASGYTFTXXXXXWVRQAPGQRLEWMGXXXXXXXXXXXXXXXXXRVTITVDMSASTAYMELSSLRSEDTAVYYCALXXXXXXXXXWGQGTLVTVSS

SEQ ID NO: 54 - protein, 118 aa, synthetic construct
  VARIANT 31..35: note = X=any amino acid
  VARIANT 50..66: note = X=any amino acid
  VARIANT 99..107: note = X=any amino acid
QVQLVQSGAEVKKPGASVKVSCKASGYTFTXXXXXWVRQAPGQCLEWMGXXXXXXXXXXXXXXXXXRVTITVDMSASTAYMELSSLRSEDTAVYYCALXXXXXXXXXWGQGTLVTVSS

SEQ ID NO: 55 - protein, 118 aa, synthetic construct
  VARIANT 31..35: note = X=any amino acid
  VARIANT 50..66: note = X=any amino acid
  VARIANT 99..107: note = X=any amino acid
QVQLVQSGAEVKKPGASVKVSCKASGYTFTXXXXXWVRQAPGQDLEWMGXXXXXXXXXXXXXXXXXRVTITVDMSASTAYMELSSLRSEDTAVYYCALXXXXXXXXXWGQGTLVTVSS

SEQ ID NO: 56 - protein, 118 aa, synthetic construct
  VARIANT 31..35: note = X=any amino acid
  VARIANT 50..66: note = X=any amino acid
  VARIANT 99..107: note = X=any amino acid
QVQLVQSGAEVKKPGASVKVSCKASGYTFTXXXXXWVRQAPGQKLEWMGXXXXXXXXXXXXXXXXXRVTITVDMSASTAYMELSSLRSEDTAVYYCALXXXXXXXXXWGQGTLVTVSS

SEQ ID NO: 57 - protein, 118 aa, synthetic construct
  VARIANT 31..35: note = X=any amino acid
  VARIANT 50..66: note = X=any amino acid
  VARIANT 99..107: note = X=any amino acid
QVQLVQSGAEVKKPGASVKVSCKASGYTFTXXXXXWVRDAPGQRLEWMGXXXXXXXXXXXXXXXXXRVTITVDMSASTAYMELSSLRSEDTAVYYCALXXXXXXXXXWGQGTLVTVSS

SEQ ID NO: 58 - protein, 118 aa, synthetic construct
  VARIANT 31..35: note = X=any amino acid
  VARIANT 50..66: note = X=any amino acid
  VARIANT 99..107: note = X=any amino acid
QVQLVQSGAEVKKPGASVKVSCKASGYTFTXXXXXWVRKAPGQRLEWMGXXXXXXXXXXXXXXXXXRVTITVDMSASTAYMELSSLRSEDTAVYYCALXXXXXXXXXWGQGTLVTVSS

SEQ ID NO: 59 - protein, 122 aa, synthetic construct
  VARIANT 31..35: note = X=any amino acid
  VARIANT 50..66: note = X=any amino acid
  VARIANT 99..107: note = X=any amino acid
QVQLVQSGAEVKKPGSSVKVSCKASGYTFTXXXXXWVRQAPGQGLEWMGXXXXXXXXXXXXXXXXXRVTITVDTSTSTAYMELSSLRSEDTAVYYCARXXXXXXXXXXXXXWGQGTLVTVSS

SEQ ID NO: 60 - protein, 122 aa, synthetic construct
  VARIANT 31..35: note = X=any amino acid
  VARIANT 50..66: note = X=any amino acid
  VARIANT 99..107: note = X=any amino acid
QVQLVQSGAEVKKPGSSVKVSCKASGYTFTXXXXXWVRQAPGQCLEWMGXXXXXXXXXXXXXXXXXRVTITVDTSTSTAYMELSSLRSEDTAVYYCARXXXXXXXXXXXXXWGQGTLVTVSS

SEQ ID NO: 61 - protein, 122 aa, synthetic construct
  VARIANT 31..35: note = X=any amino acid
  VARIANT 50..66: note = X=any amino acid
  VARIANT 99..107: note = X=any amino acid
QVQLVQSGAEVKKPGSSVKVSCKASGYTFTXXXXXWVRQAPGQDLEWMGXXXXXXXXXXXXXXXXXRVTITVDTSTSTAYMELSSLRSEDTAVYYCARXXXXXXXXXXXXXWGQGTLVTVSS

SEQ ID NO: 62 - protein, 122 aa, synthetic construct
  VARIANT 31..35: note = X=any amino acid
  VARIANT 50..66: note = X=any amino acid
  VARIANT 99..107
QVQLVQSGAEVKKPGSSVKVSCKASGYTFTXXXXXWVRQAPGQKLEWMGXXXXXXXXXXXXXXXXXRVTITVDTSTSTAYMELSSLRSEDTAVYYCARXXXXXXXXXXXXXWGQGTLVTVSS

SEQ ID NO: 63 - protein, 122 aa, synthetic construct
  VARIANT 31..35: note = X=any amino acid
  VARIANT 50..66: note = X=any amino acid
  VARIANT 99..107: note = X=any amino acid
QVQLVQSGAEVKKPGSSVKVSCKASGYTFTXXXXXWVRDAPGQGLEWMGXXXXXXXXXXXXXXXXXRVTITVDTSTSTAYMELSSLRSEDTAVYYCARXXXXXXXXXXXXXWGQGTLVTVSS

SEQ ID NO: 64 - protein, 122 aa, synthetic construct
  VARIANT 31..35: note = X=any amino acid
  VARIANT 50..66: note = X=any amino acid
  VARIANT 99..107: note = X=any amino acid
QVQLVQSGAEVKKPGSSVKVSCKASGYTFTXXXXXWVRKAPGQGLEWMGXXXXXXXXXXXXXXXXXRVTITVDTSTSTAYMELSSLRSEDTAVYYCARXXXXXXXXXXXXXWGQGTLVTVSS

SEQ ID NO: 65 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQQKPGKAPKLLIYXXXXXXXGVPSRFSGSGSGTEFTLTISSLQPEDFATYYCXXXXXXXXXFGQGTKLEIK

SEQ ID NO: 66 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQQKPGKAPKLLIYXXXXXXXGVPSRFSGSGSGTEFTLTISSLQPEDFATYYCXXXXXXXXXFGCGTKLEIK

SEQ ID NO: 67 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQQKPGKAPKLLIYXXXXXXXGVPSRFSGSGSGTEFTLTISSLQPEDFATYYCXXXXXXXXXFGDGTKLEIK

SEQ ID NO: 68 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQQKPGKAPKLLIYXXXXXXXGVPSRFSGSGSGTEFTLTISSLQPEDFATYYCXXXXXXXXXFGKGTKLEIK

SEQ ID NO: 69 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQQKPGKAPKLLIYXXXXXXXGVPSRFSGSGSGTEFTLTISSLQPEDFATYYCXXXXXXXXXFGQDTKLEIK

SEQ ID NO: 70 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQQKPGKAPKLLIYXXXXXXXGVPSRFSGSGSGTEFTLTISSLQPEDFATYYCXXXXXXXXXFGQKTKLEIK

SEQ ID NO: 71 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQDKPGKAPKLLIYXXXXXXXGVPSRFSGSGSGTEFTLTISSLQPEDFATYYCXXXXXXXXXFGQGTKLEIK

SEQ ID NO: 72 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSFLSASVGDRVTITCXXXXXXXXXXXWYQKKPGKAPKLLIYXXXXXXXGVPSRFSGSGSGTEFTLTISSLQPEDFATYYCXXXXXXXXXFGQGTKLEIK

SEQ ID NO: 73 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQQKPGKAPKLLIYXXXXXXXGVPSRFSGSGSGTDFTLTISSLQPEDFATYYCXXXXXXXXXFGGGTKVEIK

SEQ ID NO: 74 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQQKPGKAPKLLIYXXXXXXXGVPSRFSGSGSGTDFTLTISSLQPEDFATYYCXXXXXXXXXFGCGTKVEIK

SEQ ID NO: 75 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQQKPGKAPKLLIYXXXXXXXGVPSRFSGSGSGTDFTLTISSLQPEDFATYYCXXXXXXXXXFGDGTKVEIK

SEQ ID NO: 76 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQQKPGKAPKLLIYXXXXXXXGVPSRFSGSGSGTDFTLTISSLQPEDFATYYCXXXXXXXXXFGKGTKVEIK

SEQ ID NO: 77 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQQKPGKAPKLLIYXXXXXXXGVPSRFSGSGSGTDFTLTISSLQPEDFATYYCXXXXXXXXXFGGDTKVEIK

SEQ ID NO: 78 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQQKPGKAPKLLIYXXXXXXXGVPSRFSGSGSGTDFTLTISSLQPEDFATYYCXXXXXXXXXFGGKTKVEIK

SEQ ID NO: 79 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQDKPGKAPKLLIYXXXXXXXGVPSRFSGSGSGTDFTLTISSLQPEDFATYYCXXXXXXXXXFGGGTKVEIK

SEQ ID NO: 80 - protein, 107 aa, synthetic construct
  VARIANT 24..34: note = X=any amino acid
  VARIANT 50..56: note = X=any amino acid
  VARIANT 89..97: note = X=any amino acid
DIQMTQSPSSLSASVGDRVTITCXXXXXXXXXXXWYQKKPGKAPKLLIYXXXXXXXGVPSRFSGSGSGTDFTLTISSLQPEDFATYYCXXXXXXXXXFGGGTKVEIK

(19) United States
(12) Patent Application Publication
(10) Pub. No.: US 2026/0250377 A1
(43) Pub. Date: Aug. 27, 2026

(54) ANTIBODIES AND METHODS OF PRODUCING ANTIBODIES

(71) Applicant: Proteologix US Inc., Redwood City, CA (US)

(72) Inventors: Jie Tang, Redwood City, CA (US); Danming Tang, Redwood City, CA (US); Minghui Xu, Redwood City, CA (US)

(21) Appl. No.: 19/489,660

(22) PCT Filed: Jun. 4, 2024
(86) PCT No.: PCT/US2024/032454

§ 371 Date: Dec. 3, 2025

Related U.S. Application Data
(60) Provisional application No. 63/506,301, filed on Jun. 5, 2023.

(51) Int. Cl. C07K 16/26; C07K 16/28

(52) U.S. Cl. CPC C07K 16/26; C07K 16/2866; C07K 2317/31; C07K 2317/55

(57) ABSTRACT

The present disclosure generally relates to antibodies and methods of producing antibodies. Specifically, the present disclosure relates to antibodies and antigen-binding fragments thereof having a VH1, VH2, VL1 and VL2, and modifications which assist in increasing the desired pairing of VH to VL, for example VH1/VL1 and VH2/VL2 rather than VH1/VL2 and VH2/VL1.

### INCORPORATION BY REFERENCE TO ANY PRIORITY APPLICATIONS

The present application claims priority to U.S. Provisional Application 63/506,301, filed on Jun. 5, 2023, which is herein incorporated by reference in its entirety.

### REFERENCE TO SEQUENCE LISTING

The present application is being filed along with a Sequence Listing in electronic format. The Sequence Listing is provided as a file entitled PROGX.001WOSEQLISTEQIST.XML, which was created and last modified on May 24, 2024 which is 147,428 bytes in size. The information in the electronic Sequence Listing is hereby incorporated by reference in its entirety.

### FIELD

The present disclosure generally relates to antibodies and methods of producing antibodies.

### BACKGROUND

Multi-specific antibodies have shown to be a promising strategy to overcome some of the fundamental limitations of monospecific antibody therapies, which cannot address the multiple cross-talking signaling pathways underlying the targeted diseases. Although efforts have been taken to address the immunogenicity, chain mispairing issues and manufacturing scalability associated with multivalent molecules, there remains a need for the development of a multi-specific format that effectively addresses these issues; Brinkmann U. Kontermann R E. 2017. “The making of bispecific antibodies.” mAbs 9:182-212; Godar et Godar M, de Haard H, Blanchetot C, Rasser J. 2018. “Therapeutic bispecific antibody formats: a patent applications review” (1994-2017). Expert. Opin. Ther. Pat. 28:251-276.

### SUMMARY

Some embodiments provided herein are described by way of the following numbered embodiments and also provided as possible combinations or overlapping embodiments:
1. An antigen-binding fragment (Fab) comprising:
a heavy chain (HC) variable domain (VH), a heavy chain (HC) constant domain (CH1) of a human IgG a light chain (LC) variable domain (VL), and a light chain (LC) constant domain (CL) of a human IgG, wherein the HC CH1 comprises a substitution at F170, S183, and V185 (EU Numbering), and the LC CL comprises a substitution at L135 (EU Numbering), wherein the HC1 does not include any substitution selected from L128F, A141M, A141T, A141I, F170M, F170Y, F170S, F170A, S181I, S181T, S181M, S183A, S183V, and V185A (EU Numbering), and wherein the CL does not include any substitution selected from F116A, F118V, S131T, S131D, V133A, V133I, L13SY, L135V, S162A, S162M, T164S, S174A, S176M, S176A, S176T, S176F, T178L, T178V, and T178I (European Numbering).
2. The Fab of embodiment 1, wherein the CH1 consists of substitutions at F170, S183 and V185 (EU Numbering), and wherein the CL consists of a substitution at L135 (EU Numbering).
3. The Fab of any one of embodiments 1-2, wherein the HC CH1 substitution at F170 (EU Numbering) is an F170I, or F170V substitution.
4. The Fab of any one of embodiments 1-3, wherein the HC CH1 substitution at S183 (EU Numbering) is a S183L or S1831 substitution.
5. The Fab of any one of embodiments 1-4, wherein the HC CH1 substitution at V185 (EU Numbering) is a V185L substitution.
6. The Fab of any one of embodiments 1-5, wherein the LC CL1 substitution at L135 (EU Numbering) is a L135F substitution.
7. The Fab of embodiment 1 or 2, wherein the HC CH1 comprises or consists of an F170V, S1831 and V185L (EU Numbering) substitution, and the LC CL comprises or consists of a L135F (EU Numbering) substitution.
8. The Fab of embodiment 1 or 2, wherein the HC CH1 comprises or consists of an F170I, S183L, and V185L (EU Numbering) substitution, and the LC CL comprises or consists of a L135F (EU Numbering) substitution.
9 An antigen-binding fragment (Fab) comprising:
a heavy chain (HC) variable domain (VH), a heavy chain (HC) constant domain (CH1) of a human IgG, a light chain (LC) variable domain (VL), and a light chain (LC) constant domain (CL) of a human IgG, wherein the HC CH1 comprises a substitution at F126, F170, S183, V185, and C220 (EU Numbering), and the LC CL comprises a substitution at E124, L135, and C214 (lambda) or Q124, L135, and C214 (kappa) (EU Numbering), optionally wherein the LC CL comprises a substitution at E124, L135, and C214 (lambda), or optionally wherein the LC CL comprises a substitution at Q124, L135, and C214 (kappa), wherein the HC1 does not include any substitution selected from L128F, A141M, A141T, A141I, F170M, F170Y, F170S, F170A, S181I, S181T, S181M, S183A, S183V, and V185A (EU Numbering), and wherein the CL does not include any substitution selected from F116A, F118V, S13IT, S131D, V133A, V133I, L135Y, L135V, S162A, S162M, T164S, S174A, S176M, S176A, S176T, S176F, T178L, T178V, and T178I (EU Numbering).
10. The Fab of embodiment 9, wherein the CH1 consists of substitutions at F126, F170, S183, V185, and C220 (EU Numbering), and the LC CL comprises a substitution at E124, L135, and C214 (lambda) or Q124, L135, and C214 (kappa) (EU Numbering), optionally wherein the LC CL comprises a substitution at E124, L135, and C214 (lambda), or optionally wherein the LC CL comprises a substitution at Q124, L135, and C214 (kappa).
11. The Fab of embodiment 9 or 10, wherein the HC CH1 substitution at F126 (EU Numbering) is an F126C substitution.
12. The Fab of any one of embodiments 9-11, wherein the HC CH1 substitution at F170 (EU Numbering) is an F170V or F170I substitution.
13. The Fab of any one of embodiments 9-12, wherein the HC CH1 substitution at S183 (EU Numbering) is a S183L, or S183I substitution.
14. The Fab of any one of embodiments 9-13, wherein the HC CH1 substitution at V185 (EU Numbering) is a V185L substitution.
15. The Fab of any one of embodiments 9-14, wherein the HC CH1 substitution at C220 (EU Numbering) is a C220S substitution.
16. The Fab of any one of embodiments 9-15, wherein the LC CL substitution at E124 (lambda) or Q124 (kappa) (EU Numbering) is an E124C or Q124C substitution, optionally wherein the LC CL substitution at E124 (lambda) is an E124C substitution, or optionally wherein the LC CL substitution at Q124 (kappa) is a Q124C substitution.
17. The Fab of any one of embodiments 9-16, wherein the LC CL substitution at L135 (EU Numbering) is a L135F substitution.
18. The Fab of any one of embodiments 9-17, wherein the LC CL substitution at C214 (EU Numbering) is a C214S substitution.
19. The Fab of embodiment 9, wherein the HC CH1 comprises or consists of an F126C, F170V, S183I, V185L, and C220S (EU Numbering) substitution, and the LC CL comprises an E124C, L135F, and C214S (lambda) or Q124C, L135F, and C214S (kappa) (EU Numbering) substitution, optionally wherein the LC CL comprises an E124C, L135F, and C214S (lambda) (EU Numbering) substitution, or optionally wherein the LC CL comprises a Q124C, L135F, and C214S (kappa) (EU Numbering) substitution.
20. The Fab of embodiment 9, wherein the HC CH1 comprises or consists of an F126C, F1701, S183L, V185L, and C220S (EU Numbering) substitution, and the LC CL comprises an E124C, L135F, and C214S (lambda) or Q124, L135, and C214 (kappa) (EU Numbering) substitution, optionally wherein the LC CL comprises an E124C, L135F, and C214S (lambda) (EU Numbering) substitution, or optionally wherein the LC CL comprises a Q124, L135, and C214 (kappa) (EU Numbering) substitution.
21. An antigen-binding fragment (Fab) comprising:
a heavy chain (HC) variable domain (VH), a heavy chain (HC) constant domain (CH1) of a human IgG a light chain (LC) variable domain (VL), and a light chain (LC) constant domain (CL) of a human IgG, wherein the HC CH1 comprises a substitution at F126 and C220 (EU Numbering), and the LC CL comprises a substitution at E124 and C214 (lambda) or Q124 and C214 (kappa) (EU Numbering), optionally wherein the LC CL comprises a substitution at E124 and C214 (lambda) (EU Numbering), or optionally wherein the LC CL comprises a substitution at Q124 and C214 (kappa) (EU Numbering);
wherein the HC1 does not include any substitution selected from L128F, A141M, A14IT, A141I, F170I, F170M, F170Y, F170S, F170A, F170V, S181I, S181T, S18IM, S183A, S183L, S183I, S183V, V185L and V185A (EU Numbering);
   and wherein the CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (European Numbering).
22. The Fab of embodiment 21, wherein the HC CH1 substitution at F126 (EU Numbering) is an F126C substitution, and the LC CL substitution at position 124 (EU Numbering) is an E124C (lambda) or Q124C (kappa) substitution, optionally wherein the LC CL substitution at position 124 (EU Numbering) is an E124C (lambda) substitution, or optionally wherein the LC CL substitution at position 124 (EU Numbering) is a Q124C (kappa) substitution.
23. The Fab of any one of embodiments 21-22, wherein the HC CH1 substitution at C220 (EU Numbering) is a C220S substitution.
24. The Fab of any one of embodiments 21-23, wherein the LC CL substitution at C214 (EU Numbering) is a C214S substitution.
25. The Fab of any one of embodiments 1-24, wherein the HC CH1 comprises or consists of SEQ ID NO: 15 or 19, and the LC CL comprises or consists of SEQ ID NO: 2 or 7; or wherein the HC CH1 comprises or consists of SEQ ID NO: 23 or 24, and the LC CL comprises or consists of SEQ ID NO: 5 or 8.
26. An antibody comprising the Fab of any one of embodiments 1-25.
27. The antibody of embodiment 26, further comprising a second Fab, wherein one but not both of the Fabs comprise an HC CH1 comprising a substitution at F126 and C220 (EU Numbering), and a LC CL comprising a substitution at E124 and C214 (lambda) or Q124 and C214 (kappa) (EU Numbering), optionally wherein a LC CL comprising a substitution at E124 and C214 (lambda) (EU Numbering), or optionally a LC CL comprising a substitution at Q124 and C214 (kappa) (EU Numbering).
28. The antibody of embodiment 27, wherein the HC CH1 substitution at F126 (EU Numbering) is an F126C substitution, and the LC CL substitution at position 124 (EU Numbering) is an E124C (lambda) or Q124C (kappa) substitution, optionally wherein the LC CL substitution at position 124 (EU Numbering) is an E124C (lambda) substitution, or optionally the LC CL substitution at position 124 (EU Numbering) is a Q124C (kappa) substitution.
29. The antibody of any one of embodiments 27-28, wherein the HC CH1 substitution at C220 (EU Numbering) is a C220S substitution.
30. The antibody of any one of embodiments 27-29, wherein the LC CL substitution at C214 (EU Numbering) is a C214S substitution.
31. An antibody comprising one or more IgG Fabs having a wild-type CH and a wild-type CL, and one or more Fabs of any one of embodiments 1-30.
32. The antibody of any one of embodiments 27-31, wherein the antibody comprises the Fab of any one of embodiments 1-8 and the Fab of any one of embodiments 21-24, or the Fab of any one of embodiments 9-20 and a Fab not comprising a HC CH1 comprising a substitution at F126 and C220 (EU Numbering), and a LC CL comprising a substitution at position 124 (E124 for lambda and Q124 for kappa) and C214 (EU Numbering), optionally wherein a LC CL comprising a substitution at position E124 for lambda and C214 (EU Numbering), or optionally wherein a LC CL comprising a substitution at position Q124 for kappa and C214 (EU Numbering).
33. An antibody or a fragment thereof comprising:
a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG,
wherein the HC1 CH1 comprises a substitution at F170, S183, and V185 (EU Numbering), and the LC1 CL comprises a substitution at L135 (EU Numbering), wherein the HC1 CH1 does not include any substitution selected from L128F, A141M, A14IT, A141I, F170M, F170Y, F170S, F170A, S181I, S181T, S18IM, S183A, S183V, and V185A (EU Numbering),
and wherein the LC1 CL does not include any substitution selected from F116A, F118V, S13IT, S131D, V133A, V133I, L135Y, L13SV, S162A, S162M, T164S, S174A, S176M, S176A, S176T, S176F, T178L, T178V, and T178I (EU Numbering).
34. The antibody or fragment thereof of embodiment 33, wherein the HC1 CH1 consists of substitutions at F170, S183 and V185 (EU Numbering), and wherein the LC1 CL consists of a substitution at L135 (EU Numbering).
35. The antibody or fragment of embodiment 33, wherein the HC2 CH1 comprises or consists of SEQ ID NO: 9, and the LC2 CL comprises or consists of SEQ ID NO: 1 or 6.
36. The antibody or fragment thereof of embodiment 33, wherein the HC1 CH1 comprises a substitution at V185.
37. The antibody or fragment thereof of any one of embodiments 33-35, wherein the HC1 CH1 substitution at F170 (EU Numbering) is an F170I, or F170V substitution.
38. The antibody or fragment thereof of any one of embodiments 33-36, wherein the HC1 CH1 substitution at S183 (EU Numbering) is a S183L or S183I substitution.
39. The antibody or fragment thereof of any one of embodiments 33-37, wherein the HC1 CH1 substitution at V185 (EU Numbering) is a V185L substitution.
40. The antibody or fragment thereof of any one of embodiments 33-38, wherein the LC1 CL1 substitution at L135 (EU Numbering) is a L135F substitution.
41. The antibody or fragment thereof of embodiment 33, wherein the HC1 CH1 comprises or consists of an F170V, S1831 and V185L (EU Numbering) substitution, and the LC1 CL comprises or consists of a L135F (EU Numbering) substitution.
42. The antibody or fragment thereof of embodiment 33, wherein the HC1 CH1 comprises or consists of an F170I, S183L, and V185L (EU Numbering) substitution, and the LC1 CL comprises or consists of a L135F (EU Numbering) substitution.
43. An antibody or a fragment thereof comprising:
a first heavy chain (HC1), a second heavy chain (HC2), a first light chain (LC1),
and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG,
wherein the HC1 CH1 comprises or consists of a substitution at F126, F176, S189, V191, and C220 (EU Numbering), and the LC1 CL comprises or consists of a substitution at E124, L135, and C214 (lambda) or Q124, L135, and C214 (kappa) (EU Numbering),
wherein the HC1 CH1 does not include any substitution selected from L128F, A141M, A14IT, A141I, F170M, F170Y, F170S, F170A, S181I, S18IT, S181M, S183A, S183V, and V185A (EU Numbering), and wherein the LC1 CL does not include any substitution selected from F116A, F118V, S13IT, S131D, V133A, V133I, L135Y, L135V, S162A, S162M, T164S, S174A, S176M, S176A, S176T, S176F, T178L, T178V, and T178I (EU Numbering).
44. The antibody or fragment thereof of embodiments 43, wherein the HC CH1 substitution at F126 (EU Numbering) is an F126C substitution.
45. The antibody or fragment thereof of any one of embodiments 43-44, wherein the HC CH1 substitution at F170 (EU Numbering) is an F170V or F170I substitution.
46. The antibody or fragment thereof of any one of embodiments 43-45, wherein the HC CH1 substitution at S183 (EU Numbering) is a S183L, or S183I substitution.
47. The antibody or fragment thereof of any one of embodiments 43-46, wherein the HC CH1 substitution at V185 (EU Numbering) is a V185L substitution.
48. The antibody or fragment thereof of any one of embodiments 43-47, wherein the HC CH1 substitution at C220 (EU Numbering) is a C220S substitution.
49. The antibody or fragment thereof of any one of embodiments 43-48, wherein the LC CL substitution at position 124 (EU Numbering) is an E124C (lambda) or Q124C (kappa) substitution, optionally wherein the LC CL substitution at position 124 (EU Numbering) is an E124C (lambda) substitution, or optionally wherein the LC CL substitution at position 124 (EU Numbering) is a Q124C (kappa) substitution.
50. The antibody or fragment thereof of any one of embodiments 43-49, wherein the LC CL substitution at L135 (EU Numbering) is a L135F substitution.
51. The antibody or fragment thereof of any one of embodiments 43-50, wherein the LC CL substitution at C214 (EU Numbering) is a C214S substitution.
52. The antibody or fragment thereof of embodiment 43, wherein the HC CH1 comprises or consists of an F126C, F170V, S1831, V185L, and C220S (EU Numbering) substitution, and the LC CL comprises or consists of an E124C, L135F, and C214S (lambda) or Q124C, L135F, and C214S (kappa) (EU Numbering) substitution, optionally wherein the LC CL comprises or consists of an E124C, L135F, and C214S (lambda) (EU Numbering) substitution, or optionally wherein the LC CL comprises or consists of a Q124C, L135F, and C214S (kappa) (EU Numbering) substitution.
53. The antibody or fragment thereof of embodiment 43, wherein the HC CH1 comprises or consists of an F126C, F170I, S183L, V185L, and C220S (EU Numbering) substitution, and the LC CL comprises or consists of an E124C, L135F, and C214S (lambda) or Q124C, L135F, and C214S (kappa) (EU Numbering) substitution, optionally wherein the LC CL comprises or consists of an E124C, L135F, and C214S (lambda) (EU Numbering) substitution, or optionally wherein the LC CL comprises or consists of a Q124C, L135F, and C214S (kappa) (EU Numbering) substitution.
54. An antibody or a fragment thereof comprising:
   a first heavy chain (HC1), a second heavy chain (HC2), a first light chain (LC1),
   and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG,
   wherein the HC1 CH1 comprises or consists of a substitution at F126 and C220 (EU Numbering), the LC1 CL comprises or consists of a substitution at position 124 (E124 for lambda and Q124 for kappa), and C214 (EU Numbering), the HC2 CH1 comprises or consists of a substitution at F170, S183, and V185 (EU Numbering), and the LC2 CL comprises or consists of a substitution L135 (EU Numbering), wherein the HC1 CH1 and/or HC2 CH1 does not include any substitution selected from L128F, A141M, A141T, A141I, F170M, F170Y, F170S, F170A, S181I, S18IT, S181M, S183A, S183V, and V185A (EU Numbering),
   and wherein the LC1 CL and/or LC2 CL does not include any substitution selected from F116A, F118V, S13IT, S131D, V133A, V133I, L135Y, L13SV, S162A, S162M, T164S, S174A, S176M, S176A, S176T, S176F, T178L, T178V, and T178I (EU Numbering).
55. The antibody of embodiment 54, wherein the HC1 CH1 substitution at F126 (EU Numbering) is an F126C substitution, and the LC1 CL substitution at position 124 (EU Numbering) is an E124C substitution for lambda or Q124C for kappa LC, optionally wherein the LC1 CL substitution at position 124 (EU Numbering) is an E124C substitution for lambda, or optionally wherein the LC1 CL substitution at position 124 (EU Numbering) is a Q124C for kappa LC.
56. The antibody of any one of embodiments 54-55, wherein the HC CH1 substitution at C220 (EU Numbering) is a C220S substitution.
57. The antibody of any one of embodiments 54-56, wherein the LC CL substitution at C214 (EU Numbering) is a C214S substitution.
58. The antibody or fragment thereof of any one of embodiments 26-57, wherein the antibody comprises two heavy chain/light chain pairs, wherein the first pair is HC1/LC1, and the second pair is HC2/LC2.
59. The antibody of any one of embodiments 26-57, further comprising an HC1 and HC2 pair.
60. The antibody of embodiment 59, wherein the HC1 and HC2 are paired through charge-charge interactions.
61. The antibody of any one of the embodiments 26-60, wherein each HC1 and HC2 further comprises a heavy chain constant domain (CH3), and wherein the CH3 domains of HC1 and HC2 together comprise a knob-into-hole construct.
62. The antibody of embodiment 61, wherein the HC1 CH3 comprises a knob and the HC2 CH3 comprises a hole.
63. The antibody of embodiment 61, wherein the HC1 CH3 comprises a hole and the HC2 CH3 comprises a knob.
64. The antibody of any one of the embodiments 62-63, wherein the HC1 CH3 and/or HC2 CH3 comprises or consists of a T366, L368, and Y407 (EU Numbering) substitution.
65. The antibody of embodiment 64, wherein the HC1 CH3 and/or HC2 CH3 substitution at T366 (EU Numbering) is a T366W or T366S substitution.
66. The antibody of any one of the embodiments 64-65, wherein the HC1 CH3 and/or HC2 CH3 substitution at L368 (EU Numbering) is a L368A substitution.
67. The antibody of any one of the embodiments 64-66, wherein the HC1 CH3 and/or HC2 CH3 substitution at Y407 (EU Numbering) is a Y407V substitution.
68. The antibody of any one of the embodiments 64-67, wherein the HC1 CH3 comprises or consists of a T366W (EU Numbering) substitution and the HC2 CH3 comprises or consists of a T366S, L368A, and Y407V (EU Numbering) substitution.
69. The antibody of any one of the embodiments 64-68, wherein the HC1 CH3 comprises or consists of a T366S, L368A, and Y407V (EU Numbering) substitution and the HC2 CH3 comprises or consists of a T366W (EU Numbering) substitution.
70. The antibody of any one of embodiments 26-69, wherein the LC1 and/or LC2 is a kappa light chain.
71. The antibody of any one of embodiments 26-69, wherein the LC1 and/or LC2 is a lambda light chain.
72. The antibody of any one of embodiments 26-69, wherein the LC1 is a kappa light chain and the LC2 is a lambda light chain.
73. The antibody of any one of embodiments 26-69, wherein the LC1 is a lambda light chain and the LC2 is a kappa light chain.
74. The antibody or fragment thereof of any one of embodiments 26-73, wherein the HC2 CH1 comprises or consists of SEQ ID NO: 9, and the LC2 CL comprises or consists of SEQ ID NO: 1 or 6.
75. The antibody of any one of embodiments 26-74, wherein the light chain (LC) variable domain (VL) framework region (FR) and/or the heavy chain (HC) variable domain (VH) framework region (FR) comprises one or more substitutions.
76. The antibody of embodiment 75, wherein the VL FR comprises a substitution at Q38, and/or G100, and/or G101 (EU Numbering).
77. The antibody of embodiment 75, wherein the VH FR comprises a substitution at Q39, and/or G44 (EU Numbering).
78. The antibody of any one of embodiments 75-77, wherein the VH FR comprises a substitution at Q39, and/or G44 (EU Numbering), and the VL FR comprises a substitution at Q38, and/or G100, and/or G101 (EU Numbering).
79. The antibody of any one of embodiments 76-78, wherein the VL FR substitution at Q38 comprises or consists of a substitution selected from the group consisting of a Q38D and a Q38K substitution (EU Numbering).
80. The antibody of any one of embodiments 76-79, wherein the VL FR substitution at G100 comprises or consists of a substitution selected from the group consisting of a G100D, a G100K, and a G100C substitution (EU Numbering).
81. The antibody of any one of embodiments 76-80, wherein the VL FR substitution at G101 comprises or consists of a substitution selected from the group consisting of G101D and G101K substitution (EU Numbering).
82. The antibody of any one of embodiments 77-81, wherein the VH FR substitution at Q39 comprises or consists of a substitution selected from the group consisting of a Q39D and a Q39K substitution (EU Numbering).
83. The antibody of any one of embodiments 77-82, wherein the VH FR substitution at G44 comprises or consists of a substitution selected from the group consisting of a G44K, a G44D, and a G44C substitution (EU Numbering) 84. The antibody of any one of embodiments 75-83, wherein the substitution of VH FR comprises or consists of a Q39K substitution, and the substitution of VL FR comprises or consists of a Q38D substitution.
85. The antibody of any one of embodiments 75-83, wherein the substitution of VH FR comprises or consists of a Q39D substitution, and the substitution of VL FR comprises or consists of a Q38K substitution.
86. The antibody of any one of embodiments 75-85, wherein the substitution of VH FR comprises or consists of a G44K substitution, and the substitution of VL FR comprises or consists of a G100D substitution.
87. The antibody of any one of embodiments 75-85, wherein the substitution of VH FR comprises or consists of a G44D substitution, and the substitution of VL FR comprises or consists of a G100K substitution.
88 The antibody of any one of embodiments 75-85, wherein the substitution of VH FR comprises or consists of a G44C substitution, and the substitution of VL FR comprises or consists of a G100C substitution.
89. The antibody of any one of embodiments 75-85, wherein the substitution of VH FR comprises or consists of a G44K substitution, and the substitution of VL FR comprises or consists of a G101D substitution.
90. The antibody of any one of embodiments 75-85, wherein the substitution of VH FR comprises or consists of a G44D substitution, and the substitution of VL FR comprises or consists of a G101K substitution.
91. The antibody of any one of embodiments 75-90, wherein the substitution of HC1 VH FR comprises or consists of a Q39K substitution, the substitution of LC1 VL FR comprises or consists of a Q38D substitution, the substitution of HC2 VH FR comprises or consists of a Q39D substitution, and the substitution of LC2 VL FR comprises or consists of a Q38K substitution.
92. The antibody of any one of embodiments 75-90, wherein the substitution of HC1 VH FR comprises or consists of a Q39D substitution, the substitution of LC1 VL FR comprises or consists of a Q38K substitution, the substitution of HC2 VH FR comprises or consists of a Q39K substitution, and the substitution of LC2 VL FR comprises or consists of a Q38D substitution.
93. The antibody of any one of embodiments 75-90, wherein the substitution of HC1 VH FR comprises or consists of a G44K substitution, the substitution of LC1 VL FR comprises or consists of a G100D substitution, the substitution of HC2 VH FR comprises or consists of a G44D substitution, and the substitution of LC2 VL FR comprises or consists of a G100K substitution.
94. The antibody of any one of embodiments 75-90, wherein the substitution of HC1 VH FR comprises or consists of a G44D substitution, the substitution of LC1 VL FR comprises or consists of a G100K substitution, the substitution of HC2 VH FR comprises or consists of a G44K substitution, and the substitution of LC2 VL FR comprises or consists of a G100D substitution.
95. The antibody of any one of embodiments 75-90, wherein the substitution of HC1 VH FR comprises or consists of a G44C substitution and the substitution of LC1 VL FR comprises or consists of a G100C substitution.
96. The antibody of any one of embodiments 75-90, wherein the substitution of HC2 VH FR comprises or consists of a G44C substitution and the substitution of LC1 VL FR comprises or consists of a G100C substitution.
97. The antibody of any one of embodiments 75-90, wherein the substitution of HC1 VH FR comprises or consists of a G44K substitution, the substitution of LC1 VL FR comprises or consists of a G101D substitution, the substitution of HC2 VH FR comprises or consists of a G44D substitution, and the substitution of LC2 VL FR comprises or consists of a G101K substitution.
98. The antibody of any one of embodiments 75-90, wherein the substitution of HC1 VH FR comprises or consists of a G44D substitution, the substitution of LC1 VL FR comprises or consists of a G101K substitution, the substitution of HC2 VH FR comprises or consists of a G44K substitution, and the substitution of LC2 VL FR comprises or consists of a G101D substitution.
99. The antibody of any one of embodiments 75-98, wherein the HC1 or HC2 VH comprises or consists of any one of SEQ ID NO: 53-64.
100. The antibody of any one of embodiments 75-99, wherein the LC1 or LC2 VL comprises or consists of any one of SEQ ID NO: 65-80.
101. The antibody of any one of embodiments 75-100, wherein the heavy chain and light chain of the HC1/LC1 pair and the heavy chain and light chain of the HC2/LC2 pair each comprise or consist of the following sequence, respectively:
SEQ ID NOs: 30, 43, 35 and 52; or
SEQ ID NOs: 35, 52, 30 and 43.
102. The antibody of any one of embodiments 75-100, wherein the heavy chain and light chain of the HC1/LC1 pair and the heavy chain and light chain of the HC2/LC2 pair each comprise or consist of the following sequence, respectively:
SEQ ID NOs: 29, 44, 36 and 51; or
SEQ ID NOs: 36, 51, 29 and 44.
103. The antibody of any one of embodiments 75-100, wherein the heavy chain and light chain of the HC1/LC1 pair and the heavy chain and light chain of the HC2/LC2 pair each comprise or consist of the following sequence, respectively:
SEQ ID NOs: 28, 39, 33 and 48; or SEQ ID NOs: 33, 48, 28 and 39.
104. The antibody of any one of embodiments 75-100, wherein the heavy chain and light chain of the HC1/LC1 pair and the heavy chain and light chain of the HC2/LC2 pair each comprise or consist of the following sequence, respectively:
SEQ ID NOs: 27, 40, 34 and 47; or
SEQ ID NOs: 34, 47, 27 and 40.
105. The antibody of any one of embodiments 75-100, wherein the heavy chain and light chain of the HC1/LC1 pair and the heavy chain and light chain of the HC2/LC2 pair each comprise or consist of the following sequence, respectively:
SEQ ID NOs: 26, 38, 31 and 45; or
SEQ ID NOs: 31, 45, 26 and 38.
106. The antibody of any one of embodiments 75-100, wherein the heavy chain and light chain of the HC1/LC1 pair and the heavy chain and light chain of the HC2/LC2 pair each comprise or consist of the following sequence, respectively:
SEQ ID NOs: 25, 37, 32 and 46; or
SEQ ID NOs: 32, 46, 25 and 37.
107. The antibody of any one of embodiments 75-100, wherein the heavy chain and light chain of the HC1/LC1 pair and the heavy chain and light chain of the HC2/LC2 pair each comprise or consist of the following sequence, respectively:
SEQ ID NOs: 28, 41, 33 and 50; or
SEQ ID NOs: 33, 50, 28 and 41.
108. The antibody of any one of embodiments 75-100, wherein the heavy chain and light chain of the HC1/LC1 pair and the heavy chain and light chain of the HC2/LC2 pair each comprise or consist of the following sequence, respectively:
SEQ ID NOs: 27, 42, 34 and 49; or
SEQ ID NOs: 34, 49, 27 and 42.
109. The antibody of any one of embodiments 101-108, wherein the HC1 comprises or consists of SEQ ID NO: 30, the LC1 comprises or consists of SEQ ID NO: 43, the HC2 comprises or consists of SEQ ID NO: 35, and the LC2 comprises or consists of SEQ ID NO: 52.
110. The antibody of any one of embodiments 101-108, wherein the HC1 comprises or consists of SEQ ID NO: 29, the LC1 comprises or consists of SEQ ID NO: 44, the HC2 comprises or consists of SEQ ID NO: 36, and the LC2 comprises or consists of SEQ ID NO: 51.
111. The antibody of any one of embodiments 101-108, wherein the HC1 comprises or consists of SEQ ID NO: 28, the LC1 comprises or consists of SEQ ID NO: 39, the HC2 comprises or consists of SEQ ID NO: 33, and the LC2 comprises or consists of SEQ ID NO: 48.
112. The antibody of any one of embodiments 101-108, wherein the HC1 comprises or consists of SEQ ID NO: 27, the LC1 comprises or consists of SEQ ID NO: 40, the HC2 comprises or consists of SEQ ID NO: 34, and the LC2 comprises or consists of SEQ ID NO; 47.
113. The antibody of any one of embodiments 101-108, wherein the HC1 comprises or consists of SEQ ID NO: 26, the LC1 comprises or consists of SEQ ID NO: 38, the HC2 comprises or consists of SEQ ID NO: 31, and the LC2 comprises or consists of SEQ ID NO: 45.
114. The antibody of any one of embodiments 101-108, wherein the HC1 comprises or consists of SEQ ID NO: 25, the LC1 comprises or consists of SEQ ID NO: 37, the HC2 comprises or consists of SEQ ID NO: 32, and the LC2 comprises or consists of SEQ ID NO: 46.
115. The antibody of any one of embodiments 101-108, wherein the HC1 comprises or consists of SEQ ID NO: 28, the LC1 comprises or consists of SEQ ID NO: 41, the HC2 comprises or consists of SEQ ID NO: 33, and the LC2 comprises or consists of SEQ ID NO: 50.
116. The antibody of any one of embodiments 101-108, wherein the HC1 comprises or consists of SEQ ID NO: 27, the LC1 comprises or consists of SEQ ID NO: 42, the HC2 comprises or consists of SEQ ID NO: 34, and the LC2 comprises or consists of SEQ ID NO: 49.
117. The antibody of any one of embodiments 101-108, wherein the HC1 comprises or consists of SEQ ID NO: 35, the LC1 comprises or consists of SEQ ID NO: 52, the HC2 comprises or consists of SEQ ID NO: 30, and the LC2 comprises or consists of SEQ ID NO: 43
118. The antibody of any one of embodiments 101-108, wherein the HC1 comprises or consists of SEQ ID NO: 36, the LC1 comprises or consists of SEQ ID NO: 51, the HC2 comprises or consists of SEQ ID NO: 29, and the LC2 comprises or consists of SEQ ID NO: 44.
119. The antibody of any one of embodiments 101-108, wherein the HC1 comprises or consists of SEQ ID NO: 33, the LC1 comprises or consists of SEQ ID NO: 48, the HC2 comprises or consists of SEQ ID NO: 28, and the LC2 comprises or consists of SEQ ID NO: 39.
120. The antibody of any one of embodiments 101-108, wherein the HC1 comprises or consists of SEQ ID NO: 34, the LC1 comprises or consists of SEQ ID NO: 47, the HC2 comprises or consists of SEQ TD NO: 27, and the LC2 comprises or consists of SEQ ID NO: 40.
121. The antibody of any one of embodiments 101-108, wherein the HC1 comprises or consists of SEQ ID NO: 31, the LC1 comprises or consists of SEQ ID NO: 45, the HC2 comprises or consists of SEQ ID NO: 26, and the LC2 comprises or consists of SEQ ID NO: 38.
122. The antibody of any one of embodiments 101-108, wherein the HC1 comprises or consists of SEQ ID NO: 32, the LC1 comprises or consists of SEQ ID NO: 46, the HC2 comprises or consists of SEQ ID NO: 25, and the LC2 comprises or consists of SEQ ID NO: 37.
123. The antibody of any one of embodiments 101-108, wherein the HC1 comprises or consists of SEQ ID NO: 33, the LC1 comprises or consists of SEQ ID NO: 50, the HC2 comprises or consists of SEQ ID NO: 28, and the LC2 comprises or consists of SEQ ID NO: 41.
124. The antibody of any one of embodiments 101-108, wherein the HC1 comprises or consists of SEQ ID NO: 34, the LC1 comprises or consists of SEQ ID NO: 49, the HC2 comprises or consists of SEQ ID NO: 27, and the LC2 comprises or consists of SEQ ID NO: 42.
125. A composition comprising one or more polynucleotides encoding the Fab and/or the antibody of any one of embodiments 1-124.
126. The composition of embodiment 125, further comprising one or more polynucleotides encoding an IgG Fab or antibody.
127. An expression vector or construct for expression of the one or more polynucleotides of any one of embodiments 125-126.
128. The expression vector or construct of embodiment 127, wherein the expression vector is a plasmid or viral vector.
129. A cell, comprising the one or more polynucleotides, expression vector or construct of any one of embodiments 125-128.
130. A cell expressing one or more polynucleotides encoding the Fab and/or antibody or fragment thereof of any one of embodiments 1-124.
131. The cell of any one of embodiments 129-130, wherein the cell is a mammalian cell or bacterial cell.
132. The cell of any one of embodiments 129-131, wherein the cell is a HEK293 cell, Chinese hamster ovary (CHO) cell, or a NS0 cell.
133. A pharmaceutical composition comprising the Fab and/or antibody or fragment thereof of any one of embodiments 1-124.
134. A composition comprising the cell of any one of embodiments 129-132, optionally wherein the composition is a cell culture.
135. A composition comprising a cell supernatant and/or cell lysate of the cell of any one of embodiments 129-132.
136. A composition comprising antibodies comprising two heavy chain/light chain pairs selected from the pairs of HC1/LC1, HC2/LC2, HC1/LC2 and HC2/LC1 of any one of embodiments 1-124, wherein at least 50, 55, 60, 65, 70, 75, 80, 85, 90, 95, or 100% of the antibodies comprise the HC1/LC1 and HC2/LC2 pairs.
137. The composition of embodiment 136, wherein the composition comprises none or not more than 0.1, 0.5, 1, 2, 3, 4, 5, 6, 7, 8, 9, 10, 15, 20, 25, 30, 35, 40, 45, or 50% of an antibody comprising either the HC1/LC2 or the HC2/LC1.
138. The composition of any one of embodiments 133-137, wherein the composition is a cell culture supernatant and/or cell lysate, and wherein the composition comprises at least 50, 55, 60, 65, 70, 75, 80, 85, 90, 95, or 100% antibodies comprising the HC1/LC1 and HC2/LC2 pairs prior to any enrichment of the composition for the antibodies comprising the HC1/LC1 and HC2/LC2 pairs.
139. The composition of embodiment 138, wherein the composition comprises none or not more than 0.1, 0.5, 1, 2, 3, 4, 5, 6, 7, 8, 9, 10, 15, 20, 25, 30, 35, 40, 45, or 50% of an antibody or fragment thereof comprising either of the HC1/LC2 or the HC2/LC1 pairs prior to any enrichment of the composition for antibodies comprising the HC1/LC1 and HC2/LC2 pairs.
140. A method of producing an antibody or a fragment thereof, the method comprising:
providing a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG,
wherein the HC1 CH1 comprises a substitution at F170 and S183, and a substitution at V185 (EU Numbering), and the LC1 CL comprises a substitution at L135 (EU Numbering),
wherein the HC1 CH1 does not include any substitution selected from L128F, A141M, A14IT, A141I, F170M, F170Y, F170S, F170A, S181I, S181T, S181M, S183A, S183V, and V185A (EU Numbering), and
wherein the LC1 CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (EU Numbering).
141. The method of embodiment 140, wherein the HC1 CH1 consists of substitutions at F170 and S183 (EU Numbering), or at F170, S183 and V185 (EU Numbering), and wherein the LC1 CL consists of a substitution at L135 (EU Numbering).
142. The method of producing an antibody or fragment thereof of embodiment 141, wherein the HC1 CH1 further comprises a substitution at V185.
143. The method of producing an antibody or fragment thereof of any one of embodiments 140-142, wherein the HC1 CH1 substitution at F170 (EU Numbering) is an F170I, or F170V substitution.
144. The method of producing an antibody or fragment thereof of any one of embodiments 140-143, wherein the HC1 CH1 substitution at S183 (EU Numbering) is a S183L or S183I substitution.
145. The method of producing an antibody or fragment thereof of any one of embodiments 140-144, wherein the HC1 CH1 substitution at V185 (EU Numbering) is a V185L substitution.
146. The method of producing an antibody or fragment thereof of any one of embodiments 140-145, wherein the LC1 CL1 substitution at L135 (EU Numbering) is a L135F substitution.
147. The method of producing an antibody or fragment thereof of any one of embodiments 140-146, wherein the HC1 CH1 consists of an F1701 and S183V (EU Numbering) substitution, and the LC1 CL consists of a L135F (EU Numbering) substitution.
148. The method of producing an antibody or fragment thereof of any one of embodiments 140-147, wherein the HC1 CH1 consists of an F1701, S183L, and V185L (EU Numbering) substitution, and the LC1 CL consists of a L135F (EU Numbering) substitution.
149. A method of producing an antibody or a fragment thereof, the method comprising:
providing a first heavy chain (HC1), a second heavy chain (HC2), a first light chain (LC1), and second (LC2) light chain,
wherein each HC1 and HC2 comprises or a heavy chain variable domain (VH) and a first heavy chain constant domain (CH1) of a human IgG,
wherein each LC1 and LC2 comprises a light chain variable domain (VL) and a light chain constant domain (CL) of a human IgG,
wherein the HC1 CH1 comprises or consists of a substitution at F126, F170, S183, V185, and C220 (EU Numbering),
wherein the LC1 CL comprises or consists of a substitution at E124, L135, and C214 (lambda) or Q124, L135, and C214 (kappa) (EU Numbering), optionally wherein the LC1 CL comprises or consists of a substitution at E124, L135, and C214 (lambda) (EU Numbering), or optionally wherein the LC1 CL comprises or consists of a substitution at Q124, L135, and C214 (kappa) (EU Numbering),
wherein the HC1 CH1 does not include any substitution selected from L128F, A141M, A141T, A141I, F170M, F170Y, F170S, F170A, S181I, S181T, S181M, S183A, S183V, and V185A (EU Numbering), and
wherein the LC1 CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (EU Numbering).
150. The method of embodiment 149, wherein the HC1 CH1 substitution at F126 (EU Numbering) is an F126C substitution.
151. The method of any one of embodiments 149-150, wherein the HC1 CH1 substitution at F170 (EU Numbering) is an F170V or F170I substitution.
152. The method of any one of embodiments 149-151, wherein the HC1 CH1 substitution at S183 (EU Numbering) is a S183L, or S183I substitution.
153. The method of any one of embodiments 149-152, wherein the HC1 CH1 substitution at V185 (EU Numbering) is a V185L substitution.
154. The method of any one of embodiments 149-153, wherein the HC1 CH1 substitution at C220 (EU Numbering) is a C220S substitution.
155. The method of any one of embodiments 149-154, wherein the LC1 CL substitution at position 124 (EU Numbering) is an E124C for lambda or Q124C for kappa LC substitution, optionally wherein the LC1 CL substitution at position 124 (EU Numbering) is an E124C for lambda LC substitution, or optionally wherein the LC1 CL substitution at position 124 (EU Numbering) is a Q124C for kappa LC substitution.
156. The method of embodiments 149-155, wherein the LC1 CL substitution at L135 (EU Numbering) is a L135F substitution.
157. The method of embodiments 149-156, wherein the LC1 CL substitution at C214 (EU Numbering) is a C214S substitution.
158. The method of embodiment 149, wherein the HC1 CH1 comprises or consists of an F126C, F170V, S183I, V185L, and C220S (EU Numbering) substitution, and the LC1 CL comprises or consists of an E124C, L135F, and C214S (lambda) or Q124C, L135F, and C214S (kappa) (EU Numbering) substitution, optionally wherein the LC1 CL comprises or consists of an E124C, L135F, and C214S (lambda) (EU Numbering) substitution, or optionally wherein the LC1 CL comprises or consists of a Q124C, L135F, and C214S (kappa) (EU Numbering) substitution.
159. The method of embodiment 149, wherein the HC1 CH1 comprises or consists of an F126C, F170I, S183L, V185L, and C220S (EU Numbering) substitution, and the LC1 CL comprises or consists of an E124C, L135F, and C214S (lambda) or Q124C, L135F, and C214S (kappa) (EU Numbering) substitution, optionally wherein the LC1 CL comprises or consists of an E124C, L135F, and C214S (lambda) (EU Numbering) substitution, or optionally wherein the LC1 CL comprises or consists of a Q124C, L135F, and C214S (kappa) (EU Numbering) substitution.
160. A method of producing an antibody or a fragment thereof, the method comprising:
providing a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG,
wherein the HC1 CH1 comprises or consists of a substitution at F126 and C220 (EU Numbering), the LC1 CL comprises or consists of a substitution at E124 and C214 (lambda) or Q124 and C214 (kappa) (EU Numbering), (optionally at E124 and C214 (lambda), or optionally at Q124 and C214 (kappa)), the HC2 CH1 comprises or consists of a substitution at F170, S183, and V185 (EU Numbering), and the LC2 CL comprises or consists of a substitution L135 (EU Numbering),
wherein the HC1 CH1 and/or HC2 CH1 does not include any substitution selected from L128F, A141M, A141T, A141I, F170M, F170Y, F170S, F170A, S181I, S181T, S181M, S183A, S183V, and V185A (EU Numbering), and wherein the LC1 CL and/or LC2 CL does not include any substitution selected from F116A, F118V, S13IT, S131D, V133A, V133I, L13SY, L135V, S162A, S162M, T164S, S174A, S176M, S176A, S176T, S176F, T178L, T178V, and T178I (EU Numbering).
161. The method of embodiment 160, wherein the HC1 CH1 substitution at F126 (EU Numbering) is an F126C substitution, and the LC1 CL substitution at position 124 (EU Numbering) is an E124C (lambda) or Q124C (kappa) substitution, optionally wherein the LC1 CL substitution at position 124 (EU Numbering) is an E124C (lambda) substitution, or optionally wherein the LC1 CL substitution at position 124 (EU Numbering) is a Q124C (kappa) substitution.
162. The method of any one of embodiments 160-161, wherein the HC1 CH1 substitution at C220 (EU Numbering) is a C220S substitution.
163. The antibody of any one of embodiments 160-162, wherein the LC1 CL substitution at C214 (EU Numbering) is a C214S substitution.
164. The method of any one of embodiments 160-163, wherein the HC2 CH1 substitution at F170 (EU Numbering) is an F170V or F170I substitution.
165. The method of any one of embodiments 160-164, wherein the HC2 CH1 substitution at S183 (EU Numbering) is a S183L, or S183I substitution.
166. The method of any one of embodiments 160-165, wherein the HC2 CH1 substitution at V185 (EU Numbering) is a V185L substitution.
167. The method of any one of embodiments 160-166, wherein the LC2 CL substitution at L135 (EU Numbering) is a L135F substitution.
168. The method of embodiment 160, wherein the HC1 CH1 comprises or consists of an F126C and C220S (EU Numbering) substitution, the LC1 CL comprises or consists of an E124C and C214S (lambda) or Q124C and C214S (kappa) (EU Numbering) substitution (optionally wherein the LC1 CL comprises or consists of an E124C and C214S (lambda) (EU Numbering) substitution, or optionally wherein the LC1 CL comprises or consists of a Q124C and C214S (kappa) (EU Numbering) substitution), the HC2 CH1 comprises or consists of an F170I, S183L, V185L (EU Numbering), and the LC2 CL comprises or consists of a L135F (EU Numbering) substitution.
169. The method of embodiment 160, wherein the HC1 CH1 comprises or consists of an F126C and C220S (EU Numbering) substitution, the LC1 CL comprises or consists of an E124C and C214S (lambda) or Q124C and C214S (kappa) (EU Numbering) substitution (optionally wherein the LC1 CL comprises or consists of an E124C and C214S (lambda) (EU Numbering) substitution, or optionally wherein the LC1 CL comprises or consists of a Q124C and C214S (kappa) (EU Numbering) substitution), the HC2 CH1 comprises or consists of an F170V, S183I, V185L (EU Numbering), and the LC2 CL comprises or consists of a L135F (EU Numbering) substitution.
170. The method of any one of embodiments 140-169, wherein antibodies are produced comprising two heavy chain/light chain pairs, wherein the first pair is HC1/LC1, and the second pair is HC2/LC2.
171. The method of any one of embodiments 140-170, wherein the antibody further comprises a HC1/HC2 pair.
172. The method of embodiment 171, wherein the HC1 and HC2 are paired through charge-charge interactions.
173. The method of any one of embodiments 140-172, wherein each HC1 and HC2 further comprises a heavy chain constant domain (CH3), and wherein the HC1 and HC2 CH3 together comprise a knob-into-hole construct.
174. The method of embodiment 173, wherein the HC1 CH3 and/or HC2 CH3 comprises a knob and the HC2 CH3 comprises a hole.
175. The method of embodiment 173, wherein the HC1 CH3 comprises a hole and the HC2 CH3 comprises a knob.
176. The method of any one of embodiments 171-175, wherein the HC1 CH3 and/or HC2 CH3 comprises or consists of a T366, L368, and Y407 (EU Numbering) substitution.
177. The method of any one of embodiments 171-176, wherein the HC1 CH3 and/or HC2 CH3 substitution at T366 (EU Numbering) is a T366W or T366S substitution.
178. The method of any one of embodiments 171-177, wherein the HC1 CH3 and/or HC2 CH3 substitution at L368 (EU Numbering) is a L368A substitution.
179. The method of any one of embodiments 171-178, wherein the HC1 CH3 and/or HC2 CH3 substitution at Y407 (EU Numbering) is a Y407V substitution.
180. The method of any one of embodiments 171-179, wherein the HC1 CH3 comprises or consists of a T366W (EU Numbering) substitution and the HC2 CH3 comprises or consists of a T366S, L368A, and Y407V (EU Numbering) substitution.
181. The method of any one of embodiments 171-180, wherein the HC1 CH3 comprises or consists of a T366S, L368A, and Y407V (EU Numbering) substitution and the HC2 CH3 comprises or consists of a T366W (EU Numbering) substitution.
182. The method of any one of embodiments 140-181, wherein the LC1 and/or LC2 is a kappa light chain.
183. The method of any one of embodiments 140-181, wherein the LC1 and/or LC2 is a lambda light chain.
184. The method of any one of embodiments 140-181, wherein the LC1 is a lambda light chain and the LC2 is a kappa light chain.
185. The method of any one of embodiments 140-181, wherein the LC1 is a kappa light chain and the LC2 is a lambda light chain.
186. The method of any one of embodiments 140-185, wherein the VL FR comprises a substitution at Q38, and/or G100, and/or G101 (EU Numbering).
187. The method of any one of embodiments 140-186, wherein the VH FR comprises a substitution at Q39, and/or G44 (EU Numbering).
188. The method of any one of embodiments 140-187, wherein the VH FR comprises a substitution at Q39, and/or G44 (EU Numbering), and the VL FR comprises a substitution at Q38, and/or G100, and/or G101 (EU Numbering)
189. The method of any one of embodiments 140-188, wherein the VL FR substitution at Q38 comprises or consists of a substitution selected from the group consisting of a Q38D and a Q38K substitution (EU Numbering).
190. The method of any one of embodiments 140-188, wherein the VL FR substitution at G100 comprises or consists of a substitution selected from the group consisting of a G100D, a G100K, and a G100C substitution (EU Numbering).
191. The method of any one of embodiments 140-188, wherein the VL FR substitution at G101 comprises or consists of a substitution selected from the group consisting of a G10ID and G101K substitution (EU Numbering).
192. The method of any one of embodiments 140-188, wherein the VH FR substitution at Q39 comprises or consists of a substitution selected from the group comprising a Q39D and a Q39K substitution (EU Numbering).
193. The method of any one of embodiments 140-188, wherein the VH FR substitution at G44 comprises or consists of a substitution selected from the group consisting of a G44K, G44D, and G44C substitution (EU Numbering).
194. The method of any one of embodiments 140-193, wherein the substitution of the VH FR comprises or consists of a Q39K substitution, and the substitution of the VL FR comprises or consists of a Q38D substitution.
195. The method of any one of embodiments 140-193, wherein the substitution of the VH FR comprises or consists of a Q39D substitution, and the substitution of the VL FR comprises or consists of a Q38K substitution.
196. The method of any one of embodiments 140-193, wherein the substitution of the VH FR comprises or consists of a G44K substitution, and the substitution of the VL FR comprises or consists of a G100D substitution.
197. The method of any one of embodiments 140-193, wherein the substitution of the VH FR comprises or consists of a G44D substitution, and the substitution of the VL FR comprises or consists of a G100K substitution.
198. The method of any one of embodiments 140-193, wherein the substitution of the VH FR comprises or consists of a G44C substitution, and the substitution of the VL FR comprises or consists of a G100C substitution.
199. The method of any one of embodiments 140-193, wherein the substitution of the VH FR comprises or consists of a G44K substitution, and the substitution of the VL FR comprises or consists of a G101D substitution.
200. The method of any one of embodiments 140-193, wherein the substitution of the VH FR comprises or consists of a G44D substitution, and the substitution of the VL FR comprises or consists of a G101K substitution.
201. The method of any one of embodiments 140-193, wherein the substitution of the HC1 VH FR comprises or consists of a Q39K substitution, the substitution of the LC1 VL FR comprises or consists of a Q38D substitution, the substitution of the HC2 VH FR comprises or consists of a Q39D substitution, and the substitution of the LC2 VL FR comprises or consists of a Q38K substitution.
202. The method of any one of embodiments 140-193, wherein the substitution of the HC1 VH FR comprises or consists of a Q39D substitution, the substitution of the LC1 VL FR comprises or consists of a Q38K substitution, the substitution of the HC2 VH FR comprises or consists of a Q39K substitution, and the substitution of the LC2 VL FR comprises or consists of a Q38D substitution.
203. The method of any one of embodiments 140-193, wherein the substitution of the HC1 VH FR comprises or consists of a G44K substitution, the substitution of the LC1 VL FR comprises or consists of a G100D substitution, the substitution of the HC2 VH FR comprises or consists of a G44D substitution, and the substitution of the LC2 VL FR comprises or consists of a G100K substitution.
204. The method of any one of embodiments 140-193, wherein the substitution of the HC1 VH FR comprises or consists of a G44D substitution, the substitution of the LC1 VL FR comprises or consists of a G100K substitution, the substitution of the HC2 VH FR comprises or consists of a G44K substitution, and the substitution of the LC2 VL FR comprises or consists of a G100D substitution.
205. The method of any one of embodiments 140-193, wherein the substitution of the HC1 VH FR comprises or consists of a G44C substitution and the substitution of the LC1 VL FR comprises or consists of a G100C substitution.
206. The method of any one of embodiments 140-193, wherein the substitution of the HC2 VH FR comprises or consists of a G44C substitution and the substitution of the LC1 VL FR comprises or consists of a G100C substitution.
207. The method of any one of embodiments 140-193, wherein the substitution of the HC1 VH FR comprises or consists of a G44K substitution, the substitution of the LC1 VL FR comprises or consists of a G101D substitution, the substitution of the HC2 VH FR comprises or consists of a G44D substitution, and the substitution of the LC2 VL FR comprises or consists of a G101K substitution.
208. The method of any one of embodiments 140-193, wherein the substitution of the HC1 VH FR comprises or consists of a G44D substitution, the substitution of the LC1 VL FR comprises or consists of a G101K substitution, the substitution of the HC2 VH FR comprises or consists of a G44K substitution, and the substitution of the LC2 VL FR comprises or consists of a G101D substitution.
209. The method of any one of embodiments 140-208, wherein the HC1 or HC2 VH comprises or consists of any one of SEQ ID NO: 53-64.
210. The method of any one of embodiments 140-209, wherein the LC1 or LC2 VL comprises or consists of any one of SEQ ID NO: 65-80.
211. The method of any one of embodiments 140-210, wherein the heavy chain and light chain of the HC1/LC1 pair and the heavy chain and light chain of the HC2/LC2 pair each comprise or consist of the following sequence, respectively:
SEQ ID NOs: 30, 43, 35 and 52; or
SEQ ID NOs: 35, 52, 30 and 43.
212. The method of any one of embodiments 140-210, wherein wherein the heavy chain and light chain of the HC1/LC1 pair and the heavy chain and light chain of the HC2/LC2 pair each comprise or consist of the following sequence, respectively:
SEQ ID NOs: 29, 44, 36 and 51; or
SEQ ID NOs: 36, 51, 29 and 44.
213. The method of any one of embodiments 140-210, wherein wherein the heavy chain and light chain of the HC1/LC1 pair and the heavy chain and light chain of the HC2/LC2 pair each comprise or consist of the following sequence, respectively:
SEQ ID NOs: 28, 39, 33 and 48; or
SEQ ID NOs: 33, 48, 28 and 39.
214. The method of any one of embodiments 140-210, wherein the heavy chain and light chain of the HC1/LC1 pair and the heavy chain and light chain of the HC2/LC2 pair each comprise or consist of the following sequence, respectively:
SEQ ID NOs: 27, 40, 34 and 47; or
SEQ ID NOs: 34, 47, 27 and 40.
215. The method of any one of embodiments 140-210, wherein the heavy chain and light chain of the HC1/LC1 pair and the heavy chain and light chain of the HC2/LC2 pair each comprise or consist of the following sequence, respectively:
SEQ ID NOs: 26, 38, 31 and 45; or
SEQ ID NOs: 31, 45, 26 and 38.
216. The method of any one of embodiments 140-210, wherein the heavy chain and light chain of the HC1/LC1 pair and the heavy chain and light chain of the HC2/LC2 pair each comprise or consist of the following sequence, respectively:
SEQ ID NOs: 25, 37, 32 and 46; or
SEQ ID NOs: 32, 46, 25 and 37.
217. The method of any one of embodiments 140-210, wherein the heavy chain and light chain of the HC1/LC1 pair and the heavy chain and light chain of the HC2/LC2 pair each comprise or consist of the following sequence, respectively:
SEQ ID NOs: 28, 39, 33 and 50; or
SEQ ID NOs: 33, 50, 28 and 41.
218. The method of any one of embodiments 140-210, wherein wherein the heavy chain and light chain of the HC1/LC1 pair and the heavy chain and light chain of the HC2/LC2 pair each comprise or consist of the following sequence, respectively:
SEQ ID NOs: 27, 40, 34 and 49; or
SEQ ID NOs: 34, 49, 27 and 42.
219. The method of any one of embodiments 201-218, wherein the HC1 comprises or consists of SEQ ID NO: 30, the LC1 comprises or consists of SEQ ID NO: 43, the HC2 comprises or consists of SEQ ID NO: 35, and the LC2 comprises or consists of SEQ ID NO: 52.
220. The method of any one of embodiments 201-218, wherein the HC1 comprises or consists of SEQ ID NO: 29, the LC1 comprises or consists of SEQ ID NO: 44, the HC2 comprises or consists of SEQ ID NO: 36, and the LC2 comprises or consists of SEQ ID NO: 51.
221. The method of any one of embodiments 201-218, wherein the HC1 comprises or consists of SEQ ID NO: 28, the LC1 comprises or consists of SEQ ID NO: 39, the HC2 comprises or consists of SEQ ID NO: 33, and the LC2 comprises or consists of SEQ ID NO: 48.
222. The method of any one of embodiments 201-218, wherein the HC1 comprises or consists of SEQ ID NO: 27, the LC1 comprises or consists of SEQ ID NO: 40, the HC2 comprises or consists of SEQ ID NO: 34, and the LC2 comprises or consists of SEQ ID NO: 47.
223. The method of any one of embodiments 201-218, wherein the HC1 comprises or consists of SEQ ID NO: 26, the LC1 comprises or consists of SEQ ID NO: 38, the HC2 comprises or consists of SEQ ID NO. 31, and the LC2 comprises or consists of SEQ ID NO: 45.
224. The method of any one of embodiments 201-218, wherein the HC1 comprises or consists of SEQ ID NO: 25, the LC1 comprises or consists of SEQ ID NO: 37, the HC2 comprises or consists of SEQ ID NO: 32, and the LC2 comprises or consists of SEQ ID NO: 46.
225. The method of any one of embodiments 201-218, wherein the HC1 comprises or consists of SEQ ID NO: 28, the LC1 comprises or consists of SEQ ID NO: 41, the HC2 comprises or consists of SEQ ID NO: 33, and the LC2 comprises or consists of SEQ ID NO: 50.
226. The method of any one of embodiments 201-218, wherein the HC1 comprises or consists of SEQ ID NO: 27, the LC1 comprises or consists of SEQ ID NO: 42, the HC2 comprises or consists of SEQ ID NO: 34, and the LC2 comprises or consists of SEQ ID NO: 49.
227. The method of any one of embodiments 201-218, wherein the HC1 comprises or consists of SEQ ID NO: 35, the LC1 comprises or consists of SEQ ID NO: 52, the HC2 comprises or consists of SEQ ID NO: 30, and the LC2 comprises or consists of SEQ ID NO: 43.
228. The method of any one of embodiments 201-218, wherein the HC1 comprises or consists of SEQ ID NO: 36, the LC1 comprises or consists of SEQ ID NO: 51, the HC2 comprises or consists of SEQ ID NO: 29, and the LC2 comprises or consists of SEQ ID NO: 44.
229. The method of any one of embodiments 201-218, wherein the HC1 comprises or consists of SEQ ID NO: 33, the LC1 comprises or consists of SEQ ID NO: 48, the HC2 comprises or consists of SEQ ID NO: 28, and the LC2 comprises or consists of SEQ ID NO: 39 230. The method of any one of embodiments 201-218, wherein the HC1 comprises or consists of SEQ ID NO: 34, the LC1 comprises or consists of SEQ ID NO: 47, the HC2 comprises or consists of SEQ ID NO: 27, and the LC2 comprises or consists of SEQ ID NO: 40.
231. The method of any one of embodiments 201-218, wherein the HC1 comprises or consists of SEQ ID NO: 31, the LC1 comprises or consists of SEQ ID NO: 45, the HC2 comprises or consists of SEQ ID NO: 26, and the LC2 comprises or consists of SEQ ID NO: 38.
232. The method of any one of embodiments 201-218, wherein the HC1 comprises or consists of SEQ ID NO: 32, the LC1 comprises or consists of SEQ ID NO: 46, the HC2 comprises or consists of SEQ ID NO: 25, and the LC2 comprises or consists of SEQ ID NO: 37.
233. The method of any one of embodiments 201-218, wherein the HC1 comprises or consists of SEQ ID NO: 33, the LC1 comprises or consists of SEQ ID NO: 50, the HC2 comprises or consists of SEQ ID NO: 28, and the LC2 comprises or consists of SEQ ID NO: 41.
234. The method of any one of embodiments 201-218, wherein the HC1 comprises or consists of SEQ ID NO: 34, the LC1 comprises or consists of SEQ ID NO: 49, the HC2 comprises or consists of SEQ ID NO: 27, and the LC2 comprises or consists of SEQ ID NO: 42.
235. The method of any one of embodiments 140-234, wherein a plurality of antibodies comprising two heavy chain/light chain pairs are produced, and at least 50, 55, 60, 65, 70, 75, 80, 85, 90, 95, or 100% of the antibodies comprising two heavy chain/light chain pairs that are produced comprise HC1/LC1 and HC2/LC2.
236. The method of any one of embodiments 140-235, wherein none or not more than 0.1, 0.5, 1, 2, 3, 4, 5, 6, 7, 8, 9, 10, 15, 20, 25, 30, 35, 40, 45, or 50% of the antibodies comprising two heavy chain/light chain pairs that are produced comprise either a HC1/LC2 or a HC2/LC1.
237. The method of embodiment 236, wherein the percentage (%) of the antibodies comprising HC1/LC1 and HC2/LC2 pairs refers to the percentage of HC1/LC1 and HC2/LC2 pairs prior to any enrichment for antibodies comprising the HC1/LC1 and HC2/LC2 pairs.
238. The method of embodiment 236, wherein the percentage (%) of the antibodies comprising HC1/LC2 and HC2/LC1 pairs refers to the percentage of HC1/LC2 and HC2/LC1 pairs prior to any enrichment for antibodies comprising the HC1/LC1 and HC2/LC2 pairs.
239. The method of any one of embodiments 140-238, wherein the providing comprises providing one or more polynucleotides encoding the HC1, HC2, LC1, and LC2.
240. The method of embodiment 239, wherein the one or more polynucleotides encodes the LC1 and LC2 at a ratio that is, or is about, 8:1 to 1:8, optionally 1:1, 2:1, 3:1, 4:1, 5:1, 6:1, 7:1, 8:1, 9:1, or 10:1, optionally wherein the ratio is, or is about, 1:5
241. The method of embodiment 240, wherein the one or more polynucleotides encodes the HC1 and HC2 at a ratio that is, or is about, 8:1, 7:1, 6:1, 5:1, 4:1, 3:1, 2:1, 1:1, 1:2, 1:3, 1:4, 1:5, 1:6, 1:7, or 1:8, optionally 1:1, 1.5:1, or 2:1.
242. The method of embodiment 241, wherein the one or more polynucleotides encodes the HC1 and LC2 at a ratio that is, or is about, 1:0.5 to 1:3, optionally 1:0.5, 1:1, 1:1.5, 1:2, 1:2.5, or 1:3.
243. The method of embodiment 239, wherein the one or more polynucleotides encodes the HC1, HC2, LC1, and LC2 at a ratio that is, or is about, 1:1:1:1, 1:1:1.5:1.5, 1:1:2:1, or 2:2:5:1.
244. The method of any one of embodiments 239-243, wherein the one or more polynucleotides encodes an IgG Fab or antibody.
245. The method of any one of embodiments 141-239, wherein the providing comprises providing one or more expression vectors or constructs encoding the HC1, HC2, LC1, and LC2.
246. The method of embodiment 245, wherein the one or more expression vectors or constructs encodes the LC1 and LC2 at a ratio that is, or is about, 8:1 to 1:8, optionally, 1:1, 2:1, 3:1, 4:1, 5:1, 6:1, 7:1, 8:1, 9:1, or 10:1, optionally wherein the ratio is, or is about, 1:5.
247. The method of embodiment 246, wherein the one or more expression vectors or constructs encodes the HC1 and HC2 at a ratio that is, or is about, 8:1, 7:1, 6:1, 5:1, 4:1, 3:1, 2:1, 1:1, 1:2, 1:3, 1:4, 1:5, 1:6, 1:7, or 1:8, optionally 1:1, 1.5:1, or 2:1.
248. The method of embodiment 247, wherein the one or more expression vectors or constructs encodes the HC1 and LC2 at a ratio that is, or is about, 1:0.5 to 1:3, optionally 1:0.5, 1:1, 1:1.5, 1:2, 1:2.5, or 1:3.
249. The method of embodiment 245, wherein the one or more expression vectors or constructs encodes the HC1, HC2, LC1, and LC2 at a ratio that is, or is about, 1:1:1:1, 1:1:1.5:1.5, 1:1:2:1, or 2:2:5:1.
250. The method of any one of embodiments 245-249, wherein the one or more expression vectors is a plasmid(s) or viral vector(s).
251. The method of any one of embodiments 245-250, wherein the one or more expression vectors or constructs encode an IgG Fab or antibody.
252. The method of any one of embodiments 140-251, wherein the providing comprises providing a cell expressing the HC1, HC2, LC1, and LC2.
253. The method of embodiment 252, wherein the cell expresses the LC1 and LC2 at a ratio that is, or is about, 8:1 to 1:8, optionally 1:1, 2:1, 3:1, 4:1, 5:1, 6:1, 7:1, 8:1, 9:1, or 10:1, optionally wherein the ratio is, or is about, 1:5.
254. The method of embodiment 253, wherein the cell expresses the HC1 and HC2 at a ratio that is, or is about, 8:1, 7:1, 6:1, 5:1, 4:1, 3:1, 2:1, 1:1, 1:2, 1:3, 1:4, 1:5, 1:6, 1:7, or 1:8, optionally 1:1, 1.5:1, or 2:1.
255. The method of embodiment 254, wherein the cell expresses the HC1 and LC2 at a ratio that is, or is about, 1:0.5 to 1:3, optionally 1:0.5, 1:1, 1:1.5, 1:2, 1:2.5, or 1:3.
256. The method of embodiment 252, wherein the cell expresses the HC1, HC2, LC1, and LC2 at a ratio that is, or is about, 1:1:1:1, 1:1:1.5:1.5, 1:1:2:1, or 2:2:5:1.
257. The method of any one of embodiments 252-256, wherein the cell expresses an IgG Fab or antibody.
258. The method of any one of embodiments 252-257, wherein the cell is a mammalian cell or bacterial cell.
259. The method of any one of embodiments 252-257, wherein the cell is a HEK293 cell, Chinese hamster ovary (CHO) cell, or a NS0 cell.
260. The method of any one of embodiments 140-259, further comprising extraction and/or purification of the antibody or fragment thereof.
261. The method of embodiment 260, wherein the antibody or fragment thereof is extracted and/or purified from a cell supernatant or cell lysate.
262. The Fab of any one of embodiments 1-30, wherein the Fab is a chimeric, humanized, or human Fab.
263. The antibody, composition, cell, expression vector or construct, polynucleotide, or method of any one of embodiments 31-261, wherein the antibody comprises or consists of an IgG antibody.
264. The antibody, composition, cell, expression vector or construct, polynucleotide, or method of embodiment 263, wherein the IgG antibody is IgG1, IgG2, IgG3, or IgG4.
265. The antibody, composition, cell, expression vector or construct, polynucleotide, or method of any one of embodiments 263-264, wherein the antibody is a chimeric, humanized, or human antibody.
266. Any one of the preceding embodiments, wherein the fragment comprises and antigen binding fragment.

### BRIEF DESCRIPTION OF THE DRAWINGS

FIG. 1 is an illustration of an embodiment of an antigen-binding fragment (Fab).

FIG. 2 is an illustration of an embodiment of an antibody.

FIG. 3 is a flow chart of an embodiment of a method for screening compensating mutations in CL and CH1 domains that convey LC and HC specific pairing for an antibody or a fragment thereof.

FIG. 4 is an illustrative representation of an embodiment of using light chain with L135F to interfere with the correct pairing of WT heavy chains to a WT light chain.

FIG. 5 is an illustrative representation of an embodiment of using wt light chain to interfere with the correct pairing of mutated heavy chains to a mutated light chain.

FIG. 6A is a table indicating the identity of an embodiment of samples in each lane of the SDS-page gel in FIG. 6B using the screening strategies illustrated in FIGS. 4 & 5.

FIG. 6B is an SDS-PAGE gel of an embodiment of different HC/LC pairings under non-reducing conditions. Mutation sets that enhance LC/HC pairing specificity will enhance percentage of the correctly assembled intact antibodies (upper band of two LCs and two HCs) in the presence of similar amount of competing unchanged wt LC-JT7 or 11/paired with LC-F achieved comparable wt IgG's correct assembly in the presence of competing wt LC.

FIG. 7A is a table indicating the identity of an embodiment of samples in each lane of the SDS-page gel in FIG. 7B using the screening strategies illustrated in FIGS. 4 & 5.

FIG. 7B is an SDS-page gel of an embodiment of different HC/LC pairings under reducing conditions.

FIG. 8 is a series of chromatograms of an embodiment of quantification by RP-HPLC of correct LC-HC pairing in positive controls and selected mutation sets.

FIG. 9A is an illustrative representation of an embodiment of correct pairing of heavy chains of JT7 and JT11 dimerizing with the light chain with L135F mutation in an example of bispecific antibody (bsMab).

FIG. 9B is a chromatogram of an embodiment of purity and correct assembly analyses by RP-HPLC for purified bispecific antibodies using LC/HC pairing strategies of alternative disulfide (R2), JT7/F and JT11/F Dimerization pairs.

FIG. 9C is an SDS-page gel of an embodiment of correctly assembled bispecific antibodies (LC-HC dimers) under reducing and non-reducing conditions.

FIG. 10A is a graph of an embodiment of the neutralizing activity of bispecific antibody's (bsMab) Fab arm 1 in a cytokine A blockage assay by bispecific antibodies in HEK-293 reporter cells.

FIG. 10B is a graph of an embodiment of the neutralizing activity of bispecific antibody's (bsMab) Fab arm 2 in a cytokine B blockage assay by bispecific antibodies in HEK-293 reporter cells.

FIG. 11A is an SDS-page gel of an embodiment of bsMabs of R2, R2 plus JT7/F and R2 plus JT11/F expressed in 293 cells under reducing and non-reducing conditions.

FIG. 11B is a table of quantification of an embodiment of the yield for expression by ProA capture and percentage of correct assembly in the ProA pool analyzed by RP-HPLC in FIG. 11C.

FIG. 11C is a set of chromatograms of an embodiment of quantification by RP-HPLC for correct LC-HC pairings in the Protein A pools of bsMabs of R2, R2 plus JT7/F and R2 plus JT11/F which are expressed in 293 cells.

FIG. 12 is a table of an embodiment of CL sequences in wild type and mutant CL domains of lambda and kappa LC.

FIG. 13 is a table of an embodiment of CH1 sequences in wild type and mutant CH1 domains of IGHG1.

FIG. 14 is a table showing some embodiments of sequence alignments of wild type and mutant CH1 domains of IGHG1. Blank residues indicate consensus to IGHG1 CH1. Substituted residues are indicated at each position as shown.

FIG. 15 is a table showing some embodiments of sequence alignments of lambda and mutant CL domains. Blank residues indicate consensus to lambda CL. Substituted residues are indicated at each position as shown.

FIG. 16 is a table showing some embodiments of sequence alignments of kappa and mutant CL domains. Blank residues indicate consensus to Lambda C L. Substituted residues are indicated at each position as shown.

FIG. 17A is a bar graph depicting an embodiment of quantification of bispecific antibody expression in CHO cells.

FIG. 17B is a bar graph depicting an embodiment of quantification of ProA pools analyzed by capillary electrophoresis sodium dodecyl sulfate (CE-SDS).

FIG. 17C is a bar graph depicting an embodiment of quantification of mismatched species by Mass Spec.

FIG. 18A is a chromatogram depicting an embodiment of quantification of Fab fragment parings as analyzed by LC-MS.

FIG. 18B is a chromatogram depicting an embodiment of quantification of Fab fragment pairings following polishing as analyzed by LC-MS.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless specified otherwise, all technical and scientific terms used herein have the same meaning as is commonly understood by one of skill in the art to which the claimed subject matter belongs when read in light of the specification. It is to be understood that the foregoing general description and the following detailed description are exemplary and explanatory only and are not restrictive of any subject matter claimed.

The section headings used herein are for organizational purposes only and are not to be construed as limiting the subject matter described.

Several approaches have been established to produce antibodies with two different antigen binding fragment (Fab) arms, for example WO2021155461. However, promiscuous pairing of antibody heavy chain (HC) and light chains (LC) remains a challenge for their efficient production. Accordingly, some embodiments herein are directed to Fabs, antibodies, and fragments thereof, having improved heavy HC-HC and HC-LC pairing efficiency. Also disclosed herein are methods of producing Fabs, antibodies, and fragments thereof, having improved heavy HC-HC and HC-LC pairing efficiency.

For example, some embodiments herein relate to an antigen-binding fragment (Fab) comprising: a heavy chain variable domain (VH) and a heavy chain constant domain (CH1), a light chain variable domain (VL), and a light chain constant domain (CL), wherein the HC CH1 consists of a substitution at F170, S183 and V185 and the LC1 CL consists of a substitution at L135 (EU Numbering).

Some embodiments herein are directed to an antibody or a fragment thereof comprising: a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1), and each LC1 and LC2 comprises a light chain variable domain (VL) and a light chain constant domain (CL), wherein the HC1 CH1 consists of a substitution at F170, S183 and V185 and the LC1 CL consists of a substitution at L135 (EU Numbering). In some embodiments, the fragment comprises an antigen binding fragment.

Some embodiments herein are directed to a method of producing an antibody or a fragment thereof, the method comprising: providing a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1), and each LC1 and LC2 comprises a light chain variable domain (VL) and a light chain constant domain (CL), wherein the HC1 CH1 consists of a substitution at F170, S183 and V185, and the LC1 CL consists of a substitution at L135 (EU Numbering). In some embodiments, the fragment comprises an antigen binding fragment.

### Terminology

The articles “a” and “an” have its plain and ordinary meaning as read in light of the specification and refer to one or to more than one (for example, at least one) of the grammatical object of the article. By way of example, “an element” means one element or more than one element.

Throughout this specification, unless the context requires otherwise, the words “comprise,” “comprises,” and “comprising” has their plain and ordinary meaning as read in light of the specification and will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “polypeptide”, “peptide”, and “protein” are used interchangeably herein and have their plain and ordinary meaning as read in light of the specification, including reference to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component.

As used herein the term “amino acid” has its plain and ordinary meaning as read in light of the specification and refers to either natural and/or unnatural or synthetic amino acids, including glycine and both the D or L optical isomers, and amino acid analogs and peptidomimetics.

A polypeptide or amino acid sequence “derived from” a designated protein has its plain and ordinary meaning as read in light of the specification and refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence. Peptide sequences having at least 80%, 85%, 90%, 95%, 99%, or 100% homology to any one of the peptide sequences disclosed herein and having the same or similar functional properties are envisioned. The percent homology may be determined according to amino acid substitutions, deletions, or additions between two peptide sequences. Peptide sequences having some percent homology to any one of the peptide sequences disclosed herein may be produced and tested by one skilled in the art through conventional methods. The % homology or % identity of two sequences is well understood in the art and can be calculated by the number of conserved amino acids or nucleotides relative to the length of the sequences.

As used herein, the term “antibody” or “immunoglobulin” has its plain and ordinary meaning as read in light of the specification and denotes the meaning ascribed to it by one of skill in the art, and further it is intended to include any polypeptide chain-containing molecular structure with a specific shape that fits to and recognizes an epitope, where one or more non-covalent binding interactions stabilize the complex between the molecular structure and the epitope. Antibodies utilized in the disclosed embodiments may be polyclonal antibodies, although monoclonal antibodies are preferred because they may be reproduced by cell culture or recombinantly and can be modified to reduce their antigenicity.

Naturally occurring immunoglobulin are tetrameric glycoproteins, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. The amino terminal ends of the polypeptide chains show considerable variation in amino acid composition and are referred to as the variable heavy (VH) and variable light (VL) regions to distinguish them from the relatively constant heavy (CH) and constant light (CL) regions. Each LC consists of one VL and one CL. The HCs consist of a VH, and three constant heavy domains CH1, CH2, and CH3. Antibodies can be separated functionally into VL and VH domains that bind to antigens and CL and CH domains that specify effector functions such as activation of complement or binding to Fe receptors. Each variable domain can be split into three regions of sequence variability, termed the complementarity determining regions, or CDRs, and four regions of relatively constant sequence termed the framework regions, or FRs. The three CDRs of the HC are paired with the three CDRs of the LC to form the antigen binding site, as classically defined. There are five main classes of HC CH domains. Each class defines the IgM, IgG, IgA, IgD, and IgE isotypes. IgG can be subdivided into four subclasses, IgG1, IgG2, IgG3, and IgG4, each with its own biologic properties; and IgA can similarly be split into IgAQ1 and IgA2. The constant domains of the HC can be switched to allow altered effector function while maintaining antigen specificity.

As used herein, the term “chimeric antibody” has its plain and ordinary meaning as read in light of the specification and refers to an antibody or a fragment thereof comprising the variable domain of an antibody from one species and the constant domain of an antibody from a different species. A “humanized antibody” has its plain and ordinary meaning as read in light of the specification and refers to an antibody or a fragment thereof comprising the variable or constant domain of a human antibody and the variable or constant domain of an antibody from a different species. In some embodiments, the fragment comprises an antigen binding fragment.

In addition to entire immunoglobulins (or their recombinant counterparts), immunoglobulin fragments or “binding fragments” comprising the epitope binding site (e.g., Fab′, F(ab′)2, single-chain variable fragment (scFv), diabody, minibody, nanobody, singledomain antibody (sdAb), or other fragments) are useful as antibody moieties in the disclosed embodiments. Such antibody fragments may be generated from whole immunoglobulins by ricin, pepsin, papain, or other protease cleavage. Minimal immunoglobulins may be designed utilizing recombinant immunoglobulin techniques. For instance, “Fv” immunoglobulins for use in the disclosed embodiments may be produced by linking a variable light chain region to a variable heavy chain region via a peptide linker (e.g., poly-glycine or another sequence which does not form an alpha helix or beta sheet motif). Nanobodies or single-domain antibodies can also be derived from alternative organisms, such as dromedaries, camels, llamas, alpacas, or sharks. In some embodiments, antibodies can be conjugates, e.g. pegylated antibodies, drug, radioisotope, or toxin conjugates. Monoclonal antibodies directed against a specific epitope, or combination of epitopes, will allow for the targeting and/or depletion of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (e.g. U.S. Pat. No. 5,985,660, hereby expressly incorporated by reference in its entirety).

As used herein the term “Fc region” has its plain and ordinary meaning as read in light of the specification and refers to a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

In some embodiments, definitive delineation of a CDR and identification of residues comprising the binding site of an antibody is accomplished by solving the structure of the antibody and/or solving the structure of the antibody-ligand complex. In certain embodiments, which can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the IMGT approach (Lefranc et al., 2003, Dev Comp Immunol. 27:55-77), computational programs such as Paratome (Kunik et al., 2012, Nucl Acids Res. W521-4), the AbM definition, and the conformational definition.

The Kabat definition is a standard for numbering the residues in an antibody and is typically used to identify CDR and other antibody regions. See, e.g., Johnson & Wu, 2000, Nucleic Acids Res., 28:214-8. The Chothia definition is similar to the Kabat definition, but the Chothia definition takes into account positions of certain structural loop regions. See, e.g., Chothia et al., 1986, J. Mol. Biol., 196:901-17; Chothia et al., 1989, Nature, 342:877-83. The AbM definition uses an integrated suite of computer programs produced by Oxford Molecular Group that model antibody structure. See, e.g., Martin et al., 1989, Proc Natl Acad Sci (USA), 86:9268-9272; “AbM.TM., A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:73245. As used herein, a CDR, Fab Fragment, CL, CH1, CH2, CH3, VL, and/or VH have their plain and ordinary meaning as read in light of the specification and may refer to a CDR, Fab Fragment, CL, CH1, CH2, CH3, VL, and/or VH defined by any approach known in the art, including combinations of approaches. The methods used herein may utilize a CDR, CL, CH1, CH2, CH3, VL, and/or VH defined according to any of these approaches. For any given embodiment containing more than one CDR, Fab, CL, CH1, CH2, CH3, VL, and/or VH, the CDRs, Fabs, CLs, CH1s, CH2s, CH3s, VLs, and/or VHs may be defined in accordance with any of Kabat, Chothia, extended, IMGT, Paratome, AbM, and/or conformational definitions, or a combination of any of the foregoing.

As used herein, the term “constant region” as it relates to an antibody has its plain and ordinary meaning as read in light of the specification and refers to the constant region of the antibody light chain or the constant region of the antibody heavy chain, either alone or in combination.

A “variable region” of an antibody has its plain and ordinary meaning as read in light of the specification and refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196 (4): 901-917, 1987).

As used herein, the term “antigen binding molecule” has its plain and ordinary meaning as read in light of the specification and refers to a molecule that comprises an antigen binding portion that binds to an antigen and, optionally, a scaffold or framework portion that allows the antigen binding portion to adopt a conformation that promotes binding of the antigen binding portion or provides some additional properties to the antigen binding molecule. In some embodiments, the antigen binding portion comprises at least one CDR from an antibody that binds to the antigen. In some embodiments, the antigen binding portion comprises all three CDRs from a heavy chain of an antibody that binds to the antigen or from a light chain of an antibody that binds to the antigen. In some embodiments, the antigen binding portion comprises all six CDRs from an antibody that binds to the antigen (three from the heavy chain and three from the light chain). In some embodiments, the antigen binding portion is an antibody fragment.

Non-limiting examples of antigen binding molecules include antibodies, antibody fragments (e.g., an antigen binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)2 fragment, an Fv fragment, an Fd fragment, and a complementarity determining region (CDR) fragment. These molecules can be derived from any mammalian source, such as human, mouse, rat, rabbit, pig, dog, cat, horse, donkey, guinea pig, goat, or camelid. Antibody fragments may compete for binding of a target antigen with an intact antibody and the fragments may be produced by the modification of intact antibodies (e.g. enzymatic or chemical cleavage) or synthesized de novo using recombinant DNA technologies or peptide synthesis. The antigen binding molecule can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding molecule as well as wholly synthetic scaffolds comprising, for example, a biocompatible polymer. See, for example, Korndorfer et al., 2003, Proteins: Structure, Function, and Bioinformatics, Volume 53, Issue 1:121-129 (2003); Roque et al., Biotechnol. Prog. 20:639654 (2004). In addition, peptide antibody mimetics (“PAMs”) can be used, as well as scaffolds based on antibody mimetics utilizing fibronectin components as a scaffold.

An antigen binding molecule can also include a protein comprising one or more antibody fragments incorporated into a single polypeptide chain or into multiple polypeptide chains. For instance, antigen binding molecule can include, but are not limited to, a diabody (see, e.g., EP 404,097; WO 93/11161; and Hollinger et al., Proc. Natl. Acad. Sci. USA, Vol. 90:6444-6448, 1993); an intrabody; a domain antibody (single VL or VH domain or two or more VH domains joined by a peptide linker; see Ward et al., Nature, Vol. 341:544546, 1989); a maxibody (2 scFvs fused to Fc region, see Fredericks et al., Protein Engineering, Design & Selection, Vol. 17:95-106, 2004 and Powers et al., Journal of Immunological Methods, Vol. 251:123-135, 2001); a triabody; a tetrabody; a minibody (scFv fused to CH3 domain; see Olafsen et al., Protein Eng Des Sel., Vol. 17:315-23, 2004); a peptibody (one or more peptides attached to an Fc region, see WO 00/24782); a linear antibody (a pair of tandem Fd segments (VH—CH1-VH-CH1) which, together with complementary light chain polypeptides, form a pair of antigen binding regions, see Zapata et al., Protein Eng., Vol. 8:1057-1062, 1995); a small modular immunopharmaceutical (see U.S. Patent Publication No. 20030133939); and immunoglobulin fusion proteins (e.g. IgG-scFv, IgG-Fab, 2scFv-IgG, 4scFv-IgG, VH-IgG, IgG-VH, and Fab-scFv-Fc). In certain embodiments, an antigen binding molecule can have, for example, the structure of an immunoglobulin.

As used herein, the term “antigen binding fragment” (Fab) has its plain and ordinary meaning as read in light of the specification and refers to the portion of an antibody that binds an antigen. As used herein, Fab may refer to both monovalent F(ab) and divalent F(ab′)2 fragments. An antibody digested by the enzyme papain yields two F(ab) fragments of about 50 kDa each and an Fc fragment. In contrast, F(ab′)2 fragment antibodies may be generated by pepsin digestion of whole IgG antibodies to remove most of the Fc region while leaving intact some of the hinge region. F(ab′)2 fragments have two antigen-binding F(ab) portions linked together by disulfide bonds, and therefore are divalent with a molecular weight of about 110 kDa.

The “knob-in-hole” strategy (see, e.g., PCT Intl. Publ. No. WO 2006/028936) has its plain and ordinary meaning as read in light of the specification and refers to a strategy that may be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”.

As used herein, the term “electrostatic steering” has its plain and ordinary meaning as understood in light of the specification and refer to a mechanism for enhancing the binding rate and affinity of ionic ligands by a distribution of counter charges that evoke an attractive potential field.

As used herein, the term “charge-charge pairing” has its plain and ordinary meaning as understood in light of the specification and refers to electrostatic interactions between two charged particles. These interactions are potentially the strongest noncovalent forces and can extend over greater distances than other noncovalent interactions.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or carrier can refer to a diluent, adjuvant, excipient, or vehicle with which the pharmaceutical formulation is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include sugars, starch, glucose, fructose, lactose, sucrose, maltose, gelatin, malt, rice, flour, chalk, silica gel, sodium stearate, glycerol monostearate, talc, salts, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as glycerol, erythritol, threitol, arabitol, xylitol, ribitol, mannitol, sorbitol, galactitol, fucitol, iditol, inositol, isomalt, maltitol, or lactitol, salt-forming counterions such as sodium, and nonionic surfactants such as TWEEN®, polyethylene glycol (PEG), and PLURONICS®. The formulation, if desired, can also contain minor amounts of wetting, bulking, emulsifying agents, or pH buffering agents. These formulations can take the form of solutions, suspensions, emulsion, sustained release formulations and the like. The formulation should suit the mode of administration.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), tris(hydroxymethyl)aminomethane (Tris), citric acid, ascorbic acid, acetic acid, salts, phosphates, citrates, acetates, succinates, chlorides, bicarbonates, borates, sulfates, sodium chloride, sodium bicarbonate, sodium phosphate, sodium borate, sodium citrate, potassium chloride, potassium phosphate, magnesium sulfate sugars, dextrose, dextran 40, fructose, mannose, lactose, trehalose, galactose, sucrose, sorbitol, mannitol, cellulose, serum, amino acids, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, polysorbate 20, polysorbate 40, polysorbate, 60, polysorbate 80, poloxamer, poloxamer 188, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in the formulation at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “purity” of any given substance, compound, or material as used herein has its plain and ordinary meaning as read in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di, and triethanolamine; amino acids, including glycine, arginine and lysine; guanidine; Nmethylglucosamine; N-methylglucamine; L-glutamine; N-methylpiperazine; morpholine; ethylenediamine; N-benzylphenethylamine; trihydroxymethyl aminoethane.

### Embodiments of the Disclosure

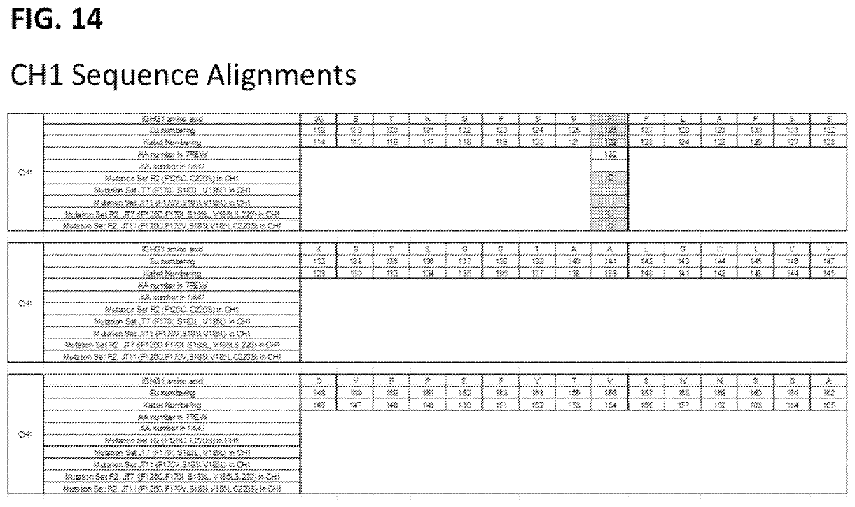

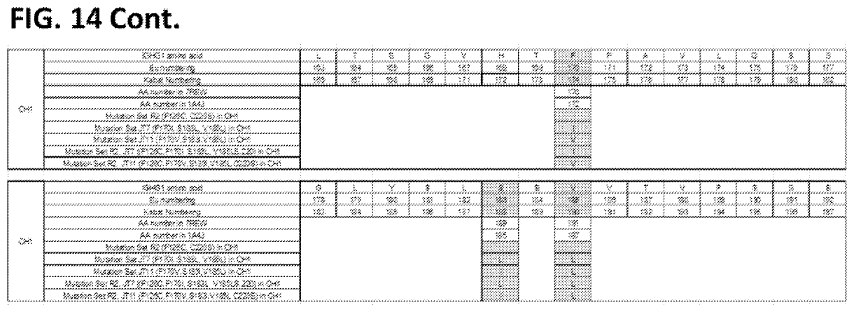

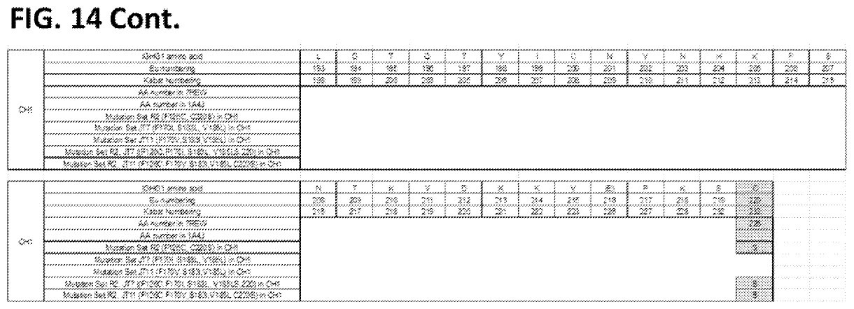

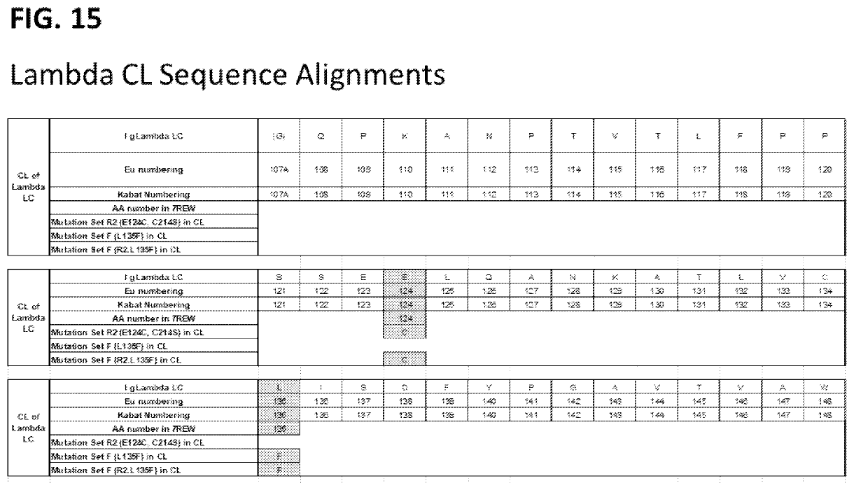

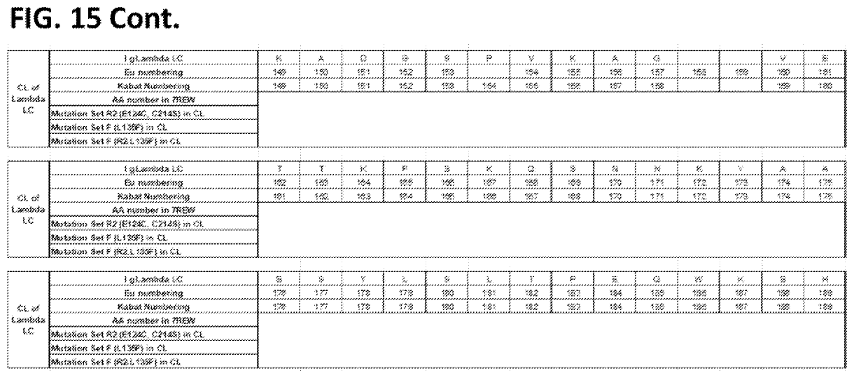

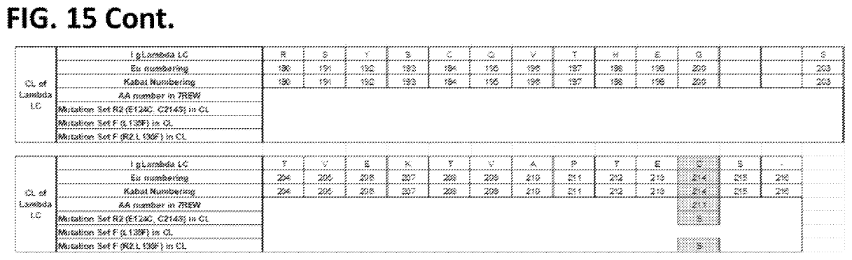

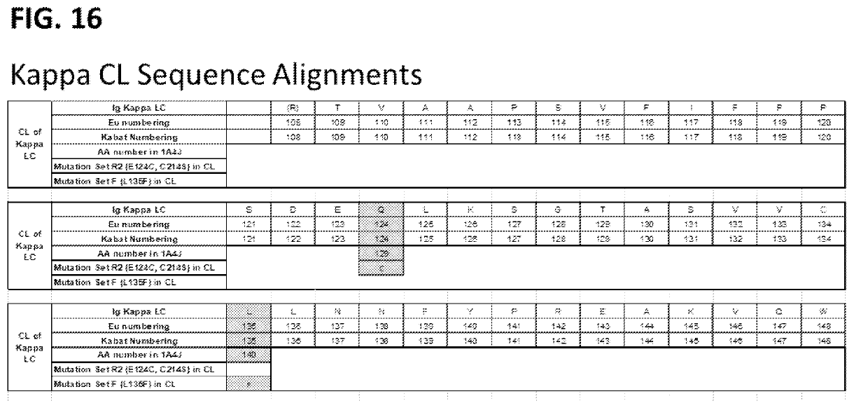

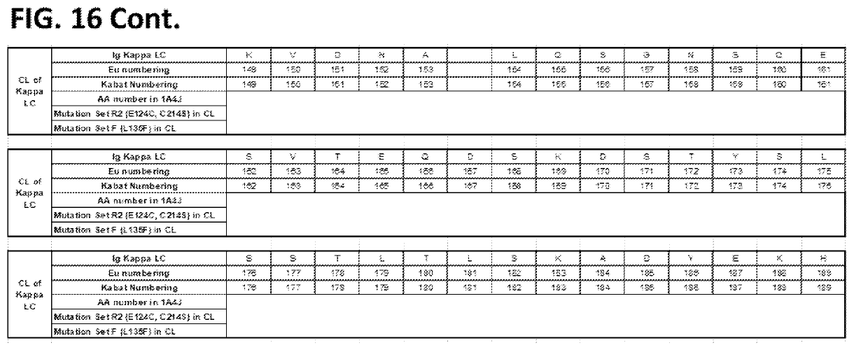

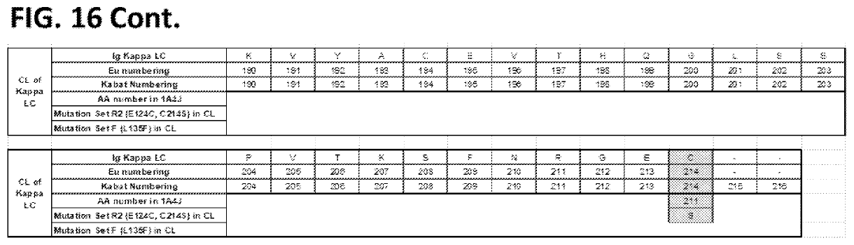

Some embodiments herein relate to Fab(s), antibodies, compositions comprising Fabs and/or antibodies, and methods of producing Fabs and/or antibodies. In some embodiments, residues within a Fab, antibody, or a domain thereof are referenced with respect to the position of the residue within the Fab, antibody, or a domain thereof, according to one or more numbering conventions, for example EU of Kabat numbering. FIG. 14 is a table showing some embodiments of CH1 sequence alignments of wild type and mutant CH1 domains of IGHG1. FIG. 15 is a table showing some embodiments of CL sequence alignments of lambda and mutant CL domains. FIG. 16 is a table showing some embodiments of CL sequence alignments of kappa and mutant CL domains. Unless stated otherwise, all references to specific substitutions at numbered residues utilizes the European Numbering system.

Disclosed herein are embodiments directed to an antigen-binding fragment (Fab).

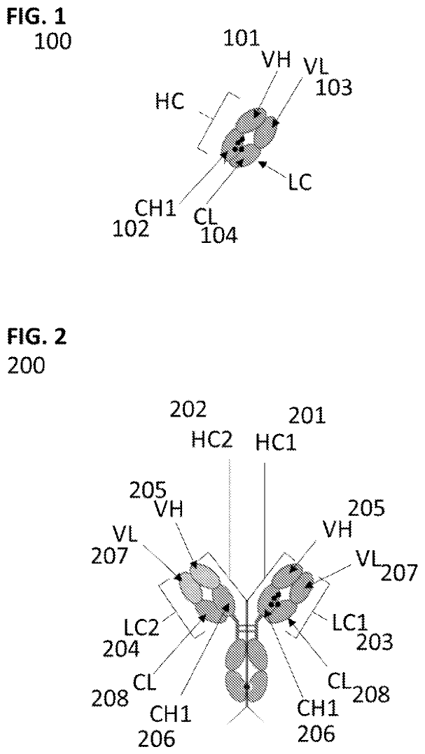

FIG. 1 is an illustration of an embodiment of an antigen-binding fragment

(Fab). FIG. 1, the Fab 100 includes: a heavy chain (HC) variable domain (VH) 101, a heavy chain (HC) constant domain (CH1) of a human IgG 102, a light chain (LC) variable domain (VL) 103, and a light chain (LC) constant domain (CL) of a human IgG 104, wherein the HC CH1 comprises a substitution at F170, S183, and V185 (EU Numbering), and the LC CL comprises a substitution at L135 (EU Numbering), wherein the HC1 does not include any substitution selected from L128F, A141M, A141T, A141I, F170M, F170Y, F170S, F170A, S181I, S181T, S181M, S183A, S183V, and V185A (EU Numbering), and wherein the CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (EU Numbering). In some embodiments, the CH1 consists of substitutions at F170, S183 and V185 (EU Numbering), and wherein the CL consists of a substitution at L135 (EU Numbering). In some embodiments, the HC CH1 substitution at F170 (EU Numbering) is an F170I, or F170V substitution. In some embodiments, the HC CH1 substitution at S183 (EU Numbering) is a S183L or S183I substitution. In some embodiments, the HC CH1 substitution at V185 (EU Numbering) is a V185L substitution. In some embodiments, the LC CL substitution at L135 (EU Numbering) is a L135F substitution. In some embodiments, the HC CH1 comprises an F1701, S183L, and V185L (EU Numbering) substitution, and the LC CL comprises a L135F (EU Numbering) substitution. In some embodiments, the HC CH1 comprises or consists of SEQ ID NO: 15, 19, 23, or 24, and the LC CL comprises or consists of SEQ ID NO: 2, 5, 7, or 8. In some embodiments, the HC CH1 comprises a substitution at F53 and S66, and V68 of SEQ ID NO: 9, and the LC CL comprises a substitution at L29 of SEQ ID NO: 1 or L28 of SEQ ID NO: 6. In some embodiments, the HC CH1 substitution at F53 of SEQ ID NO: 9 is an F53I, or F53V substitution. In some embodiments, the HC CH1 substitution at S66 of SEQ ID NO: 9 is a S66L or S66I substitution. In some embodiments, the HC CH1 substitution at V68 of SEQ ID NO: 9 is a V68L substitution. In some embodiments, the LC CL substitution at L29 of SEQ ID NO: 1 or L28 of SEQ ID NO: 6 is a L29F or L28F substitution. In some embodiments, the Fab is a chimeric or humanized Fab. In some embodiments the Fab is a human Fab.

In some embodiments, the antigen-binding fragment (Fab) comprises a heavy chain (HC) variable domain (VH), a heavy chain (HC) constant domain (CH1) of a human IgG a light chain (LC) variable domain (VL), and a light chain (LC) constant domain (CL) of a human IgG, wherein the HC CH1 comprises a substitution at F126, F170, S183, V185, and C220 (EU Numbering), and the LC CL comprises a substitution at E124, L135, and C214 (lambda) or Q124, L135, and C214 (kappa) (EU Numbering) (in some embodiments the LC CL comprises a substitution at E124, L135, and C214 (lambda); in some embodiments the LC CL comprises a substitution at Q124, L135, and C214 (kappa)), wherein the HC1 does not include any substitution selected from L128F, A141M, A14IT, A141I, F170M, F170Y, F170S, F170A, S181I, S181T, S181M, S183A, S183V, and V185A (EU Numbering), and wherein the CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (EU Numbering). In some embodiments, the CH1 consists of substitutions at F126, F170, S183, V185, and C220 (EU Numbering), and wherein the LC CL consists of a substitution at E124, L135, and C214 (lambda) or Q124, L135, and C214 (kappa) (EU Numbering). In some embodiments the LC CL consists of a substitution at E124, L135, and C214 (lambda). In some embodiments the LC CL consists of a substitution at Q124, L135, and C214 (kappa). In some embodiments, the HC CH1 substitution at F126 (EU Numbering) is an F126C substitution. In some embodiments, the HC CH1 substitution at F170 (EU Numbering) is an F170V or F170I substitution. In some embodiments, the HC CH1 substitution at S183 (EU Numbering) is a S183L, or S183I substitution. In some embodiments, the HC CH1 substitution at V185 (EU Numbering) is a V185L substitution. In some embodiments, the HC CH1 substitution at C220 (EU Numbering) is a C220S substitution. In some embodiments, the LC CL1 substitution at position 124 (EU Numbering) is an E124C (lambda) or Q124C (kappa) substitution. In some embodiments, the LC CL1 substitution at position 124 (EU Numbering) is an E124C (lambda) substitution. In some embodiments, the LC CL1 substitution at position 124 (EU Numbering) is a Q124C (kappa) substitution. In some embodiments, the LC CL1 substitution at L135 (EU Numbering) is a L135F substitution. In some embodiments, the LC CL1 substitution at C214 (EU Numbering) is a C214S substitution. In some embodiments, the HC CH1 comprises or consists of an F126C, F170V, S183I, V185L, and C220S (EU Numbering) substitution, and the LC CL comprises an E124C, L135F, and a C214S (EU Numbering) substitution. In some embodiments, the HC CH1 comprises or consists of an F126C, F170I, S183L, V185L, and C220S (EU Numbering) substitution, and the LC CL comprises or consists of an E124C, L135F, and C214S (lambda) or Q124C, L135F, and C214S (kappa) (EU Numbering) substitution. In some embodiments, the LC CL comprises or consists of an E124C, L135F, and C214S (lambda) (EU Numbering) substitution. In some embodiments, the LC CL comprises or consists of a Q124C, L135F, and C214S (kappa) (EU Numbering) substitution. In some embodiments, the HC CH1 comprises or consists of SEQ ID NO: 15, 19, 23, or 24, and the LC CL comprises or consists of SEQ ID NO: 2, 5, 7, or 8. In some embodiments, the CH1 consists of substitutions at F9, F53, S66, V68, and C103, of SEQ ID NO: 9 and wherein the LC CL consists of a substitution at E18, L29, and C105 of SEQ ID NO: 1. In some embodiments, the CH1 consists of substitutions at F9, F53, S66, V68, and C103, of SEQ ID NO: 9, and wherein the LC CL consists of a substitution at Q17, L28, and C107 of SEQ ID NO: 6. In some embodiments, the HC CH1 substitution at F9 is an F9C substitution. In some embodiments, the HC CH1 substitution at F53 is an F53V or F53I substitution. In some embodiments, the HC CH1 substitution at S66 is a S66L, or S66I substitution. In some embodiments, the HC CH1 substitution at V68 is a V68L substitution. In some embodiments, the HC CH1 substitution at C103 is a C103S substitution. In some embodiments, the LC CL1 substitution at Q17 of SEQ ID NO: 6: is an Q17C substitution. In some embodiments, the LC CL1 substitution at E18 of SEQ ID NO: 1: is an E18C substitution. In some embodiments, the LC CL1 substitution at L28 of SEQ ID NO: 6 is a L28F substitution. In some embodiments, the LC CL1 substitution at L29 of SEQ ID NO: 1 is a L29F substitution. In some embodiments, the LC CL1 substitution at C107 of SEQ ID NO: 6 is a C107S substitution. In some embodiments, the LC CL1 substitution at C105 of SEQ ID NO: 1 is a C105S substitution. In some embodiments, the HC CH1 comprises or consists of an F9C, F53V, S66I, V68L, and C103S substitution, and the LC CL comprises a Q17C, L28F, and a C107S substitution. In some embodiments, the HC CH1 comprises or consists of an F9C, F53V, S66I, V68L, and C103S substitution, and the LC CL comprises an E18C, L29F, and a C105S substitution at a position corresponding to F9, F53, S66, V68, and C103 of SEQ ID NO: 9 and E18, L29, and C105 of SEQ ID NO: 1. In some embodiments, the HC CH1 comprises or consists of an F9C, F53V, S66I, V68L, and C103S substitution, and the LC CL comprises a Q17C, L28F, and C107S substitution at a position corresponding to F9, F53, S66, V68, and C103 of SEQ ID NO: 9 and Q17, L28, and C107 of SEQ ID NO: 6. In some embodiments, the Fab is a chimeric or humanized Fab. In some embodiments the Fab is a human Fab.

In some embodiments, the antigen-binding fragment (Fab) comprises a heavy chain (HC) variable domain (VH), a heavy chain (HC) constant domain (CH1) of a human IgG a light chain (LC) variable domain (VL), and a light chain (LC) constant domain (CL) of a human IgG, wherein the HC CH1 comprises a substitution at F126 and C220 (EU Numbering), and the LC CL comprises a substitution at E124 and C214 (lambda) or Q124 and C214 (kappa) (EU Numbering) (in some the LC CL comprises a substitution at E124 and C214 (lambda); in some embodiments the LC CL comprises a substitution at Q124 and C214 (kappa)); wherein the HC1 does not include any substitution selected from L128F, A141M, A141T, A141I, F170I, F170M, F170Y, F170S, F170A, F170V, S181I, S181T, S181M, S183A, S183L, S1831, S183V, V185L and V185A (EU Numbering); and wherein the CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (European Numbering). In some embodiments, the HC CH1 substitution at F126 (EU Numbering) is an F126C substitution, and the LC CL substitution at E124 (lambda) or Q124 (kappa) (EU Numbering) is an E124C or Q124C substitution. In some embodiments, the LC CL substitution at E124 (lambda) is an E124C substitution. In some embodiments, the LC CL substitution at Q124 (kappa) a Q124C substitution. In some embodiments, the HC CH1 substitution at C220 (EU Numbering) is a C220S substitution. In some embodiments, LC CL substitution at C214 (EU Numbering) is a C214S substitution. In some embodiments, the Fab is a chimeric or humanized Fab. In some embodiments the Fab is a human Fab.

In some embodiments, the antigen-binding fragment (Fab) comprises a heavy chain (HC) variable domain (VH), a heavy chain (HC) constant domain (CH1) of a human IgG a light chain (LC) variable domain (VL), and a light chain (LC) constant domain (CL) of a human IgG, wherein the LC VL framework region (FR) and/or the HC VH framework region (FR) comprises one or more substitutions. In some embodiments, the VL FR comprises a substitution at Q38, and/or G100, and/or G101 (EU Numbering). In some embodiments, the VH FR comprises a substitution at Q39, and/or G44 (EU Numbering). In some embodiments, the VH FR comprises a substitution at Q39, and/or G44 (EU Numbering), and the VL FR comprises a substitution at Q38, and/or G100, and/or G101 (EU Numbering). In some embodiments, the VL FR substitution at Q38 comprises or consists of a substitution selected from the group consisting of a Q38D and a Q38K substitution (EU Numbering). In some embodiments, the VL FR substitution at G100 comprises or consists of a substitution selected from the group consisting of a G100D, a G100K, and a G100C substitution (EU Numbering). In some embodiments, the VL FR substitution at G101 comprises or consists of a substitution selected from the group consisting of G101D and G101K substitution (EU Numbering). In some embodiments, the VH FR substitution at Q39 comprises or consists of a substitution selected from the group consisting of a Q39D and a Q39K substitution (EU Numbering). In some embodiments, the VH FR substitution at G44 comprises or consists of a substitution selected from the group consisting of a G44K, a G44D, and a G44C substitution (EU Numbering). In some embodiments, the substitution of the VH FR comprises or consists of a Q39K substitution, and the substitution of the VL FR comprises or consists of a Q38D substitution. In some embodiments, the substitution of the VH FR comprises or consists of a Q39D substitution, and the substitution of the VL FR comprises or consists of a Q38K substitution. In some embodiments, the substitution of VH FR comprises or consists of a G44K substitution, and the substitution of the VL FR comprises or consists of a G100D substitution. In some embodiments, the substitution of the VH FR comprises or consists of a G44D substitution, and the substitution of the VL FR comprises or consists of a G100K substitution. In some embodiments, the substitution of the VH FR comprises or consists of a G44C substitution, and the substitution of the VL FR comprises or consists of a G100C substitution. In some embodiments, the substitution of the VH FR comprises or consists of a G44K substitution, and the substitution of the VL FR comprises or consists of a G101D substitution. In some embodiments, the substitution of the VH FR comprises or consists of a G44D substitution, and the substitution of the VL FR comprises or consists of a G101K substitution. In some embodiments, the substitution of the HC1 VH FR comprises or consists of a Q39K substitution, the substitution of the LC1 VL FR comprises or consists of a Q38D substitution, the substitution of the HC2 VH FR comprises or consists of a Q39D substitution, and the substitution of the LC2 VL FR comprises or consists of a Q38K substitution. In some embodiments, the substitution of the HC1 VH FR comprises or consists of a Q39D substitution, the substitution of the LC1 VL FR comprises or consists of a Q38K substitution, the substitution of the HC2 VH FR comprises or consists of a Q39K substitution, and the substitution of the LC2 VL FR comprises or consists of a Q38D substitution. In some embodiments, the substitution of the HC1 VH FR comprises or consists of a G44K substitution, the substitution of the LC1 VL FR comprises or consists of a G100D substitution, the substitution of the HC2 VH FR comprises or consists of a G44D substitution, and the substitution of the LC2 VL FR comprises or consists of a G100K substitution. In some embodiments, the substitution of the HC1 VH FR comprises or consists of a G44D substitution, the substitution of the LC1 VL FR comprises or consists of a G100K substitution, the substitution of the HC2 VH FR comprises or consists of a G44K substitution, and the substitution of the LC2 VL FR comprises or consists of a G100D substitution. In some embodiments, the substitution of the HC1 VH FR comprises or consists of a G44C substitution and the substitution of the LC1 VL FR comprises or consists of a G100C substitution. In some embodiments, the substitution of the HC2 VH FR comprises or consists of a G44C substitution and the substitution of the LC1 VL FR comprises or consists of a G100C substitution. In some embodiments, the substitution of HC1 VH FR comprises or consists of a G44K substitution, the substitution of the LC1 VL FR comprises or consists of a G101D substitution, the substitution of the HC2 VH FR comprises or consists of a G44D substitution, and the substitution of the LC2 VL FR comprises or consists of a G101K substitution. In some embodiments, the substitution of HC1 VH FR comprises or consists of a G44D substitution, the substitution of the LC1 VL FR comprises or consists of a G101K substitution, the substitution of the HC2 VH FR comprises or consists of a G44K substitution, and the substitution of the LC2 VL FR comprises or consists of a G101D substitution.

Disclosed herein are antibodies. In some embodiments, the antibodies comprise one or more Fab fragments as illustrated in FIG. 1 and discussed above an elsewhere herein. FIG. 2 is an illustration showing an embodiment of an antibody. Some embodiments herein relate to an antibody 200 or a fragment thereof comprising: An antibody or a fragment thereof comprising: a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG, wherein the HC1 CH1 comprises or consists of a substitution at F170 and S183, and optionally a substitution at V185 (EU Numbering), and the LC1 CL comprises or consists of a substitution at L135 (EU Numbering), wherein the HC1 CH1 does not include any substitution selected from L128F, A141M, A141T, A141I, F170M, F170Y, F170S, F170A, S181I, S181T, S181M, S183A, S183V, and V185A (EU Numbering), and wherein the LC1 CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (EU Numbering). In some embodiments, the fragment comprises an antigen binding fragment. In some embodiments, the antibody is a human IgG. In some embodiments, the antibody is a human IgG1, IgG2, IgG3, or IgG4, antibody. In some embodiments, the antibody is a chimeric antibody. In some embodiments, the antibody is a humanized antibody. In some embodiments, the antibody is a chimeric or humanized IgG1, IgG2, IgG3, or IgG4, antibody. In some embodiments, the HC1's CH1 consists of substitutions at F170, S183 and V185 (EU Numbering), and wherein the LC1's CL consists of a substitution at L135 (EU Numbering), In some embodiments, the HC2 CH1 comprises or consists of SEQ ID NO: 9, and the LC2 CL comprises or consists of SEQ ID NO: 1 or 6. In some embodiments, the HC1 CH1 substitution at F170 (EU Numbering) is an F170I, or F170V substitution. In some embodiments, the HC1 CH1 substitution at S183 (EU Numbering) is a S183L or S183I substitution. In some embodiments, the HC1 CH1 substitution at V185 (EU Numbering) is a V185L substitution. In some embodiments, the LC1 CL1 substitution at L135 (EU Numbering) is a L135F substitution. In some embodiments, the HC1 CH1 comprises or consists of an F170V, S1831 and V185L (EU Numbering) substitution, and the LC1 CL comprises or consists of a L135F (EU Numbering) substitution. In some embodiments, the HC1 CH1 comprises or consists of an F170I, S183L, and V185L (EU Numbering) substitution, and the LC1 CL comprises or consists of f a L135F (EU Numbering) substitution. In some embodiments, the HC CH1 comprises a substitution at F53 and S66, and V68 of SEQ ID NO: 9, and the LC CL comprises a substitution at L29 of SEQ ID NO: 1 or L28 of SEQ ID NO: 6. In some embodiments, the HC CH1 substitution at F53 of SEQ ID NO: 9 is an F531, or F53V substitution. In some embodiments, the HC CH1 substitution at S66 of SEQ ID NO: 9 is a S66L or S66I substitution. In some embodiments, the HC CH1 substitution at V68 of SEQ ID NO: 9 is a V68L substitution. In some embodiments, the LC CL substitution at L29 of SEQ ID NO: 1 or L28 of SEQ ID NO: 6 is a L29F or L28F substitution.

In some embodiments, the antibody or a fragment thereof comprises a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG, wherein the HC1 CH1 comprises or consists of a substitution at F126, F170, S183, V185, and C220 (EU Numbering), and the LC1 CL comprises or consists of a substitution at E124, L135, and C214 (lambda) or Q124, L135, and C214 (kappa) (EU Numbering) (in some embodiments the LC CL comprises or consists of a substitution at E124, L135, and C214 (lambda); in some embodiments the LC CL comprises or consists of a substitution at Q124, L135, and C214 (kappa)), wherein the HC1 CH1 does not include any substitution selected from L128F, A141M, A141T, A141I, F170M, F170Y, F170S, F170A, S181I, S18IT, S181M, S183A, S183V, and V185A (EU Numbering), and wherein the LC1 CL does not include any substitution selected from F116A, F118V, S13IT, S131D, V133A, V133I, L13SY, L135V, S162A, S162M, T164S, S174A, S176M, S176A, S176T, S176F, T178L, T178V, and T178I (EU Numbering). In some embodiments, the fragment comprises an antigen binding fragment. In some embodiments, the antibody is a human IgG. In some embodiments, the antibody is a human IgG1, IgG2, IgG3, or IgG4, antibody. In some embodiments, the antibody is a chimeric antibody. In some embodiments, the antibody is a humanized antibody. In some embodiments, the antibody is a chimeric or humanized IgG1, IgG2, IgG3, or IgG4, antibody. In some embodiments, the HC CH1 substitution at F126 (EU Numbering) is an F126C substitution. In some embodiments, the HC CH1 substitution at F170 (EU Numbering) is an F170V or F1701 substitution. In some embodiments, the HC CH1 substitution at S183 (EU Numbering) is a S183L, or S183I substitution. In some embodiments, the HC CH1 substitution at V185 (EU Numbering) is a V185L substitution. In some embodiments, the HC CH1 substitution at C220 (EU Numbering) is a C220S substitution. In some embodiments, the LC CL1 substitution at position 124 (EU Numbering) is an E124C (lambda) or Q124C (kappa) substitution. In some embodiments, the LC CL1 substitution at position 124 (EU Numbering) is an E124C (lambda) substitution. In some embodiments, the LC CL1 substitution at position 124 (EU Numbering) is a Q124C (kappa) substitution. In some embodiments, the LC CL1 substitution at L135 (EU Numbering) is a L135F substitution. In some embodiments, the LC CL1 substitution at C214 (EU Numbering) is a C214S substitution. In some embodiments, the HC CH1 comprises or consists of an F126C, F170V, S183I, V185L, and C220S (EU Numbering) substitution, and the LC CL comprises an E124C, L135F, and C214S (lambda) or Q124C, L135F, and C214S (kappa) (EU Numbering) substitution. In some embodiments, the LC CL comprises an E124C, L135F, and C214S (lambda) (EU Numbering) substitution. In some embodiments, the LC CL comprises a Q124C, L135F, and C214S (kappa) (EU Numbering) substitution. In some embodiments, the HC CH1 comprises or consists of an F126C, F170I, S183L, V185L, and C220S (EU Numbering) substitution, and the LC CL comprises an E124C, L135F, and C214S (lambda) or Q124C, L135F, and C214S (kappa) (EU Numbering) substitution. In some embodiments, the LC CL comprises an E124C, L135F, and C214S (lambda) (EU Numbering) substitution. In some embodiments, the LC CL comprises a Q124C, L135F, and C214S (kappa) (EU Numbering) substitution. In some embodiments, the CH1 consists of substitutions at F9, F53, S66, V68, and C103, of SEQ ID NO: 9 and wherein the LC CL consists of a substitution at E18, L29, and C105 of SEQ ID NO: 1. In some embodiments, the CH1 consists of substitutions at F9, F53, S66, V68, and C103 of SEQ ID NO: 9, and wherein the LC CL consists of a substitution at Q17, L28, and C107 of SEQ ID NO: 6. In some embodiments, the HC CH1 substitution at F9 is an F9C substitution. In some embodiments, the HC CH1 substitution at F53 is an F53V or F53I substitution. In some embodiments, the HC CH1 substitution at S66 is a S66L, or S66I substitution. In some embodiments, the HC CH1 substitution at V68 is a V68L substitution. In some embodiments, the HC CH1 substitution at C103 is a C103S substitution. In some embodiments, the LC CL1 substitution at Q17 of SEQ ID NO: 6: is an Q17C substitution. In some embodiments, the LC CL1 substitution at E18 of SEQ ID NO: 1: is an E18C substitution. In some embodiments, the LC CL1 substitution at L28 of SEQ ID NO: 6 is a L28F substitution. In some embodiments, the LC CL1 substitution at L29 of SEQ ID NO: 1 is a L29F substitution. In some embodiments, the LC CL1 substitution at C107 of SEQ ID NO: 6 is a C107S substitution. In some embodiments, the LC CL1 substitution at C105 of SEQ ID NO: 1 is a C105S substitution. In some embodiments, the HC CH1 comprises or consists of an F9C, F53V, S66I, V68L, and C103S substitution, and the LC CL comprises a Q17C, L28F, and a C107S (AA sequence numbering) substitution. In some embodiments, the HC CH1 comprises or consists of an F9C, F53V, S66I, V68L, and C103S substitution, and the LC CL comprises an E18C, L29F, and a C105S substitution at a position corresponding to F9, F53, S66, V68, and C103 of SEQ ID NO: 9 and E18, L29, and C105 of SEQ ID NO: 1. In some embodiments, the HC CH1 comprises or consists of an F9C, F53V, S66I, V68L, and C103S substitution, and the LC CL comprises a Q17C, L28F, and a C107S substitution at a position corresponding to F9, F53, S66, V68, and C103 of SEQ ID NO: 9 and Q17, L28, and C107 of SEQ ID NO: 6.

In some embodiments, the antibody or a fragment thereof comprises a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG, wherein the LC1 and/or LC2 VL framework region (FR) and/or the HC1 and/or HC2 VH framework region (FR) comprises one or more substitutions. In some embodiments, the LC1 and/or LC2 VL FR comprises a substitution at Q38, and/or G100, and/or G101 (EU Numbering). In some embodiments, the HC1 and/or HC2 VH FR comprises a substitution at Q39, and/or G44 (EU Numbering). In some embodiments, the HC1 and/or HC2 VH FR comprises a substitution at Q39, and/or G44 (EU Numbering), and the LC1 and/or LC2 VL FR comprises a substitution at Q38, and/or G100, and/or G101 (EU Numbering). In some embodiments, the LC1 and/or LC2 VL FR substitution at Q38 comprises or consists of a substitution selected from the group consisting of a Q38D and a Q38K substitution (EU Numbering). In some embodiments, the LC1 and/or LC2 VL FR substitution at G100 comprises or consists of a substitution selected from the group consisting of a G100D, a G100K, and a G100C substitution (EU Numbering). In some embodiments, the LC1 and/or LC2 VL FR substitution at G101 comprises or consists of a substitution selected from the group consisting of G101D and G101K substitution (EU Numbering). In some embodiments, the HC1 and/or HC2 VH FR substitution at Q39 comprises or consists of a substitution selected from the group consisting of a Q39D and a Q39K substitution (EU Numbering). In some embodiments, the HC1 and/or HC2 VH FR substitution at G44 comprises or consists of a substitution selected from the group consisting of a G44K, a G44D, and a G44C substitution (EU Numbering). In some embodiments, the substitution of the HC1 and/or HC2 VH FR comprises or consists of a Q39K substitution, and the substitution of the LC1 and/or LC2 VL FR comprises or consists of a Q38D substitution. In some embodiments, the substitution of the HC1 and/or HC2 VH FR comprises or consists of a Q39D substitution, and the substitution of the LC1 and/or LC2 VL FR comprises or consists of a Q38K substitution. In some embodiments, the substitution of HC1 and/or HC2 VH FR comprises or consists of a G44K substitution, and the substitution of the LC1 and/or LC2 VL FR comprises or consists of a G100D substitution. In some embodiments, the substitution of the HC1 and/or HC2 VH FR comprises or consists of a G44D substitution, and the substitution of the LC1 and/or LC2 VL FR comprises or consists of a G100K substitution. In some embodiments, the substitution of the HC1 and/or HC2 VH FR comprises or consists of a G44C substitution, and the substitution of the LC1 and/or LC2 VL FR comprises or consists of a G100C substitution. In some embodiments, the substitution of the HC1 and/or HC2 VH FR comprises or consists of a G44K substitution, and the substitution of the LC1 and/or LC2 VL FR comprises or consists of a G101D substitution. In some embodiments, the substitution of the HC1 and/or HC2 VH FR comprises or consists of a G44D substitution, and the substitution of the LC1 and/or LC2 VL FR comprises or consists of a G101K substitution. In some embodiments, the substitution of the HC1 VH FR comprises or consists of a Q39K substitution, the substitution of the LC1 VL FR comprises or consists of a Q38D substitution, the substitution of the HC2 VH FR comprises or consists of a Q39D substitution, and the substitution of the LC2 VL FR comprises or consists of a Q38K substitution. In some embodiments, the substitution of the HC1 VH FR comprises or consists of a Q39D substitution, the substitution of the LC1 VL FR comprises or consists of a Q38K substitution, the substitution of the HC2 VH FR comprises or consists of a Q39K substitution, and the substitution of the LC2 VL FR comprises or consists of a Q38D substitution. In some embodiments, the substitution of the HC1 VH FR comprises or consists of a G44K substitution, the substitution of the LC1 VL FR comprises or consists of a G100D substitution, the substitution of the HC2 VH FR comprises or consists of a G44D substitution, and the substitution of the LC2 VL FR comprises or consists of a G100K substitution. In some embodiments, the substitution of the HC1 VH FR comprises or consists of a G44D substitution, the substitution of the LC1 VL FR comprises or consists of a G100K substitution, the substitution of the HC2 VH FR comprises or consists of a G44K substitution, and the substitution of the LC2 VL FR comprises or consists of a G100D substitution. In some embodiments, the substitution of the HC1 VH FR comprises or consists of a G44C substitution and the substitution of the LC1 VL FR comprises or consists of a G100C substitution. In some embodiments, the substitution of the HC2 VH FR comprises or consists of a G44C substitution and the substitution of the LC1 VL FR comprises or consists of a G100C substitution. In some embodiments, the substitution of HC1 VH FR comprises or consists of a G44K substitution, the substitution of the LC1 VL FR comprises or consists of a G101D substitution, the substitution of the HC2 VH FR comprises or consists of a G44D substitution, and the substitution of the LC2 VL FR comprises or consists of a G101K substitution. In some embodiments, the substitution of HC1 VH FR comprises or consists of a G44D substitution, the substitution of the LC1 VL FR comprises or consists of a G101K substitution, the substitution of the HC2 VH FR comprises or consists of a G44K substitution, and the substitution of the LC2 VL FR comprises or consists of a G101D substitution.

In some embodiments, the antibody or a fragment thereof comprises a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG, wherein the LC1 and/or LC2 VL framework region (FR) and/or the HC1 and/or HC2 VH framework region (FR) comprises one or more substitutions. In some embodiments, the HC1 or HC2 VH comprises or consists of any one of SEQ ID NO: 53-64. In some embodiments, the LC1 or LC2 VL comprises or consists of any one of SEQ ID NO: 65-80. In some embodiments, the heavy chain and light chain of the HC1/LC1 pair and the heavy chain and light chain of the HC2/LC2 pair each comprise or consist of the following sequence, respectively: SEQ ID NOs: 30, 43, 35 and 52; or SEQ ID NOs: 35, 52, 30 and 43. In some embodiments, the heavy chain and light chain of the HC1/LC1 pair and the heavy chain and light chain of the HC2/LC2 pair each comprise or consist of the following sequence, respectively: SEQ ID NOs: 29, 44, 36 and 51; or SEQ ID NOs: 36, 51, 29 and 44. In some embodiments, the heavy chain and light chain of the HC1/LC1 pair and the heavy chain and light chain of the HC2/LC2 pair each comprise or consist of the following sequence, respectively: SEQ ID NOs: 28, 39, 33 and 48; or SEQ ID NOs: 33, 48, 28 and 39. In some embodiments, the heavy chain and light chain of the HC1/LC1 pair and the heavy chain and light chain of the HC2/LC2 pair each comprise or consist of the following sequence, respectively: SEQ ID NOs: 27, 40, 34 and 47; or SEQ ID NOs: 34, 47, 27 and 40. In some embodiments, the heavy chain and light chain of the HC1/LC1 pair and the heavy chain and light chain of the HC2/LC2 pair each comprise or consist of the following sequence, respectively: SEQ ID NOs: 26, 38, 31 and 45; or SEQ ID NOs: 31, 45, 26 and 38. In some embodiments, the heavy chain and light chain of the HC1/LC1 pair and the heavy chain and light chain of the HC2/LC2 pair each comprise or consist of the following sequence, respectively: SEQ ID NOs: 25, 37, 32 and 46; or SEQ ID NOs: 32, 46, 25 and 37. In some embodiments, the heavy chain and light chain of the HC1/LC1 pair and the heavy chain and light chain of the HC2/LC2 pair each comprise or consist of the following sequence, respectively: SEQ ID NOs: 28, 41, 33 and 50; or SEQ ID NOs: 33, 50, 28 and 41. In some embodiments, the heavy chain and light chain of the HC1/LC1 pair and the heavy chain and light chain of the HC2/LC2 pair each comprise or consist of the following sequence, respectively: SEQ ID NOs: 27, 42, 34 and 49; or SEQ ID NOs: 34, 49, 27 and 42. In some embodiments, the HC1 comprises or consists of SEQ ID NO: 30, the LC1 comprises or consists of SEQ ID NO: 43, the HC2 comprises or consists of SEQ ID NO: 35, and the LC2 comprises or consists of SEQ ID NO: 52. In some embodiments, the HC1 comprises or consists of SEQ ID NO: 29, the LC1 comprises or consists of SEQ ID NO: 44, the HC2 comprises or consists of SEQ ID NO: 36, and the LC2 comprises or consists of SEQ ID NO: 51. In some embodiments, the HC1 comprises or consists of SEQ ID NO: 28, the LC1 comprises or consists of SEQ ID NO: 39, the HC2 comprises or consists of SEQ ID NO: 33, and the LC2 comprises or consists of SEQ ID NO: 48. In some embodiments, the HC1 comprises or consists of SEQ ID NO: 27, the LC1 comprises or consists of SEQ ID NO: 40, the HC2 comprises or consists of SEQ ID NO: 34, and the LC2 comprises or consists of SEQ ID NO: 47. In some embodiments, the HC1 comprises or consists of SEQ ID NO: 26, the LC1 comprises or consists of SEQ ID NO: 38, the HC2 comprises or consists of SEQ ID NO: 31, and the LC2 comprises or consists of SEQ ID NO: 45. In some embodiments, the HC1 comprises or consists of SEQ ID NO: 25, the LC1 comprises or consists of SEQ ID NO: 37, the HC2 comprises or consists of SEQ ID NO: 32, and the LC2 comprises or consists of SEQ ID NO: 46. In some embodiments, the HC1 comprises or consists of SEQ ID NO: 28, the LC1 comprises or consists of SEQ ID NO: 39, the HC2 comprises or consists of SEQ ID NO: 33, and the LC2 comprises or consists of SEQ ID NO: 50. In some embodiments, the HC1 comprises or consists of SEQ ID NO: 27, the LC1 comprises or consists of SEQ ID NO: 40, the HC2 comprises or consists of SEQ ID NO: 34, and the LC2 comprises or consists of SEQ ID NO: 49. In some embodiments, the HC1 comprises or consists of SEQ ID NO: 35, the LC1 comprises or consists of SEQ ID NO: 52, the HC2 comprises or consists of SEQ ID NO: 30, and the LC2 comprises or consists of SEQ ID NO: 43. In some embodiments, the HC1 comprises or consists of SEQ ID NO: 36, the LC1 comprises or consists of SEQ ID NO: 51, the HC2 comprises or consists of SEQ ID NO: 29, and the LC2 comprises or consists of SEQ ID NO: 44. In some embodiments, the HC1 comprises or consists of SEQ ID NO: 33, the LC1 comprises or consists of SEQ ID NO: 48, the HC2 comprises or consists of SEQ ID NO: 28, and the LC2 comprises or consists of SEQ ID NO: 39. In some embodiments, the HC1 comprises or consists of SEQ ID NO: 34, the LC1 comprises or consists of SEQ ID NO: 47, the HC2 comprises or consists of SEQ ID NO: 27, and the LC2 comprises or consists of SEQ ID NO: 40. In some embodiments, the HC1 comprises or consists of SEQ ID NO: 31, the LC1 comprises or consists of SEQ ID NO: 45, the HC2 comprises or consists of SEQ ID NO: 26, and the LC2 comprises or consists of SEQ ID NO: 38. In some embodiments, the HC1 comprises or consists of SEQ ID NO: 32, the LC1 comprises or consists of SEQ ID NO: 46, the HC2 comprises or consists of SEQ ID NO: 25, and the LC2 comprises or consists of SEQ ID NO: 37. In some embodiments, the HC1 comprises or consists of SEQ ID NO: 33, the LC1 comprises or consists of SEQ ID NO: 50, the HC2 comprises or consists of SEQ ID NO: 28, and the LC2 comprises or consists of SEQ ID NO: 41. In some embodiments, the HC1 comprises or consists of SEQ ID NO: 34, the LC1 comprises or consists of SEQ ID NO: 49, the HC2 comprises or consists of SEQ ID NO: 27, and the LC2 comprises or consists of SEQ ID NO: 42.

In some embodiments, the antibodies or fragments thereof disclosed herein comprise two heavy chain/light chain pairs, wherein the first pair is HC1/LC1, and the second pair is HC2/LC2. In some embodiments, the antibodies or fragments thereof disclosed herein further comprise an HC1 and HC2 pair. In some embodiments, the HC1 and HC2 are paired through charge-charge interactions. In some embodiments, each HC1 and HC2 further comprises a heavy chain constant domain (CH3), and wherein the CH3 domains of HC1 and HC2 together comprise a knob-into-hole construct. In some embodiments, the HC1 CH3 comprises a knob and the HC2 CH3 comprises a hole. In some embodiments, the HC1 CH3 comprises a hole and the HC2 CH3 comprises a knob. In some embodiments, the LC1 and/or LC2 is a kappa light chain. In some embodiments, the LC1 and/or LC2 is a lambda light chain. In some embodiments, the HC CH1 comprises or consists of SEQ ID NO: 15, 19, 23, or 24, and the LC CL comprises or consists of SEQ ID NO: 2, 5, 7, or 8.

In some embodiments, the antibodies or fragments thereof disclosed herein comprise a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG, wherein the HC1 CH1 comprises or consists of a substitution at F126 and C220 (EU Numbering), the LC1 CL comprises or consists of a substitution at position 124 (E124 for lambda and Q124 for kappa), and C214 (EU Numbering), the HC2 CH1 comprises or consists of a substitution at F170, S183, and V185 (EU Numbering), and the LC2 CL comprises or consists of a substitution L135 (EU Numbering), wherein the HC1 CH1 and/or HC2 CH1 does not include any substitution selected from L128F, A141M, A14IT, A141I, F170M, F170Y, F170S, F170A, S181I, S18IT, S181M, S183A, S183V, and V185A (EU Numbering), and wherein the LC1 CL and/or LC2 CL does not include any substitution selected from F116A, F118V, S131T, S13ID, V133A, V133I, L135Y, L135V, S162A, S162M, T164S, S174A, S176M, S176A, S176T, S176F, T178L, T178V, and T178I (EU Numbering). In some embodiments, the antibody is a human IgG. In some embodiments, the antibody is a human IgG1, IgG2, IgG3, or IgG4, antibody. In some embodiments, the antibody is a chimeric antibody. In some embodiments, the antibody is a humanized antibody. In some embodiments, the antibody is a chimeric or humanized IgG1, IgG2, IgG3, or IgG4, antibody. In some embodiments, the HC1 CH1 substitution at F126 (EU Numbering) is an F126C substitution, and the LC1 CL substitution at position 124 (EU Numbering) is an E124C (lambda) or Q124C (kappa) substitution. In some embodiments, the LC CL substitution at E124 (lambda) is an E124C substitution. In some embodiments, the LC CL substitution at Q124 (kappa) a Q124C substitution. In some embodiments, the HC CH1 substitution at C220 (EU Numbering) is a C220S substitution. In some embodiments, the LC CL substitution at C214 (EU Numbering) is a C214S substitution. In some embodiments, the CH1 consists of substitutions at F9, F53, S66, V68, and C103, of SEQ ID NO: 9 and wherein the LC CL consists of a substitution at E18, L29, and C105 of SEQ ID NO: 1. In some embodiments, the CH1 consists of substitutions at F9, F53, S66, V68, and C103, of SEQ ID NO: 9, and wherein the LC CL consists of a substitution at Q17, L28, and C107 of SEQ ID NO: 6. In some embodiments, the HC CH1 substitution at F9 is an F9C substitution. In some embodiments, the HC CH1 substitution at F53 is an F53V or F53I substitution. In some embodiments, the HC CH1 substitution at S66 is a S66L, or S66I substitution. In some embodiments, the HC CH1 substitution at V68 is a V68L substitution. In some embodiments, the HC CH1 substitution at C103 is a C103S substitution. In some embodiments, the LC CL1 substitution at Q17 of SEQ ID NO: 6; is an Q17C substitution. In some embodiments, the LC CL1 substitution at E18 of SEQ ID NO: 1: is an E18C substitution. In some embodiments, the LC CL1 substitution at L28 of SEQ ID NO: 6 is a L28F substitution. In some embodiments, the LC CL1 substitution at L29 of SEQ ID NO: 1 is a L29F substitution. In some embodiments, the LC CL1 substitution at C107 of SEQ ID NO: 6 is a C107S substitution. In some embodiments, the LC CL1 substitution at C105 of SEQ ID NO: 1 is a C105S substitution. In some embodiments, the HC CH1 comprises or consists of an F9C, F53V, S66I, V68L, and C103S substitution, and the LC CL comprises a Q17C, L28F, and a C107S substitution. In some embodiments, the HC CH1 comprises or consists of an F9C, F53V, S66I, V68L, and C103S substitution, and the LC CL comprises an E18C, L29F, and a C105S substitution at a position corresponding to F9, F53, S66, V68, and C103 of SEQ ID NO: 9 and E18, L29, and C105 of SEQ ID NO: 1. In some embodiments, the HC CH1 comprises or consists of an F9C, F53V, S66I, V68L, and C103S substitution, and the LC CL comprises a Q17C, L28F, and a C107S substitution at a position corresponding to F9, F53, S66, V68, and C103 of SEQ ID NO: 9 and Q17, L28, and C107 of SEQ ID NO: 6.

Some embodiments herein are directed to a composition comprising one or more polynucleotides encoding a Fab and/or antibody comprising: a heavy chain (HC) variable domain (VH) 101, a heavy chain (HC) constant domain (CH1) of a human IgG 102, a light chain (LC) variable domain (VL) 103, and a light chain (LC) constant domain (CL) of a human IgG 104, wherein the HC CH1 comprises or consists of a substitution at F170 and S183, and optionally a substitution at V185 (EU Numbering), and the LC CL comprises or consists of a substitution at L135 (EU Numbering), wherein the HC1 does not include any substitution selected from L128F, A141M, A141T, A141I, F170M, F170Y, F170S, F170A, S181I, S181T, S181M, S183A, S183V, and V185A (EU Numbering), and wherein the CL does not include any substitution selected from F116A, F118V, S13IT, S131D, V133A, V133I, L135Y, L135V, S162A, S162M, T164S, S174A, S176M, S176A, S176T, S176F, T178L, T178V, and T178I (EU Numbering). In some embodiments, the polynucleotide encodes a human IgG. In some embodiments, the polynucleotide encodes a human IgG1, IgG2, IgG3, or IgG4, antibody. In some embodiments, the polynucleotide encodes a chimeric antibody. In some embodiments, the polynucleotide encodes a humanized antibody. In some embodiments, the polynucleotide encodes a chimeric or humanized IgG1, IgG2, IgG3, or IgG4, antibody. In some embodiments, the CH1 consists of substitutions at F170 and S183 (EU Numbering), or at F170, S183 and V185 (EU Numbering), and wherein the CL consists of a substitution at L135 (EU Numbering). In some embodiments, V185 of CH1 is substituted. In some embodiments, the HC CH1 substitution at F170 (EU Numbering) is an F170I, or F170V substitution. In some embodiments, the HC CH1 substitution at S183 (EU Numbering) is a S183L or S183I substitution. In some embodiments, the HC CH1 substitution at V185 (EU Numbering) is a V185L substitution. In some embodiments, the LC CL1 substitution at L135 (EU Numbering) is a L135F substitution. In some embodiments, the HC CH1 comprises or consists of an F170V, S183I, and V185L (EU Numbering) substitution, and the LC CL comprises or consists of a L135F (EU Numbering) substitution. In some embodiments, the HC CH1 comprises or consists of an F170I, S183L, and V185L (EU Numbering) substitution, and the LC CL comprises or consists of a L135F (EU Numbering) substitution. In some embodiments, the HC CH1 comprises or consists of SEQ ID NO: 15, 19, 23, or 24, and the LC CL comprises or consists of SEQ ID NO: 2, 5, 7, or 8. In some embodiments, the HC CH1 comprises a substitution at F53 and S66, and V68 of SEQ ID NO: 9, and the LC CL comprises a substitution at L29 of SEQ ID NO: 1 or L28 of SEQ ID NO: 6. In some embodiments, the HC CH1 substitution at F53 of SEQ ID NO: 9 is an F53I, or F53V substitution. In some embodiments, the HC CH1 substitution at S66 of SEQ ID NO: 9 is a S66L or S66I substitution. In some embodiments, the HC CH1 substitution at V68 of SEQ ID NO: 9 is a V68L substitution. In some embodiments, the LC CL substitution at L29 of SEQ ID NO: 1 or L28 of SEQ ID NO: 6 is a L29F or L28F substitution.

Some embodiments herein are directed to a composition comprising one or more polynucleotides encoding a Fab and/or antibody comprising: a heavy chain (HC) variable domain (VH), a heavy chain (HC) constant domain (CH1) of a human IgG a light chain (LC) variable domain (VL), and a light chain (LC) constant domain (CL) of a human IgG, wherein the HC CH1 comprises or consists of a substitution at F126, F170, S183, V185, and C220 (EU Numbering), and the LC CL comprises or consists of a substitution at E124, L135, and C214 (lambda) or Q124, L135, and C214 (kappa) (EU Numbering) (in some embodiments the LC CL comprises or consists of a substitution at E124, L135, and C214 (lambda); in some embodiments the LC CL comprises or consists of a substitution at Q124, L135, and C214 (kappa)), wherein the HC1 does not include any substitution selected from L128F, A141M, A141T, A141I, F170M, F170Y, F170S, F170A, S181I, S18IT, S181M, S183A, S183V, and V185A (EU Numbering), and wherein the CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (EU Numbering). In some embodiments, the composition comprises a human IgG. In some embodiments, the composition comprises a human IgG1, IgG2, IgG3, or IgG4, antibody. In some embodiments, the composition comprises a chimeric antibody. In some embodiments, the composition comprises a humanized antibody. In some embodiments, the composition comprises a chimeric or humanized IgG1, IgG2, IgG3, or IgG4, antibody. In some embodiments, the CH1 comprises or consists of substitutions at F170 and S183 (EU Numbering), or at F170, S183 and V185 (EU Numbering), and wherein the CL comprises or consists of a substitution at L135 (EU Numbering). In some embodiments, the HC CH1 substitution at F126 (EU Numbering) is an F126C substitution. In some embodiments, the HC CH1 substitution at F170 (EU Numbering) is an F170V or F1701 substitution. In some embodiments, the HC CH1 substitution at S183 (EU Numbering) is a S183L, or S183I substitution. In some embodiments, the HC CH1 substitution at V185 (EU Numbering) is a V185L substitution. In some embodiments, the HC CH1 substitution at C220 (EU Numbering) is a C220S substitution. In some embodiments, the LC CL1 substitution at position 124 (EU Numbering) is an E124C (lambda) or Q124C (kappa) substitution. In some embodiments, the LC CL1 substitution at position 124 (EU Numbering) is an E124C (lambda) substitution. In some embodiments, the LC CL1 substitution at position 124 (EU Numbering) is a Q124C (kappa) substitution. In some embodiments, the LC CL1 substitution at L135 (EU Numbering) is a L135F substitution. In some embodiments, the LC CL1 substitution at C214 (EU Numbering) is a C214S substitution. In some embodiments, the HC CH1 comprises or consists of an F126C, F170V, S183I, V185L, and C220S (EU Numbering) substitution, and the LC CL comprises or consists of an E124C, L13SF, and C214S (lambda) or Q124C, L135F, and C214S (kappa) (EU Numbering) substitution. In some embodiments, the HC CH1 comprises or consists of an F126C, F170I, S183L, V185L, and C220S (EU Numbering) substitution, and the LC CL comprises or consists of an E124C, L135F, and C214S (lambda) or Q124C, L135F, and C214S (kappa) (EU Numbering) substitution. In some embodiments, the LC CL comprises or consists of an E124C, L135F, and C214S (lambda) (EU Numbering) substitution. In some embodiments, the LC CL comprises or consists of a Q124C, L13SF, and C214S (kappa) (EU Numbering) substitution. In some embodiments, the HC CH1 comprises or consists of SEQ ID NO: 15, 19,1 23, or 24, and the LC CL comprises or consists of SEQ ID NO: 2, 5, 7, or 8. In some embodiments, the CH1 consists of substitutions at F9, F53, S66, V68, and C103, of SEQ ID NO: 9 and wherein the LC CL consists of a substitution at E18, L29, and C105 of SEQ ID NO: 1. In some embodiments, the CH1 consists of substitutions at F9, F53, S66, V68, and C103, of SEQ ID NO: 9, and wherein the LC CL consists of a substitution at Q17, L28, and C107 of SEQ ID NO: 6. In some embodiments, the HC CH1 substitution at F9 is an F9C substitution. In some embodiments, the HC CH1 substitution at F53 is an F53V or F53I substitution. In some embodiments, the HC CH1 substitution at S66 is a S66L, or S66I substitution. In some embodiments, the HC CH1 substitution at V68 is a V68L substitution. In some embodiments, the HC CH1 substitution at C103 is a C103S substitution. In some embodiments, the LC CL1 substitution at Q17 of SEQ ID NO: 6: is an Q17C substitution. In some embodiments, the LC CL1 substitution at E18 of SEQ ID NO: 1: is an E18C substitution. In some embodiments, the LC CL1 substitution at L28 of SEQ ID NO: 6 is a L28F substitution. In some embodiments, the LC CL1 substitution at L29 of SEQ ID NO: 1 is a L29F substitution. In some embodiments, the LC CL1 substitution at C107 of SEQ ID NO: 6 is a C107S substitution. In some embodiments, the LC CL1 substitution at C105 of SEQ ID NO: 1 is a C105S substitution. In some embodiments, the HC CH1 comprises or consists of an F9C, F53V, S661, V68L, and C103S substitution, and the LC CL comprises a Q17C, L28F, and a C107S substitution. In some embodiments, the HC CH1 comprises or consists of an F9C, F53V, S66I, V68L, and C103S substitution, and the LC CL comprises an E18C, L29F, and a C105S substitution at a position corresponding to F9, F53, S66, V68, and C103 of SEQ ID NO: 9 and E18, L29, and C105 of SEQ ID NO: 1. In some embodiments, the HC CH1 comprises or consists of an F9C, F53V, S66I, V68L, and C103S substitution, and the LC CL comprises a Q17C, L28F, and a C107S substitution at a position corresponding to F9, F53, S66, V68, and C103 of SEQ ID NO: 9 and Q17, L28, and C107 of SEQ ID NO: 6.

In some embodiments, the compositions disclosed herein comprise a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG, wherein the HC1 CH1 comprises or consists of a substitution at F126 and C220 (EU Numbering), the LC1 CL comprises or consists of a substitution at position 124 (E124 for lambda and Q124 for kappa), and C214 (EU Numbering), the HC2 CH1 comprises or consists of a substitution at F170, S183, and V185 (EU Numbering), and the LC2 CL comprises or consists of a substitution L135 (EU Numbering), wherein the HC1 CH1 and/or HC2 CH1 does not include any substitution selected from L128F, A141M, A141T, A141I, F170M, F170Y, F170S, F170A, S181I, S18IT, S181M, S183A, S183V, and V185A (EU Numbering), and wherein the LC1 CL and/or LC2 CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (EU Numbering). In some embodiments, the composition comprises a human IgG. In some embodiments, the composition comprises a human IgG1, IgG2, IgG3, or IgG4, antibody. In some embodiments, the composition comprises a chimeric antibody. In some embodiments, the composition comprises a humanized antibody. In some embodiments, the composition comprises a chimeric or humanized IgG1, IgG2, IgG3, or IgG4, antibody. In some embodiments, the HC1 CH1 substitution at F126 (EU Numbering) is an F126C substitution, and the LC1 CL substitution at position 124 (EU Numbering) is an E124C (lambda) or Q124C (kappa) substitution. In some embodiments, the LC CL1 substitution at position 124 (EU Numbering) is an E124C (lambda) substitution. In some embodiments, the LC CL1 substitution at position 124 (EU Numbering) is a Q124C (kappa) substitution. In some embodiments, the HC CH1 substitution at C220 (EU Numbering) is a C220S substitution. In some embodiments, the LC CL substitution at C214 (EU Numbering) is a C214S substitution.

Some embodiments herein are directed to an expression vector or construct for expression of one or more polynucleotides encoding a Fab and/or antibody comprising: a heavy chain (HC) variable domain (VH) 101, a heavy chain (HC) constant domain (CH1) of a human IgG 102, a light chain (LC) variable domain (VL) 103, and a light chain (LC) constant domain (CL) of a human IgG 104, wherein the HC CH1 comprises or consists of a substitution at F170 and S183, and optionally a substitution at V185 (EU Numbering), and the LC CL comprises or consists of a substitution at L135 (EU Numbering), wherein the HC1 does not include any substitution selected from L128F, A141M, A141T, A14II, F170M, F170Y, F170S, F170A, S181I, S181T, S181M, S183A, S183V, and V185A (EU Numbering), and wherein the CL does not include any substitution selected from F116A, F118V, S13IT, S131D, V133A, V133I, L135Y, L135V, S162A, S162M, T164S, S174A, S176M, S176A, S176T, S176F, T178L, T178V, and T178I (EU Numbering). In some embodiments, the CH1 consists of substitutions at F170 and S183 (EU Numbering), or at F170, S183 and V185 (EU Numbering), and wherein the CL consists of a substitution at L135 (EU Numbering). In some embodiments, V185 of CH1 is substituted. In some embodiments, the HC CH1 substitution at F170 (EU Numbering) is an F1701, or F170V substitution. In some embodiments, the expression vector or construct expresses a human antibody or fragment thereof. In some embodiments, the fragment comprises an antigen binding fragment. In some embodiments, expression vector or construct expresses a human IgG. In some embodiments, expression vector or construct expresses a human IgG1, IgG2, IgG3, or IgG4, antibody. In some embodiments, expression vector or construct expresses a chimeric antibody. In some embodiments, expression vector or construct expresses a humanized antibody. In some embodiments, expression vector or construct expresses a chimeric or humanized IgG1, IgG2, IgG3, or IgG4, antibody. In some embodiments, the HC CH1 substitution at S183 (EU Numbering) is a S183L or S183I substitution. In some embodiments, the HC CH1 substitution at V185 (EU Numbering) is a V185L substitution. In some embodiments, the LC CL1 substitution at L135 (EU Numbering) is a L135F substitution. In some embodiments, the HC CH1 comprises or consists of an F1701 and S183V (EU Numbering) substitution, and the LC CL comprises or consists of a L135F (EU Numbering) substitution. In some embodiments, the HC CH1 comprises or consists of an F170I, S183L, and V185L (EU Numbering) substitution, and the LC CL comprises a L135F (EU Numbering) substitution. In some embodiments, the HC CH1 comprises or consists of SEQ ID NO: 15, 19, 23, or 24, and the LC CL comprises or consists of SEQ ID NO: 2, 5, 7, or 8. In some embodiments, the HC CH1 comprises a substitution at F53 and S66, and V68 of SEQ ID NO: 9, and the LC CL comprises a substitution at L29 of SEQ ID NO: 1 or L28 of SEQ ID NO: 6. In some embodiments, the HC CH1 substitution at F53 of SEQ ID NO: 9 is an F53I, or F53V substitution. In some embodiments, the HC CH1 substitution at S66 of SEQ ID NO: 9 is a S66L or S66I substitution. In some embodiments, the HC CH1 substitution at V68 of SEQ ID NO: 9 is a V68L substitution. In some embodiments, the LC CL substitution at L29 of SEQ ID NO: 1 or L28 of SEQ ID NO: 6 is a L29F or L28F substitution.

Some embodiments herein are directed to an expression vector or construct for expression of one or more polynucleotides encoding a Fab and/or antibody comprising: a heavy chain (HC) variable domain (VH), a heavy chain (HC) constant domain (CH1) of a human IgG a light chain (LC) variable domain (VL), and a light chain (LC) constant domain (CL) of a human IgG, wherein the HC CH1 comprises or consists of a substitution at F126, F170, S185, V185, and C220 (EU Numbering), and the LC CL comprises or consists of a substitution at E124, L135, and C214 (lambda) or Q124, L135, and C214 (kappa) (EU Numbering) (in some embodiments the LC CL comprises or consists of a substitution at E124, L135, and C214 (lambda); in some embodiments the LC CL comprises or consists of a substitution at Q124, L135, and C214 (kappa)), wherein the HC1 does not include any substitution selected from L128F, A141M, A141T, A141I, F170M, F170Y, F170S, F170A, S181I, S181T, S181M, S183A, S183V, and V185A (EU Numbering), and wherein the CL does not include any substitution selected from F116A, F118V, S13IT, S131D, V133A, V133I, L13SY, L13SV, S162A, S162M, T164S, S174A, S176M, S176A, S176T, S176F, T178L, T178V, and T178I (EU Numbering). In some embodiments, the expression vector or construct expresses a polynucleotide encoding a human antibody or fragment thereof. In some embodiments, the fragment comprises an antigen binding fragment. In some embodiments, the expression vector or construct expresses a polynucleotide encoding a human IgG. In some embodiments, the expression vector or construct expresses a polynucleotide encoding a human IgG1, IgG2, IgG3, or IgG4, antibody. In some embodiments, the expression vector or construct expresses a polynucleotide encoding a chimeric antibody. In some embodiments, the expression vector or construct expresses a polynucleotide encoding a humanized antibody. In some embodiments, the expression vector or construct expresses a polynucleotide encoding a chimeric or humanized IgG1, IgG2, IgG3, or IgG4, antibody. In some embodiments, the CH1 comprises or consists of substitutions at F170 and S183 (EU Numbering), or at F170, S183 and V185 (EU Numbering), and wherein the CL comprises or consists of a substitution at L135 (EU Numbering). In some embodiments, the HC CH1 substitution at F126 (EU Numbering) is an F126C substitution. In some embodiments, the HC CH1 substitution at F170 (EU Numbering) is an F170V or F170I substitution. In some embodiments, the HC CH1 substitution at S183 (EU Numbering) is a S183L, or S183I substitution. In some embodiments, the HC CH1 substitution at V185 (EU Numbering) is a V185L substitution. In some embodiments, the HC CH1 substitution at C220 (EU Numbering) is a C220S substitution. In some embodiments, the LC CL1 substitution at position 124 (EU Numbering) is an E124C (lambda) or Q124C (kappa) substitution. In some embodiments, the LC CL1 substitution at position 124 (EU Numbering) is an E124C (lambda) substitution. In some embodiments, the LC CL1 substitution at position 124 (EU Numbering) is a Q124C (kappa) substitution. In some embodiments, the LC CL1 substitution at L135 (EU Numbering) is a L135F substitution. In some embodiments, the LC CL1 substitution at C214 (EU Numbering) is a C214S substitution. In some embodiments, the HC CH1 comprises or consists of an F126C, F170V, S1831, V185L, and C220S (EU Numbering) substitution, and the LC CL comprises or consists of an E124C, L135F, and C214S (lambda) or Q124C, L135F, and C214S (kappa) (EU Numbering) substitution. In some embodiments, the HC CH1 comprises or consists of an F126C, F170I, S183L, V185L, and C220S (EU Numbering) substitution, and the LC CL comprises an E124C, L135F, and C214S (lambda) or Q124C, L135F, and C214S (kappa) (BU Numbering) substitution. In some embodiments, the LC CL comprises an E124C, L135F, and C214S (lambda) (EU Numbering) substitution. In some embodiments, the LC CL comprises a Q124C, L135F, and C214S (kappa) (EU Numbering) substitution. In some embodiments, the HC CH1 comprises or consists of SEQ ID NO: 15, 19, 23, or 24, and the LC CL comprises or consists of SEQ ID NO: 2, 5, 7, or 8. In some embodiments, the CH1 consists of substitutions at F9, F53, S66, V68, and C103, of SEQ ID NO: 9 and wherein the LC CL consists of a substitution at E18, L29, and C105 of SEQ ID NO: 1. In some embodiments, the CH1 consists of substitutions at F9, F53, S66, V68, and C103, of SEQ ID NO: 9, and wherein the LC CL consists of a substitution at Q17, L28, and C107 of SEQ ID NO: 6. In some embodiments, the HC CH1 substitution at F9 is an F9C substitution. In some embodiments, the HC CH1 substitution at F53 is an F53V or F53I substitution. In some embodiments, the HC CH1 substitution at S66 is a S66L, or S66I substitution. In some embodiments, the HC CH1 substitution at V68 is a V68L substitution. In some embodiments, the HC CH1 substitution at C103 is a C103S substitution. In some embodiments, the LC CL1 substitution at Q17 of SEQ ID NO: 6: is an Q17C substitution. In some embodiments, the LC CL1 substitution at E18 of SEQ ID NO: 1: is an E18C substitution. In some embodiments, the LC CL1 substitution at L28 of SEQ ID NO: 6 is a L28F substitution. In some embodiments, the LC CL1 substitution at L29 of SEQ ID NO: 1 is a L29F substitution. In some embodiments, the LC CL1 substitution at C107 of SEQ ID NO: 6 is a C107S substitution. In some embodiments, the LC CL1 substitution at C105 of SEQ ID NO: 1 is a C105S substitution. In some embodiments, the HC CH1 comprises or consists of an F9C, F53V, S66I, V68L, and C103S substitution, and the LC CL comprises a Q17C, L28F, and a C107S substitution. In some embodiments, the HC CH1 comprises or consists of an F9C, F53V, S66I, V68L, and C103S substitution, and the LC CL comprises an E18C, L29F, and a C105S substitution at a position corresponding to F9, F53, S66, V68, and C103 of SEQ ID NO: 9 and E18, L29, and C105 of SEQ ID NO: 1. In some embodiments, the HC CH1 comprises or consists of an F9C, F53V, S66I, V68L, and C103S substitution, and the LC CL comprises a Q17C, L28F, and a C107S substitution at a position corresponding to F9, F53, S66, V68, and C103 of SEQ ID NO: 9 and Q17, L28, and C107 of SEQ ID NO: 6.

Some embodiments herein are directed to an expression vector or construct for expression of one or more polynucleotides encoding a Fab and/or antibody comprising: a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG, wherein the HC1 CH1 comprises or consists of a substitution at F126 and C220 (EU Numbering), the LC1 CL comprises or consists of a substitution at position 124 (E124 for lambda and Q124 for kappa), and C214 (EU Numbering), the HC2 CH1 comprises or consists of a substitution at F170, S183, and V185 (EU Numbering), and the LC2 CL comprises or consists of a substitution L135 (EU Numbering), wherein the HC1 CH1 and/or HC2 CH1 does not include any substitution selected from L128F, A141M, A141T, A141I, F170M, F170Y, F170S, F170A, S181I, S181T, S181M, S183A, S183V, and V185A (EU Numbering), and wherein the LC1 CL and/or LC2 CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (EU Numbering). In some embodiments, the expression vector or construct expresses a polynucleotide encoding a human antibody or fragment thereof. In some embodiments, the fragment comprises an antigen binding fragment. In some embodiments, the expression vector or construct expresses a polynucleotide encoding a human IgG. In some embodiments, the expression vector or construct expresses a polynucleotide encoding a human IgG1, IgG2, IgG3, or IgG4, antibody. In some embodiments, the expression vector or construct expresses a polynucleotide encoding a chimeric antibody. In some embodiments, the expression vector or construct expresses a polynucleotide encoding a humanized antibody. In some embodiments, the expression vector or construct expresses a polynucleotide encoding a chimeric or humanized IgG1, IgG2, IgG3, or IgG4, antibody. In some embodiments, the HC1 CH1 substitution at F126 (EU Numbering) is an F126C substitution, and the LC1 CL substitution at position 124 (EU Numbering) is an E124C (lambda) or Q124C (kappa) substitution. In some embodiments, the LC CL1 substitution at position 124 (EU Numbering) is an E124C (lambda) substitution. In some embodiments, the LC CL1 substitution at position 124 (EU Numbering) is a Q124C (kappa) substitution. In some embodiments, the HC CH1 substitution at C220 (EU Numbering) is a C220S substitution. In some embodiments, the LC CL substitution at C214 (EU Numbering) is a C214S substitution.

Some embodiments herein are directed to a cell, comprising one or more polynucleotides, expression vectors, or constructs, encoding a Fab and/or antibody comprising: a heavy chain (HC) variable domain (VH) 101, a heavy chain (HC) constant domain (CH1) of a human IgG 102, a light chain (LC) variable domain (VL) 103, and a light chain (LC) constant domain (CL) of a human IgG 104, wherein the HC CH1 comprises or consists of a substitution at F170 and S183, and optionally a substitution at V185 (EU Numbering), and the LC CL comprises or consists of a substitution at L135 (EU Numbering), wherein the HC1 does not include any substitution selected from L128F, A141M, A141T, A141I, F170M, F170Y, F170S, F170A, S181I, S181T, S181M, S183A, S183V, and V185A (EU Numbering), and wherein the CL does not include any substitution selected from F116A, F118V, S131T, S131D, V133A, V133I, L135Y, L135V, S162A, S162M, T164S, S174A, S176M, S176A, S176T, S176F, T178L, T178V, and T1781 (EU Numbering). In some embodiments, the CH1 consists of substitutions at F170 and S183 (EU Numbering), or at F170, S183 and V185 (EU Numbering), and wherein the CL consists of a substitution at L135 (EU Numbering). In some embodiments, the HC CH1 substitution at F170 (EU Numbering) is an F1701, or F170V substitution. In some embodiments, the cell comprises one or more polynucleotides, expression vectors, or constructs, encoding a human antibody or fragment thereof. In some embodiments, the fragment comprises an antigen binding fragment. In some embodiments, the cell comprises one or more polynucleotides, expression vectors, or constructs, encoding a human IgG. In some embodiments, the cell comprises one or more polynucleotides, expression vectors, or constructs, encoding a human IgG1, IgG2, IgG3, or IgG4, antibody. In some embodiments, the cell comprises one or more polynucleotides, expression vectors, or constructs, encoding a chimeric antibody. In some embodiments, the cell comprises one or more polynucleotides, expression vectors, or constructs, encoding a polynucleotide encoding a humanized antibody. In some embodiments, the cell comprises one or more polynucleotides, expression vectors, or constructs, encoding a chimeric or humanized IgG1, IgG2, IgG3, or IgG4, antibody. In some embodiments, the HC CH1 substitution at S183 (EU Numbering) is a S183L or S183I substitution. In some embodiments, the HC CH1 substitution at V185 (EU Numbering) is a V185L substitution. In some embodiments, the LC CL1 substitution at L135 (EU Numbering) is a L135F substitution. In some embodiments, the HC CH1 comprises or consists of an F1701 and S183V (EU Numbering) substitution, and the LC CL comprises or consists of a L135F (EU Numbering) substitution. In some embodiments, the HC CH1 comprises or consists of an F1701, S183L, and V185L (EU Numbering) substitution, and the LC CL comprises or consists of a L135F (EU Numbering) substitution. In some embodiments, the HC CH1 comprises or consists of SEQ ID NO: 15, 19, 23, or 24, and the LC CL comprises or consists of SEQ ID NO: 2, 5, 7, or 8. In some embodiments, the HC CH1 comprises a substitution at F53 and S66, and V68 of SEQ ID NO: 9, and the LC CL comprises a substitution at L29 of SEQ ID NO: 1 or L28 of SEQ ID NO: 6. In some embodiments, the HC CH1 substitution at F53 of SEQ ID NO: 9 is an F53I, or F53V substitution. In some embodiments, the HC CH1 substitution at S66 of SEQ ID NO: 9 is a S66L or S661 substitution. In some embodiments, the HC CH1 substitution at V68 of SEQ ID NO: 9 is a V68L substitution. In some embodiments, the LC CL substitution at L29 of SEQ ID NO: 1 or L28 of SEQ ID NO: 6 is a L29F or L28F substitution. In some embodiments, the cell is a mammalian cell or bacterial cell. In some embodiments, the cell is a HEK293 cell, Chinese hamster ovary (CHO) cell, or a NS0 cell.

Some embodiments herein are directed to a cell, comprising one or more polynucleotides, expression vectors, or constructs, encoding a Fab and/or antibody comprising: a heavy chain (HC) variable domain (VH), a heavy chain (HC) constant domain (CH1) of a human IgG a light chain (LC) variable domain (VL), and a light chain (LC) constant domain (CL) of a human IgG, wherein the HC CH1 comprises or consists of a substitution at F126, F176, S189, V191, and C220 (EU Numbering), and the LC CL comprises or consists of a substitution at E124, L135, and C214 (lambda) or Q124, L135, and C214 (kappa) (EU Numbering), wherein the HC1 does not include any substitution selected from L128F, A141M, A141T, A141I, F170M, F170Y, F170S, F170A, S181I, S181T, S181M, S183A, S183V, and V185A (EU Numbering), and wherein the CL does not include any substitution selected from F116A, F118V, S13IT, S131D, V133A, V133I, L135Y, L135V, S162A, S162M, T164S, S174A, S176M, S176A, S176T, S176F, T178L, T178V, and T178I (EU Numbering). In some embodiments, the CH1 consists of substitutions at F170 and S183 (EU Numbering), or at F170, S183 and V185 (EU Numbering), and wherein the CL consists of a substitution at L135 (EU Numbering). In some embodiments, the HC CH1 comprises a substitution at F53 and S66, and V68 of SEQ ID NO: 9, and the LC CL comprises a substitution at L29 of SEQ ID NO: 1 or L28 of SEQ ID NO: 6. In some embodiments, the HC CH1 substitution at F53 of SEQ ID NO: 9 is an F53I, or F53V substitution. In some embodiments, the HC CH1 substitution at S66 of SEQ ID NO: 9 is a S66L or S66I substitution. In some embodiments, the HC CH1 substitution at V68 of SEQ ID NO: 9 is a V68L substitution. In some embodiments, the LC CL substitution at L29 of SEQ ID NO: 1 or L28 of SEQ ID NO: 6 is a L29F or L28F substitution. In some embodiments, the cell comprises one or more polynucleotides, expression vectors, or constructs, encoding a human antibody or fragment thereof. In some embodiments, the fragment comprises an antigen binding fragment. In some embodiments, the cell comprises one or more polynucleotides, expression vectors, or constructs, encoding a human IgG. In some embodiments, the cell comprises one or more polynucleotides, expression vectors, or constructs, encoding a human IgG1, IgG2, IgG3, or IgG4, antibody. In some embodiments, the cell comprises one or more polynucleotides, expression vectors, or constructs, encoding a chimeric antibody. In some embodiments, the cell comprises one or more polynucleotides, expression vectors, or constructs, encoding a polynucleotide encoding a humanized antibody. In some embodiments, the HC CH1 substitution at F126 (EU Numbering) is an F126C substitution. In some embodiments, the HC CH1 substitution at F170 (EU Numbering) is an F170V or F1701 substitution. In some embodiments, the HC CH1 substitution at S183 (EU Numbering) is a S183L, or S183I substitution. In some embodiments, the HC CH1 substitution at V185 (EU Numbering) is a V185L substitution. In some embodiments, the HC CH1 substitution at C220 (EU Numbering) is a C220S substitution. In some embodiments, the LC CL1 substitution at position 124 (EU Numbering) is an E124C (lambda) or Q124C (kappa) substitution. In some embodiments, the LC CL1 substitution at position 124 (EU Numbering) is an E124C (lambda) substitution. In some embodiments, the LC CL1 substitution at position 124 (EU Numbering) is a Q124C (kappa) substitution. In some embodiments, the LC CL1 substitution at L135 (EU Numbering) is a L135F substitution. In some embodiments, the LC CL1 substitution at C214 (EU Numbering) is a C214S substitution. In some embodiments, the HC CH1 comprises or consists of an F126C, F170V, S183I, V185L, and C220S (EU Numbering) substitution, and the LC CL comprises or consists of an E124C, L135F, and C214S (lambda) or Q124C, L135F, and C214S (kappa) (EU Numbering) substitution. In some embodiments, the LC CL comprises or consists of an E124C, L135F, and C214S (lambda) (EU Numbering) substitution. In some embodiments, the LC CL comprises or consists of a Q124C, L135F, and C214S (kappa) (EU Numbering) substitution. In some embodiments, the HC CH1 comprises or consists of an F126C, F170I, S183L, V185L, and C220S (EU Numbering) substitution, and the LC CL comprises or consists of an E124C, L135F, and C214S (lambda) or Q124C, L135F, and C214S (kappa) (EU Numbering) substitution. In some embodiments, the LC CL comprises or consists of an E124C, L135F, and C214S (lambda) (EU Numbering) substitution. In some embodiments, the LC CL comprises or consists of a Q124C, L135F, and C214S (kappa) (EU Numbering) substitution. In some embodiments, the HC CH1 comprises or consists of SEQ ID NO: 15, 19, 23 or 24, and the LC CL comprises or consists of SEQ ID NO: 2, 5, 7, or 8. In some embodiments, the CH1 consists of substitutions at F9, F53, S66, V68, and C103, of SEQ ID NO: 9 and wherein the LC CL consists of a substitution at E18, L29, and C105 of SEQ ID NO: 1. In some embodiments, the CH1 consists of substitutions at F9, F53, S66, V68, and C103 of SEQ ID NO: 9, and wherein the LC CL consists of a substitution at Q17, L28, and C107 of SEQ ID NO: 6. In some embodiments, the HC CH1 substitution at F9 is an F9C substitution. In some embodiments, the HC CH1 substitution at F53 is an F53V or F53I substitution. In some embodiments, the HC CH1 substitution at S66 is a S66L, or S66I substitution. In some embodiments, the HC CH1 substitution at V68 is a V68L substitution. In some embodiments, the HC CH1 substitution at C103 is a C103S substitution. In some embodiments, the LC CL1 substitution at Q17 of SEQ ID NO: 6: is an Q17C substitution. In some embodiments, the LC CL1 substitution at E18 of SEQ ID NO: 1: is an E18C substitution. In some embodiments, the LC CL1 substitution at L28 of SEQ ID NO: 6 is a L28F substitution. In some embodiments, the LC CL1 substitution at L29 of SEQ ID NO: 1 is a L29F substitution. In some embodiments, the LC CL1 substitution at C107 of SEQ ID NO: 6 is a C107S substitution. In some embodiments, the LC CL1 substitution at C105 of SEQ ID NO: 1 is a C105S substitution. In some embodiments, the HC CH1 comprises or consists of an F9C, F53V, S66I, V68L, and C103S substitution, and the LC CL comprises a Q17C, L28F, and a C107S (EU Numbering) substitution. In some embodiments, the HC CH1 comprises or consists of an F9C, F53V, S66I, V68L, and C103S substitution, and the LC CL comprises an E18C, L29F, and a C105S substitution at a position corresponding to F9, F53, S66, V68, and C103 of SEQ ID NO: 9 and E18, L29, and C105 of SEQ ID NO: 1. In some embodiments, the HC CH1 comprises or consists of an F9C, F53V, S66I, V68L, and C103S substitution, and the LC CL comprises a Q17C, L28F, and a C107S substitution at a position corresponding to F9, F53, 866, V68, and C103 of SEQ ID NO: 9 and Q17, L28, and C107 of SEQ ID NO: 6. In some embodiments, the cell is a mammalian cell or bacterial cell. In some embodiments, the cell is a HEK293 cell, Chinese hamster ovary (CHO) cell, or a NS0 cell.

Some embodiments herein are directed to a cell, comprising one or more polynucleotides, expression vectors, or constructs, encoding a Fab and/or antibody comprising: a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG, wherein the HC1 CH1 comprises or consists of a substitution at F126 and C220 (EU Numbering), the LC1 CL comprises or consists of a substitution at position 124 (E124 for lambda and Q124 for kappa), and C214 (EU Numbering), the HC2 CH1 comprises or consists of a substitution at F170, S183, and V185 (EU Numbering), and the LC2 CL comprises or consists of a substitution L135 (EU Numbering), wherein the HC1 CH1 and/or HC2 CH1 does not include any substitution selected from L128F, A141M, A141T, A141I, F170M, F170Y, F170S, F170A, S181I, S181T, S181M, S183A, S183V, and V185A (EU Numbering), and wherein the LC1 CL and/or LC2 CL does not include any substitution selected from F116A, F118V, S131T, S131D, V133A, V133I, L135Y, L135V, S162A, S162M, T164S, S174A, S176M, S176A, S176T, S176F, T178L, T178V, and T1781 (EU Numbering). In some embodiments, the cell comprises one or more polynucleotides, expression vectors, or constructs, encoding a human antibody or fragment thereof. In some embodiments, the fragment comprises an antigen binding fragment. In some embodiments, the cell comprises one or more polynucleotides, expression vectors, or constructs, encoding a human IgG. In some embodiments, the cell comprises one or more polynucleotides, expression vectors, or constructs, encoding a human IgG1, IgG2, IgG3, or IgG4, antibody. In some embodiments, the cell comprises one or more polynucleotides, expression vectors, or constructs, encoding a chimeric antibody. In some embodiments, the cell comprises one or more polynucleotides, expression vectors, or constructs, encoding a polynucleotide encoding a humanized antibody. In some embodiments, the HC1 CH1 substitution at F126 (EU Numbering) is an F126C substitution, and the LC1 CL substitution at position 124 (EU Numbering) is an E124C (lambda) or Q124C (kappa) substitution. In some embodiments, the LC CL1 substitution at position 124 (EU Numbering) is an E124C (lambda) substitution. In some embodiments, the LC CL1 substitution at position 124 (EU Numbering) is a Q124C (kappa) substitution. In some embodiments, the HC CH1 substitution at C220 (EU Numbering) is a C220S substitution. In some embodiments, the LC CL substitution at C214 (EU Numbering) is a C214S substitution. In some embodiments, the CH1 consists of substitutions at F9, F53, S66, V68, and C103 of SEQ ID NO: 9 and wherein the LC CL consists of a substitution at E18, L29, and C105 of SEQ ID NO: 1. In some embodiments, the CH1 consists of substitutions at F9, F53, S66, V68, and C103 of SEQ ID NO: 9, and wherein the LC CL consists of a substitution at Q17, L28, and C107 of SEQ ID NO: 6. In some embodiments, the HC CH1 substitution at F9 is an F9C substitution. In some embodiments, the HC CH1 substitution at F53 is an F53V or F53I substitution. In some embodiments, the HC CH1 substitution at S66 is a S66L, or S66I substitution. In some embodiments, the HC CH1 substitution at V68 is a V68L substitution. In some embodiments, the HC CH1 substitution at C103 is a C103S substitution. In some embodiments, the LC CL1 substitution at Q17 of SEQ ID NO: 6: is an Q17C substitution. In some embodiments, the LC CL1 substitution at E18 of SEQ ID NO: 1: is an E18C substitution. In some embodiments, the LC CL1 substitution at L28 of SEQ ID NO: 6 is a L28F substitution.

In some embodiments, the LC CL1 substitution at L29 of SEQ ID NO: 1 is a L29F substitution. In some embodiments, the LC CL1 substitution at C107 of SEQ ID NO: 6 is a C107S substitution. In some embodiments, the LC CL1 substitution at C105 of SEQ ID NO: 1 is a C105S substitution. In some embodiments, the HC CH1 comprises or consists of an F9C, F53V, S661, V68L, and C103S substitution, and the LC CL comprises a Q17C, L28F, and a C107S (EU Numbering) substitution. In some embodiments, the HC CH1 comprises or consists of an F9C, F53V, S66I, V68L, and C103S substitution, and the LC CL comprises an E18C, L29F, and a C105S substitution at a position corresponding to F9, F53, S66, V68, and C103 of SEQ ID NO: 9 and E18, L29, and C105 of SEQ ID NO: 1. In some embodiments, the HC CH1 comprises or consists of an F9C, F53V, S66I, V68L, and C103S substitution, and the LC CL comprises a Q17C, L28F, and a C107S substitution at a position corresponding to F9, F53, S66, V68, and C103 of SEQ ID NO: 9 and Q17, L28, and C107 of SEQ ID NO: 6.

Some embodiments herein are directed to a pharmaceutical composition comprising a Fab and/or antibody comprising one or more polynucleotides, expression vectors, or constructs, encoding a Fab and/or antibody comprising: a heavy chain (HC) variable domain (VH) 101, a heavy chain (HC) constant domain (CH1) of a human IgG 102, a light chain (LC) variable domain (VL) 103, and a light chain (LC) constant domain (CL) of a human IgG 104, wherein the HC CH1 comprises or consists of a substitution at F170 and S183, and optionally a substitution at V185 (EU Numbering), and the LC CL comprises or consists of a substitution at L135 (EU Numbering), wherein the HC1 does not include any substitution selected from L128F, A141M, A14IT, A141I, F170M, F170Y, F170S, F170A, S181I, S181T, S181M, S183A, S183V, and V185A (EU Numbering), and wherein the CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (EU Numbering). In some embodiments, the pharmaceutical composition comprises a human antibody or fragment thereof. In some embodiments, the fragment comprises an antigen binding fragment. In some embodiments, the pharmaceutical composition comprises a human IgG. In some embodiments, the pharmaceutical composition comprises a human IgG1, IgG2, IgG3, or IgG4, antibody. In some embodiments, the pharmaceutical composition comprises a chimeric antibody. In some embodiments, the pharmaceutical composition comprises a polynucleotide encoding a humanized antibody. In some embodiments, the CH1 consists of substitutions at F170 and S183 (EU Numbering), or at F170, S183 and V185 (EU Numbering), and wherein the CL consists of a substitution at L135 (EU Numbering). In some embodiments, V185 of CH1 is substituted. In some embodiments, the HC CH1 substitution at F170 (EU Numbering) is an F170I, or F170V substitution. In some embodiments, the HC CH1 substitution at S183 (EU Numbering) is a S183L or S1831 substitution. In some embodiments, the HC CH1 substitution at V185 (EU Numbering) is a V185L substitution. In some embodiments, the LC CL1 substitution at L135 (EU Numbering) is a L135F substitution. In some embodiments, the HC CH1 comprises or consists of an F1701 and S183V (EU Numbering) substitution, and the LC CL comprises or consists of a L135F (EU Numbering) substitution. In some embodiments, the HC CH1 comprises or consists of an F170I, S183L, and V18SL (EU Numbering) substitution, and the LC CL comprises or consists of a L135F (EU Numbering) substitution. In some embodiments, the HC CH1 comprises or consists of SEQ ID NO: 15, 19, 23, or 24, and the LC CL comprises or consists of SEQ ID NO: 2, 5, 7, or 8. In some embodiments, the HC CH1 comprises a substitution at F53 and S66, and V68 of SEQ ID NO: 9, and the LC CL comprises a substitution at L29 of SEQ ID NO: 1 or L28 of SEQ ID NO: 6. In some embodiments, the HC CH1 substitution at F53 of SEQ ID NO: 9 is an F53I, or F53V substitution. In some embodiments, the HC CH1 substitution at S66 of SEQ ID NO: 9 is a S66L or S66I substitution. In some embodiments, the HC CH1 substitution at V68 of SEQ ID NO: 9 is a V68L substitution. In some embodiments, the LC CL substitution at L29 of SEQ ID NO: 1 or L28 of SEQ ID NO: 6 is a L29F or L28F substitution. In some embodiments, the pharmaceutical composition is a cell culture.

Some embodiments herein are directed to a pharmaceutical composition comprising a Fab and/or antibody comprising one or more polynucleotides, expression vectors, or constructs, encoding a Fab and/or antibody comprising: a heavy chain (HC) variable domain (VH), a heavy chain (HC) constant domain (CH1) of a human IgG a light chain (LC) variable domain (VL), and a light chain (LC) constant domain (CL) of a human IgG, wherein the HC CH1 comprises or consists of a substitution at F126, F176, S189, V191, and C220 (EU Numbering), and the LC CL comprises or consists of a substitution at E124, L135, and C214 (lambda) or Q124, L135, and C214 (kappa) (EU Numbering) (in some embodiments the LC CL comprises or consists of a substitution at E124, L135, and C214 (lambda); in some embodiments the LC CL comprises or consists of a substitution at Q124, L135, and C214 (kappa)), wherein the HC1 does not include any substitution selected from L128F, A141M, A141T, A141I, F170M, F170Y, F170S, F170A, S181I, S181T, S181M, S183A, S183V, and V18SA (EU Numbering), and wherein the CL does not include any substitution selected from F116A, F118V, S131T, S131D, V133A, V133I, L135Y, L135V, S162A, S162M, T164S, S174A, S176M, S176A, S176T, S176F, T178L, T178V, and T1781 (EU Numbering). In some embodiments, the pharmaceutical composition comprises a human antibody or fragment thereof. In some embodiments, the fragment comprises an antigen binding fragment. In some embodiments, the pharmaceutical composition comprises a human IgG. In some embodiments, the pharmaceutical composition comprises a human IgG1, IgG2, IgG3, or IgG4, antibody. In some embodiments, the pharmaceutical composition comprises a chimeric antibody. In some embodiments, the pharmaceutical composition comprises a polynucleotide encoding a humanized antibody. In some embodiments, the CH1 consists of substitutions at F170 and S183 (EU Numbering), or at F170, S183 and V185 (EU Numbering), and wherein the CL consists of a substitution at L135 (EU Numbering). In some embodiments, the HC CH1 substitution at F126 (EU Numbering) is an F126C substitution. In some embodiments, the HC CH1 substitution at F170 (EU Numbering) is an F170V or F1701 substitution. In some embodiments, the HC CH1 substitution at S189 (EU Numbering) is a S183L, or S183I substitution. In some embodiments, the HC CH1 substitution at V185 (EU Numbering) is a V185L substitution. In some embodiments, the HC CH1 substitution at C220 (EU Numbering) is a C220S substitution. In some embodiments, the LC CL1 substitution at position 124 (EU Numbering) is an E124C (lambda) or Q124C (kappa) substitution. In some embodiments, the LC CL1 substitution at position 124 (EU Numbering) is an E124C (lambda) substitution. In some embodiments, the LC CL1 substitution at position 124 (EU Numbering) is a Q124C (kappa) substitution. In some embodiments, the LC CL1 substitution at L135 (EU Numbering) is a L135F substitution. In some embodiments, the LC CL1 substitution at C214 (EU Numbering) is a C214S substitution. In some embodiments, the HC CH1 comprises or consists of an F126C, F170V, S183I, V185L, and C220S (EU Numbering) substitution, and the LC CL comprises or consists of an E124C, L135F, and C214S (lambda) or Q124C, L135F, and C214S (kappa) (EU Numbering) substitution. In some embodiments, the LC CL comprises or consists of an E124C, L135F, and C214S (lambda) (EU Numbering) substitution. In some embodiments, the LC CL comprises or consists of a Q124C, L135F, and C214S (kappa) (EU Numbering) substitution. In some embodiments, the HC CH1 comprises or consists of an F126C, F170I, S183L, V185L, and C220S (EU Numbering) substitution, and the LC CL comprises or consists of an E124C, L135F, and C214S (lambda) or Q124C, L135F, and C214S (kappa) (EU Numbering) substitution. In some embodiments, the LC CL comprises or consists of an E124C, L135F, and C214S (lambda) (EU Numbering) substitution. In some embodiments, the LC CL comprises or consists of a Q124C, L135F, and C214S (kappa) (EU Numbering) substitution. In some embodiments, the HC CH1 comprises or consists of SEQ ID NO: 15, 19, 23, or 24, and the LC CL comprises or consists of SEQ ID NO: 2, 5, 7, or 8. In some embodiments, the CH1 consists of substitutions at F9, F53, S66, V68, and C103, of SEQ ID NO: 9 and wherein the LC CL consists of a substitution at E18, L29, and C105 of SEQ ID NO: 1. In some embodiments, the CH1 consists of substitutions at F9, F53, S66, V68, and C103, of SEQ ID NO: 9, and wherein the LC CL consists of a substitution at Q17, L28, and C107 of SEQ ID NO: 6. In some embodiments, the HC CH1 substitution at F9 is an F9C substitution. In some embodiments, the HC CH1 substitution at F53 is an F53V or F531 substitution. In some embodiments, the HC CH1 substitution at S66 is a S66L, or S66I substitution. In some embodiments, the HC CH1 substitution at V68 is a V68L substitution. In some embodiments, the HC CH1 substitution at C103 is a C103S substitution. In some embodiments, the LC CL1 substitution at Q17 of SEQ ID NO: 6: is an Q17C substitution. In some embodiments, the LC CL1 substitution at E18 of SEQ ID NO: 1: is an E18C substitution. In some embodiments, the LC CL1 substitution at L28 of SEQ ID NO: 6 is a L28F substitution. In some embodiments, the LC CL1 substitution at L29 of SEQ ID NO: 1 is a L29F substitution. In some embodiments, the LC CL1 substitution at C107 of SEQ ID NO: 6 is a C107S substitution. In some embodiments, the LC CL1 substitution at C105 of SEQ ID NO: 1 is a C105S substitution. In some embodiments, the HC CH1 comprises or consists of an F9C, F53V, S66I, V68L, and C103S substitution, and the LC CL comprises a Q17C, L28F, and a C107S (EU Numbering) substitution. In some embodiments, the HC CH1 comprises or consists of an F9C, F53V, S66I, V68L, and C103S substitution, and the LC CL comprises an E18C, L29F, and a C105S substitution at a position corresponding to F9, F53, S66, V68, and C103 of SEQ ID NO: 9 and E18, L29, and C105 of SEQ ID NO: 1. In some embodiments, the HC CH1 comprises or consists of an F9C, F53V, S66I, V68L, and C103S substitution, and the LC CL comprises a Q17C, L28F, and a C107S substitution at a position corresponding to F9, F53, S66, V68, and C103 of SEQ ID NO: 9 and Q17, 128, and C107 of SEQ ID NO: 6. In some embodiments, the pharmaceutical composition is a cell culture.

In some embodiments, the compositions disclosed herein comprises a cell supernatant and/or cell lysate of a cell comprising one or more polynucleotides, expression vectors, or constructs, encoding a Fab and/or antibody comprising: a heavy chain (HC) variable domain (VH) 101, a heavy chain (HC) constant domain (CH1) of a human IgG 102, a light chain (LC) variable domain (VL) 103, and a light chain (LC) constant domain (CL) of a human IgG 104, wherein the HC CH1 comprises or consists of a substitution at F170 and S183, and optionally a substitution at V185 (EU Numbering), and the LC CL comprises or consists of a substitution at L135 (EU Numbering), wherein the HC1 does not include any substitution selected from L128F, A141M, A141T, A141I, F170M, F170Y, F170S, F170A, S181I, S18IT, S181M, S183A, S183V, and V185A (EU Numbering), and wherein the CL does not include any substitution selected from F116A, F118V, S13IT, S131D, V133A, V133I, L135Y, L135V, S162A, S162M, T164S, S174A, S176M, S176A, S176T, S176F, T178L, T178V, and T178I (EU Numbering). In some embodiments, the cell comprises one or more polynucleotides, expression vectors, or constructs, encoding a human antibody or fragment thereof. In some embodiments, the fragment comprises an antigen binding fragment. In some embodiments, the cell comprises one or more polynucleotides, expression vectors, or constructs, encoding a human IgG. In some embodiments, the cell comprises one or more polynucleotides, expression vectors, or constructs, encoding a human IgG1, IgG2, IgG3, or IgG4, antibody. In some embodiments, the cell comprises one or more polynucleotides, expression vectors, or constructs, encoding a chimeric antibody. In some embodiments, the cell comprises one or more polynucleotides, expression vectors, or constructs, encoding a polynucleotide encoding a humanized antibody. In some embodiments, the CH1 consists of substitutions at F170 and S183 (EU Numbering), or at F170, S183 and V185 (EU Numbering), and wherein the CL consists of a substitution at L135 (EU Numbering). In some embodiments, V185 of CH1 is substituted. In some embodiments, the HC CH1 substitution at F170 (EU Numbering) is an F170I, or F170V substitution. In some embodiments, the HC CH1 substitution at S183 (EU Numbering) is a S183L or S183I substitution. In some embodiments, the HC CH1 substitution at V185 (EU Numbering) is a V185L substitution. In some embodiments, the LC CL1 substitution at L135 (EU Numbering) is a L135F substitution. In some embodiments, the HC CH1 comprises or consists of an F1701 and S183V (EU Numbering) substitution, and the LC CL comprises or consists of a L135F (EU Numbering) substitution. In some embodiments, the HC CH1 comprises or consists of an F170I, S183L, and V185L (EU Numbering) substitution, and the LC CL comprises or consists of a L135F (EU Numbering) substitution. In some embodiments, the HC CH1 comprises or consists of SEQ ID NO: 15, 19, 23, or 24, and the LC CL comprises or consists of SEQ ID NO: 2, 5, 7, or 8. In some embodiments, the HC CH1 comprises a substitution at F53 and S66, and V68 of SEQ ID NO: 9, and the LC CL comprises a substitution at L29 of SEQ ID NO: 1 or L28 of SEQ ID NO: 6. In some embodiments, the HC CH1 substitution at F53 of SEQ ID NO: 9 is an F53I, or F53V substitution. In some embodiments, the HC CH1 substitution at S66 of SEQ ID NO: 9 is a S66L or S66I substitution. In some embodiments, the HC CH1 substitution at V68 of SEQ ID NO: 9 is a V68L substitution. In some embodiments, the LC CL substitution at L29 of SEQ ID NO: 1 or L28 of SEQ ID NO: 6 is a L29F or L28F substitution. In some embodiments, the pharmaceutical composition is a cell culture.

In some embodiments, the compositions disclosed herein comprises a cell supernatant and/or cell lysate of a cell comprising one or more polynucleotides, expression vectors, or constructs, encoding a Fab and/or antibody comprising: a heavy chain (HC) variable domain (VH), a heavy chain (HC) constant domain (CH1) of a human IgG a light chain (LC) variable domain (VL), and a light chain (LC) constant domain (CL) of a human IgG, wherein the HC CH1 comprises or consists of a substitution at F126, F176, S189, V191, and C220 (EU Numbering), and the LC CL comprises or consists of a substitution at E124, L135, and C214 (lambda) or Q124, L135, and C214 (kappa) (EU Numbering) (in some embodiments the LC CL comprises or consists of a substitution at E124, L135, and C214 (lambda); in some embodiments the LC CL comprises or consists of a substitution at Q124, L135, and C214 (kappa)), wherein the HC1 does not include any substitution selected from L128F, A141M, A141T, A141I, F170M, F170Y, F170S, F170A, S181I, S181T, S181M, S183A, S183V, and V185A (EU Numbering), and wherein the CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (EU Numbering). In some embodiments, the cell comprises one or more polynucleotides, expression vectors, or constructs, encoding a human antibody or fragment thereof. In some embodiments, the fragment comprises an antigen binding fragment. In some embodiments, the cell comprises one or more polynucleotides, expression vectors, or constructs, encoding a human IgG. In some embodiments, the cell comprises one or more polynucleotides, expression vectors, or constructs, encoding a human IgG1, IgG2, IgG3, or IgG4, antibody. In some embodiments, the cell comprises one or more polynucleotides, expression vectors, or constructs, encoding a chimeric antibody. In some embodiments, the cell comprises one or more polynucleotides, expression vectors, or constructs, encoding a polynucleotide encoding a humanized antibody. In some embodiments, the CH1 consists of substitutions at F170 and S183 (EU Numbering), or at F170, S183 and V185 (EU Numbering), and wherein the CL consists of a substitution at L135 (EU Numbering). In some embodiments, the HC CH1 substitution at F126 (EU Numbering) is an F126C substitution. In some embodiments, the HC CH1 substitution at F170 (EU Numbering) is an F170V or F170I substitution. In some embodiments, the HC CH1 substitution at S183 (EU Numbering) is a S183L, or S183I substitution. In some embodiments, the HC CH1 substitution at V185 (EU Numbering) is a V185L substitution. In some embodiments, the HC CH1 substitution at C220 (EU Numbering) is a C220S substitution. In some embodiments, the LC CL1 substitution at position 124 (EU Numbering) is an E124C (lambda) or Q124C (kappa) substitution. In some embodiments, the LC CL1 substitution at position 124 (EU Numbering) is an E124C (lambda) substitution. In some embodiments, the LC CL1 substitution at position 124 (EU Numbering) is a Q124C (kappa) substitution. In some embodiments, the LC CL1 substitution at L135 (EU Numbering) is a L13SF substitution. In some embodiments, the LC CL1 substitution at C214 (EU Numbering) is a C214S substitution.

In some embodiments, the HC CH1 comprises or consists of an F126C, F170V, S183I, V185L, and C220S (EU Numbering) substitution, and the LC CL comprises or consists of an E124C, L135F, and C214S (lambda) or Q124C, L135F, and C214S (kappa) (EU Numbering) substitution. In some embodiments, the LC CL comprises or consists of an E124C, L135F, and C214S (lambda) (EU Numbering) substitution. In some embodiments, the LC CL comprises or consists of a Q124C, L135F, and C214S (kappa) (EU Numbering) substitution. In some embodiments, the HC CH1 comprises or consists of an F126C, F170I, S183L, V185L, and C220S (EU Numbering) substitution, and the LC CL comprises or consists of an E124C, L135F, and C214S (lambda) or Q124C, L135F, and C214S (kappa) (EU Numbering) substitution. In some embodiments, the LC CL comprises or consists of an E124C, L135F, and C214S (lambda) (EU Numbering) substitution. In some embodiments, the LC CL comprises or consists of a Q124C, L135F, and C214S (kappa) (EU Numbering) substitution. In some embodiments, the HC CH1 comprises or consists of SEQ ID NO: 15, 19, 23, or 24, and the LC CL comprises or consists of SEQ ID NO: 2, 5, 7, or 8. In some embodiments, the pharmaceutical composition is a cell culture. In some embodiments, the CH1 consists of substitutions at F9, F53, S66, V68, and C103, of SEQ ID NO: 9 and wherein the LC CL consists of a substitution at E18, L29, and C105 of SEQ ID NO: 1. In some embodiments, the CH1 consists of substitutions at F9, F53, S66, V68, and C103, of SEQ ID NO: 9, and wherein the LC CL consists of a substitution at Q17, L28, and C107 of SEQ ID NO: 6. In some embodiments, the HC CH1 substitution at F9 is an F9C substitution. In some embodiments, the HC CH1 substitution at F53 is an F53V or F53I substitution. In some embodiments, the HC CH1 substitution at S66 is a S66L, or S66I substitution. In some embodiments, the HC CH1 substitution at V68 is a V68L substitution. In some embodiments, the HC CH1 substitution at C103 is a C103S substitution. In some embodiments, the LC CL1 substitution at Q17 of SEQ ID NO: 6: is an Q17C substitution. In some embodiments, the LC CL1 substitution at E18 of SEQ ID NO: 1: is an E18C substitution. In some embodiments, the LC CL1 substitution at L28 of SEQ ID NO: 6 is a L28F substitution. In some embodiments, the LC CL1 substitution at L29 of SEQ ID NO: 1 is a L29F substitution. In some embodiments, the LC CL1 substitution at C107 of SEQ ID NO: 6 is a C107S substitution. In some embodiments, the LC CL1 substitution at C105 of SEQ ID NO: 1 is a C105S substitution. In some embodiments, the HC CH1 comprises or consists of an F9C, F53V, S661, V68L, and C103S substitution, and the LC CL comprises a Q17C, L28F, and a C107S substitution. In some embodiments, the HC CH1 comprises or consists of an F9C, F53V, S66I, V68L, and C103S substitution, and the LC CL comprises an E18C, L29F, and a C105S substitution at a position corresponding to F9, F53, S66, V68, and C103 of SEQ ID NO: 9 and E18, L29, and C105 of SEQ ID NO: 1. In some embodiments, the HC CH1 comprises or consists of an F9C, F53V, S66I, V68L, and C103S substitution, and the LC CL comprises a Q17C, L28F, and a C107S substitution at a position corresponding to F9, F53, S66, V68, and C103 of SEQ ID NO: 9 and Q17, L28, and C107 of SEQ ID NO: 6.

In some embodiments, the compositions disclosed herein comprise antibodies comprising two heavy chain/light chain pairs selected from the pairs of HC1/LC1, HC2/LC2, HC1/LC2 and HC2/LC1, wherein at least or at least about 50, 55, 60, 65, 70, 75, 80, 85, 90, 95, or 100% of the antibodies comprise the HC1/LC1 and HC2/LC2 pairs, or comprises a percentage of HC1/LC1 and HC2/LC2 pairs that is in a range defined by any two of the preceding values. For example, in some embodiments, the composition comprises antibodies comprising two heavy chain/light chain pairs selected from the pairs of HC1/LC1, HC2/LC2, HC1/LC2 and HC2/LC1, wherein between about 50-100, 50-95, 50-90, 50-80, 50-75, 50-70, 50-60, 50-55, 55-100, 55-95, 55-90, 55-80, 55-75, 55-70, 55-60, 60-100, 60-95, 6090, 60-80, 60-75, 60-70, 70-100, 70-95, 70-90, 70-80, 75-100, 75-95, 75-90, 75-80, 80-100, 80-95, 80-90, 90-100, 90-95, or 95-100%, of the antibodies comprise the HC1/LC1 and HC2/LC2 pairs.

In some embodiments, the compositions disclosed herein comprise none or not more than 0.1, 0.5, 1, 2, 3, 4, 5, 6, 7, 8, 9, 10, 15, 20, 25, 30, 35, 40, 45, or 50% of an antibody comprising either the HC1/LC2 or the HC2/LC1, or comprise a percentage of HC1/LC2 or the HC2/LC1 pairs that is in a range defined by any two of the preceding values. For example, in some embodiments, the compositions disclosed herein comprise between 0.150, 0.1-40, 0.1-20, 0.1-25, 0.1-20, 0.1-10, 0.1-5, 0.1-1, 1-50, 1-40, 1-30, 1-25, 1-20, 1-20, 1-5, 5-50, 5-40, 5-30, 5-25, 5-10, 10-50, 10-40, 10-25, 25-50, 25-40, 25-30, 30-50, 30-40, or 4050%, HC1/LC2 or the HC2/LC1 pairs.

In some embodiments, the compositions disclosed herein are a cell culture supernatant and/or cell lysate, and wherein the composition comprises at least 50, 55, 60, 65, 70, 75, 80, 85, 90, 95, or 100% antibodies comprising the HC1/LC1 and HC2/LC2 pairs prior to any enrichment of the composition for the antibodies comprising the HC1/LC1 and HC2/LC2 pairs, or comprise a percentage of HC1/LC1 and HC2/LC2 pairs that is in a range define by any two of the preceding values prior to any enrichment for HC1/LC1 and/or HC2/LC2 pairs. For example, in some embodiments, the compositions disclosed herein comprise between about 50-100, 50-95, 50-90, 50-80, 50-75, 50-70, 50-60, 50-55, 55-100, 5595, 55-90, 55-80, SS-75, 55-70, 55-60, 60-100, 60-95, 60-90, 60-80, 60-75, 60-70, 70-100, 70-95, 70-90, 70-80, 75-100, 75-95, 75-90, 75-80, 80-100, 80-95, 80-90, 90-100, 90-95, or 95100%, HC1/LC1 and/or HC2/LC2 pairs prior to any enrichment for HC1/LC1 and/or HC2/LC2 pairs.

In some embodiments disclosed herein, the composition comprises none or not more than 0.1, 0.5, 1, 2, 3, 4, 5, 6, 7, 8, 9, 10, 15, 20, 25, 30, 35, 40, 45, or 50% of an antibody or fragment thereof comprising either of the HC1/LC2 or the HC2/LC1 pairs prior to any enrichment of the composition for antibodies comprising the HC1/LC1 and HC2/LC2 pairs, or comprises a percentage of HC1/LC2 or the HC2/LC1 pairs that is in a range defined by any two of the preceding values prior to any enrichment of the composition for antibodies comprising the HC1/LC1 and HC2/LC2 pairs. For example, in some embodiments, the composition comprises between about 0.1-50, 0.1-40, 0.1-20, 0.1-25, 0.1-20, 0.1-10, 0.1-5, 0.1-1, 1-50, 1-40, 1-30, 1-25, 1-20, 1-20, 1-5, 5-50, 5-40, 5-30, 5-25, 5-10, 10-50, 10-40, 1025, 25-50, 25-40, 25-30, 30-50, 30-40, or 40-50%, HC1/LC2 or the HC2/LC1 pairs prior to any enrichment for HC1/LC1 and/or HC2/LC2 pairs. In some embodiments, the fragment comprises an antigen binding fragment.

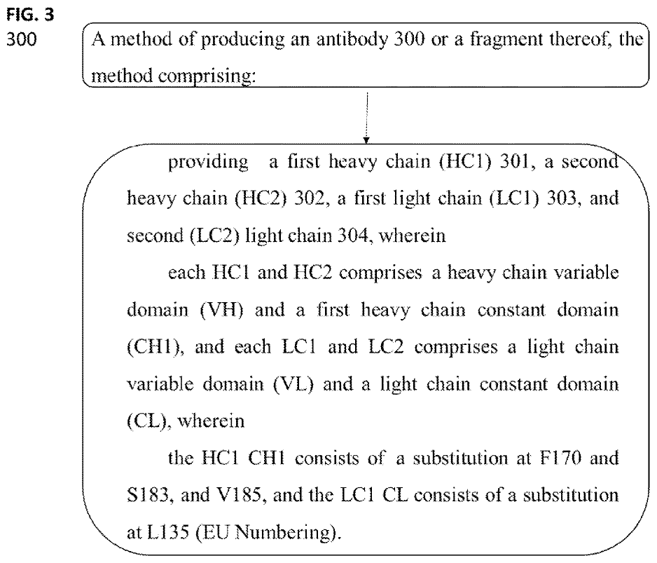

Disclosed herein are methods of producing antibodies or fragments thereof. FIG. 3 is a flow chart showing some embodiments of a method for producing an antibody or a fragment thereof. In some embodiments, the fragment comprises an antigen binding fragment. In some embodiments, the method of producing an antibody 300 or fragment thereof comprises: providing a first heavy chain (HC1) 301, a second heavy chain (HC2) 302, a first light chain (LC1) 303, and second (LC2) light chain 304, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG, wherein the HC1 CH1 comprises or consists of a substitution at F170 and S183, and optionally a substitution at V185 (EU Numbering), and the LC1 CL comprises or consists of a substitution at L135 (EU Numbering), wherein the HC1 CH1 does not include any substitution selected from L128F, A141M, A141T, A141I, F170M, F170Y, F170S, F170A, S181I, S181T, S181M, S183A, S183V, and V185A (EU Numbering), and wherein the LC1 CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (EU Numbering). In some embodiments, the HC1 CH1 comprises or consists of substitutions at F170 and S183 (EU Numbering), or at F170, S183 and V185 (EU Numbering), and wherein the LC1 CL comprises or consists of a substitution at L135 (EU Numbering). In some embodiments, the HC1 CH1 comprises a substitution at V185. In some embodiments, the HC1 CH1 substitution at F170 (EU Numbering) is an F170I, or F170V substitution. In some embodiments, the HC1 CH1 substitution at S183 (EU Numbering) is a S183L or S183I substitution. In some embodiments, the HC1 CH1 substitution at V185 (EU Numbering) is a V185L substitution. In some embodiments, the LC1 CL1 substitution at L135 (EU Numbering) is a L135F substitution. In some embodiments, the HC1 CH1 comprises or consists of an F1701 and S183V (EU Numbering) substitution, and the LC1 CL comprises or consists of a L135F (EU Numbering) substitution. In some embodiments, the HC1 CH1 comprises or consists of an F1701, S183L, and V185L (EU Numbering) substitution, and the LC1 CL comprises or consists of a L135F (EU Numbering) substitution. In some embodiments, the HC CH1 comprises a substitution at F53 and S66, and V68 of SEQ ID NO: 9, and the LC CL comprises a substitution at L29 of SEQ ID NO: 1 or L28 of SEQ ID NO: 6. In some embodiments, the HC CH1 substitution at F53 of SEQ ID NO: 9 is an F53I, or F53V substitution. In some embodiments, the HC CH1 substitution at S66 of SEQ ID NO: 9 is a S66L or S66I substitution. In some embodiments, the HC CH1 substitution at V68 of SEQ ID NO: 9 is a V68L substitution. In some embodiments, the LC CL substitution at L29 of SEQ ID NO: 1 or L28 of SEQ ID NO: 6 is a L29F or L28F substitution. In some embodiments, the antibody is a human IgG. In some embodiments, the antibody is a human IgG1, IgG2, IgG3, or IgG4, antibody. In some embodiments, the antibody is a chimeric antibody. In some embodiments, the antibody is a humanized antibody. In some embodiments, the antibody is a chimeric or humanized IgG1, IgG2, IgG3, or IgG4, antibody.

In some embodiments, the method of producing an antibody or a fragment thereof comprises providing a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG, wherein the HC1 CH1 comprises or consists of a substitution at F126, F170, S183, V185, and C220 (EU Numbering), and the LC1 CL comprises or consists of a substitution at E124, L135, and C214 (lambda) or Q124, L135, and C214 (kappa) (EU Numbering) (in some embodiments the LC CL comprises or consists of a substitution at E124, L135, and C214 (lambda); in some embodiments the LC CL comprises or consists of a substitution at Q124, L135, and C214 (kappa)), wherein the HC1 CH1 does not include any substitution selected from L128F, A141M, A141T, A141I, F170M, F170Y, F170S, F170A, S181I, S181T, S181M, S183A, S183V, and V185A (EU Numbering), and wherein the LC1 CL does not include any substitution selected from F116A, F118V, S131T, S131D, V133A, V133I, L13SY, L135V, S162A, S162M, T164S, S174A, S176M, S176A, S176T, S176F, T178L, T178V, and T178I (EU Numbering). In some embodiments, the fragment comprises an antigen binding fragment. In some embodiments, the HC CH1 substitution at F126 (EU Numbering) is an F126C substitution. In some embodiments, the HC CH1 substitution at F170 (EU Numbering) is an F170V or F170I substitution. In some embodiments, the HC CH1 substitution at S183 (EU Numbering) is a S183L, or S183I substitution. In some embodiments, the HC CH1 substitution at V185 (EU Numbering) is a V185L substitution. In some embodiments, the HC CH1 substitution at C220 (EU Numbering) is a C220S substitution. In some embodiments, the LC CL1 substitution at position 124 (EU Numbering) is an E124C (lambda) or Q124C (kappa) substitution. In some embodiments, the LC CL1 substitution at position 124 (EU Numbering) is an E124C (lambda) substitution. In some embodiments, the LC CL1 substitution at position 124 (EU Numbering) is a Q124C (kappa) substitution. In some embodiments, the LC CL1 substitution at L135 (EU Numbering) is a L135F substitution. In some embodiments, the LC CL1 substitution at C214 (EU Numbering) is a C214S substitution. In some embodiments, the HC CH1 comprises or consists of an F126C, F170V, S1831, V185L, and C220S (EU Numbering) substitution, and the LC CL comprises or consists of an E124C, L135F, and C214S (lambda) or Q124C, L135F, and C214S (kappa) (EU Numbering) substitution. In some embodiments, the LC CL comprises or consists of an E124C, L135F, and C214S (lambda) (EU Numbering) substitution. In some embodiments, the LC CL comprises or consists of a Q124C, L135F, and C214S (kappa) (EU Numbering) substitution. In some embodiments, the HC CH1 comprises an F126C, F170I, S183L, V185L, and C220S (EU Numbering) substitution, and the LC CL comprises or consists of an E124C, L135F, and C214S (lambda) or Q124C, L135F, and C214S (kappa) (EU Numbering) substitution. In some embodiments, the LC CL comprises or consists of an E124C, L135F, and C214S (lambda) (EU Numbering) substitution. In some embodiments, the LC CL comprises or consists of a Q124C, L135F, and C214S (kappa) (EU Numbering) substitution. In some embodiments, the antibody is a human IgG. In some embodiments, the antibody is a human IgG1, IgG2, IgG3, or IgG4, antibody. In some embodiments, the antibody is a chimeric antibody. In some embodiments, the antibody is a humanized antibody. In some embodiments, the antibody is a chimeric or humanized IgG1, IgG2, IgG3, or IgG4, antibody.

In some embodiments, the method of producing an antibody or a fragment thereof comprises providing a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG, wherein the HC1 CH1 comprises or consists of a substitution at F126 and C220 (EU Numbering), the LC1 CL comprises or consists of a substitution at position 124 (E124 for lambda and Q124 for kappa), and C214 (EU Numbering), the HC2 CH1 comprises or consists of a substitution at F170, S183, and V185 (EU Numbering), and the LC2 CL comprises or consists of a substitution L135 (EU Numbering), wherein the HC1 CH1 and/or HC2 CH1 does not include any substitution selected from L128F, A141M, A14IT, A141I, F170M, F170Y, F170S, F170A, S181I, S181T, S181M, S183A, S183V, and V185A (EU Numbering), and wherein the LC1 CL and/or LC2 CL does not include any substitution selected from F116A, F118V, S13IT, S131D, V133A, V133I, L135Y, L135V, S162A, S162M, T164S, S174A, S176M, S176A, S176T, S176F, T178L, T178V, and T178I (EU Numbering). In some embodiments, the fragment comprises an antigen binding fragment. In some embodiments, the HC1 CH1 substitution at F126 (EU Numbering) is an F126C substitution, and the LC1 CL substitution at position 124 (EU Numbering) is an E124C (lambda) or Q124C (kappa) substitution. In some embodiments, the LC CL1 substitution at position 124 (EU Numbering) is an E124C (lambda) substitution. In some embodiments, the LC CL1 substitution at position 124 (EU Numbering) is a Q124C (kappa) substitution. In some embodiments, the HC1 CH1 substitution at C220 (EU Numbering) is a C220S substitution. In some embodiments, the LC1 CL substitution at C214 (EU Numbering) is a C214S substitution. In some embodiments, the HC2 CH1 substitution at F170 (EU Numbering) is an F170V or F170I substitution. In some embodiments, the HC2 CH1 substitution at S183 (EU Numbering) is a S183L, or S183I substitution. In some embodiments, the HC2 CH1 substitution at V185 (EU Numbering) is a V185L substitution. In some embodiments, the LC2 CL substitution at L135 (EU Numbering) is a L135F substitution. In some embodiments, the HC1 CH1 comprises or consists of an F126C and C220S (EU Numbering) substitution, the LC1 CL comprises or consists of an E124C and C214S (lambda) or Q124C and C214S (kappa) (EU Numbering) substitution (in some embodiments, the LC CL comprises or consists of an E124C and C214S (lambda) substitution; in some embodiments, the LC CL comprises or consists of a Q124C and C214S (kappa) substitution), the HC2 CH1 comprises or consists of an F170I, S183L, V185L (EU Numbering), and the LC2 CL comprises or consists of a L135F (EU Numbering) substitution. In some embodiments, the HC1 CH1 comprises or consists of an F126C and C220S (EU Numbering) substitution, the LC1 CL comprises or consists of an E124C and C214S (lambda) or Q124C and C214S (kappa) (EU Numbering) substitution (in some embodiments, the LC CL comprises or consists of an E124C and C214S (lambda) substitution, in some embodiments, the LC CL comprises or consists of a Q124C and C214S (kappa) substitution), the HC2 CH1 comprises or consists of an F170V, S183I, V18SL (EU Numbering), and the LC2 CL comprises or consists of a L135F (EU Numbering) substitution. In some embodiments, the CH1 consists of substitutions at F9, F53, S66, V68, and C103, of SEQ ID NO: 9 and wherein the LC CL consists of a substitution at E18, L29, and C105 of SEQ ID NO: 1. In some embodiments, the CH1 consists of substitutions at F9, F53, S66, V68, and C103, of SEQ ID NO: 9, and wherein the LC CL consists of a substitution at Q17, L28, and C107 of SEQ ID NO: 6. In some embodiments, the HC CH1 substitution at F9 is an F9C substitution. In some embodiments, the HC CH1 substitution at F53 is an F53V or F53I substitution. In some embodiments, the HC CH1 substitution at S66 is a S66L, or S66I substitution. In some embodiments, the HC CH1 substitution at V68 is a V68L substitution. In some embodiments, the HC CH1 substitution at C103 is a C103S substitution. In some embodiments, the LC CL1 substitution at Q17 of SEQ ID NO: 6: is an Q17C substitution. In some embodiments, the LC CL1 substitution at E18 of SEQ ID NO: 1: is an E18C substitution. In some embodiments, the LC CL1 substitution at L28 of SEQ ID NO: 6 is a L28F substitution. In some embodiments, the LC CL1 substitution at L29 of SEQ ID NO: 1 is a L29F substitution. In some embodiments, the LC CL1 substitution at C107 of SEQ ID NO: 6 is a C107S substitution. In some embodiments, the LC CL1 substitution at C105 of SEQ ID NO: 1 is a C105S substitution. In some embodiments, the HC CH1 comprises or consists of an F9C, F53V, S66I, V68L, and C103S substitution, and the LC CL comprises a Q17C, L28F, and a C107S substitution. In some embodiments, the HC CH1 comprises or consists of an F9C, F53V, S66I, V68L, and C103S substitution, and the LC CL comprises an E18C, L29F, and a C105S substitution at a position corresponding to F9, F53, S66, V68, and C103 of SEQ ID NO: 9 and E18, L29, and C105 of SEQ ID NO: 1. In some embodiments, the HC CH1 comprises or consists of an F9C, F53V, S66I, V68L, and C103S substitution, and the LC CL comprises a Q17C, L28F, and a C107S substitution at a position corresponding to F9, F53, S66, V68, and C103 of SEQ ID NO: 9 and Q17, L28, and C107 of SEQ ID NO: 6. In some embodiments, the antibody is a human IgG. In some embodiments, the antibody is a human IgG1, IgG2, IgG3, or IgG4, antibody. In some embodiments, the antibody is a chimeric antibody. In some embodiments, the antibody is a humanized antibody. In some embodiments, the antibody is a chimeric or humanized IgG1, IgG2, IgG3, or IgG4, antibody.

In some embodiments, the method of producing an antibody or a fragment thereof comprises providing a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG, wherein the LC VL framework region (FR) and/or the HC VH framework region (FR) comprises one or more substitutions. In some embodiments, the VL FR comprises a substitution at Q38, and/or G100, and/or G101 (EU Numbering). In some embodiments, the VH FR comprises a substitution at Q39, and/or G44 (EU Numbering). In some embodiments, the VH FR comprises a substitution at Q39, and/or G44 (EU Numbering), and the VL FR comprises a substitution at Q38, and/or G100, and/or G101 (EU Numbering). In some embodiments, the VL FR substitution at Q38 comprises or consists of a substitution selected from the group consisting of a Q38D and a Q38K substitution (EU Numbering). In some embodiments, the VL FR substitution at G100 comprises or consists of a substitution selected from the group consisting of a G100D, a G100K, and a G100C substitution (EU Numbering). In some embodiments, the VL FR substitution at G101 comprises or consists of a substitution selected from the group consisting of G101D and G101K substitution (EU Numbering). In some embodiments, the VH FR substitution at Q39 comprises or consists of a substitution selected from the group consisting of a Q39D and a Q39K substitution (EU Numbering). In some embodiments, the VH FR substitution at G44 comprises or consists of a substitution selected from the group consisting of a G44K, a G44D, and a G44C substitution (EU Numbering). In some embodiments, the substitution of the VH FR comprises or consists of a Q39K substitution, and the substitution of the VL FR comprises or consists of a Q38D substitution. In some embodiments, the substitution of the VH FR comprises or consists of a Q39D substitution, and the substitution of the VL FR comprises or consists of a Q38K substitution. In some embodiments, the substitution of VH FR comprises or consists of a G44K substitution, and the substitution of the VL FR comprises or consists of a G100D substitution. In some embodiments, the substitution of the VH FR comprises or consists of a G44D substitution, and the substitution of the VL FR comprises or consists of a G100K substitution. In some embodiments, the substitution of the VH FR comprises or consists of a G44C substitution, and the substitution of the VL FR comprises or consists of a G100C substitution. In some embodiments, the substitution of the VH FR comprises or consists of a G44K substitution, and the substitution of the VL FR comprises or consists of a G101D substitution. In some embodiments, the substitution of the VH FR comprises or consists of a G44D substitution, and the substitution of the VL FR comprises or consists of a G101K substitution. In some embodiments, the substitution of the HC1 VH FR comprises or consists of a Q39K substitution, the substitution of the LC1 VL FR comprises or consists of a Q38D substitution, the substitution of the HC2 VH FR comprises or consists of a Q39D substitution, and the substitution of the LC2 VL FR comprises or consists of a Q38K substitution. In some embodiments, the substitution of the HC1 VH FR comprises or consists of a Q39D substitution, the substitution of the LC1 VL FR comprises or consists of a Q38K substitution, the substitution of the HC2 VH FR comprises or consists of a Q39K substitution, and the substitution of the LC2 VL FR comprises or consists of a Q38D substitution. In some embodiments, the substitution of the HC1 VH FR comprises or consists of a G44K substitution, the substitution of the LC1 VL FR comprises or consists of a G100D substitution, the substitution of the HC2 VH FR comprises or consists of a G44D substitution, and the substitution of the LC2 VL FR comprises or consists of a G100K substitution. In some embodiments, the substitution of the HC1 VH FR comprises or consists of a G44D substitution, the substitution of the LC1 VL FR comprises or consists of a G100K substitution, the substitution of the HC2 VH FR comprises or consists of a G44K substitution, and the substitution of the LC2 VL FR comprises or consists of a G100D substitution. In some embodiments, the substitution of the HC1 VH FR comprises or consists of a G44C substitution and the substitution of the LC1 VL FR comprises or consists of a G100C substitution. In some embodiments, the substitution of the HC2 VH FR comprises or consists of a G44C substitution and the substitution of the LC1 VL FR comprises or consists of a G100C substitution. In some embodiments, the substitution of HC1 VH FR comprises or consists of a G44K substitution, the substitution of the LC1 VL FR comprises or consists of a G101D substitution, the substitution of the HC2 VH FR comprises or consists of a G44D substitution, and the substitution of the LC2 VL FR comprises or consists of a G101K substitution. In some embodiments, the substitution of HC1 VH FR comprises or consists of a G44D substitution, the substitution of the LC1 VL FR comprises or consists of a G101K substitution, the substitution of the HC2 VH FR comprises or consists of a G44K substitution, and the substitution of the LC2 VL FR comprises or consists of a G101D substitution.

In some embodiments, the method of producing an antibody or a fragment thereof comprises providing a first heavy chain (HC1), a second heavy chain (HC2), a first light chain (LC1), and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG, wherein the LC VL framework region (FR) and/or the HC VH framework region (FR) comprises one or more substitutions. In some embodiments, the HC1 or HC2 VH comprises or consists of any one of SEQ ID NO: 53-64. In some embodiments, the LC1 or LC2 VL comprises or consists of any one of SEQ ID NO: 65-80. In some embodiments, the heavy chain and light chain of the HC1/LC1 pair and the heavy chain and light chain of the HC2/LC2 pair each comprise or consist of the following sequence, respectively: SEQ ID NOs: 30, 43, 35 and 52; or SEQ ID NOs: 35, 52, 30 and 43. In some embodiments, the heavy chain and light chain of the HC1/LC1 pair and the heavy chain and light chain of the HC2/LC2 pair each comprise or consist of the following sequence, respectively: SEQ ID NOs: 29, 44, 36 and 51; or SEQ ID NOs: 36, 51, 29 and 44. In some embodiments, the heavy chain and light chain of the HC1/LC1 pair and the heavy chain and light chain of the HC2/LC2 pair each comprise or consist of the following sequence, respectively: SEQ ID NOs: 28, 39, 33 and 48; or SEQ ID NOs: 33, 48, 28 and 39. In some embodiments, the heavy chain and light chain of the HC1/LC1 pair and the heavy chain and light chain of the HC2/LC2 pair each comprise or consist of the following sequence, respectively: SEQ ID NOs: 27, 40, 34 and 47; or SEQ ID NOs: 34, 47, 27 and 40. In some embodiments, the heavy chain and light chain of the HC1/LC1 pair and the heavy chain and light chain of the HC2/LC2 pair each comprise or consist of the following sequence, respectively: SEQ ID NOs: 26, 38, 31 and 45; or SEQ ID NOs: 31, 45, 26 and 38. In some embodiments, the heavy chain and light chain of the HC1/LC1 pair and the heavy chain and light chain of the HC2/LC2 pair each comprise or consist of the following sequence, respectively: SEQ ID NOs: 25, 37, 32 and 46; or SEQ ID NOs: 32, 46, 25 and 37. In some embodiments, the heavy chain and light chain of the HC1/LC1 pair and the heavy chain and light chain of the HC2/LC2 pair each comprise or consist of the following sequence, respectively: SEQ ID NOs: 28, 41, 33 and 50; or SEQ ID NOs: 33, 50, 28 and 41. In some embodiments, the heavy chain and light chain of the HC1/LC1 pair and the heavy chain and light chain of the HC2/LC2 pair each comprise or consist of the following sequence, respectively: SEQ ID NOs: 27, 42, 34 and 49; or SEQ ID NOs: 34, 49, 27 and 42. In some embodiments, the HC1 comprises or consists of SEQ ID NO: 30, the LC1 comprises or consists of SEQ ID NO: 43, the HC2 comprises or consists of SEQ ID NO: 35, and the LC2 comprises or consists of SEQ ID NO: 52. In some embodiments, the HC1 comprises or consists of SEQ ID NO: 29, the LC1 comprises or consists of SEQ ID NO: 44, the HC2 comprises or consists of SEQ ID NO: 36, and the LC2 comprises or consists of SEQ ID NO: 51. In some embodiments, the HC1 comprises or consists of SEQ ID NO: 28, the LC1 comprises or consists of SEQ ID NO: 39, the HC2 comprises or consists of SEQ ID NO: 33, and the LC2 comprises or consists of SEQ ID NO: 48. In some embodiments, the HC1 comprises or consists of SEQ ID NO: 27, the LC1 comprises or consists of SEQ ID NO: 40, the HC2 comprises or consists of SEQ ID NO: 34, and the LC2 comprises or consists of SEQ ID NO: 47. In some embodiments, the HC1 comprises or consists of SEQ ID NO: 26, the LC1 comprises or consists of SEQ ID NO: 38, the HC2 comprises or consists of SEQ ID NO: 31, and the LC2 comprises or consists of SEQ ID NO: 45. In some embodiments, the HIC1 comprises or consists of SEQ ID NO: 25, the LC1 comprises or consists of SEQ ID NO: 37, the HC2 comprises or consists of SEQ ID NO: 32, and the LC2 comprises or consists of SEQ ID NO: 46. In some embodiments, the HC1 comprises or consists of SEQ ID NO: 28, the LC1 comprises or consists of SEQ ID NO: 39, the HC2 comprises or consists of SEQ ID NO: 33, and the LC2 comprises or consists of SEQ ID NO: 50. In some embodiments, the HC1 comprises or consists of SEQ ID NO: 27, the LC1 comprises or consists of SEQ ID NO: 40, the HC2 comprises or consists of SEQ ID NO: 34, and the LC2 comprises or consists of SEQ ID NO: 49. In some embodiments, the HC1 comprises or consists of SEQ ID NO: 35, the LC1 comprises or consists of SEQ ID NO: 52, the HC2 comprises or consists of SEQ ID NO: 30, and the LC2 comprises or consists of SEQ ID NO: 43. In some embodiments, the HC1 comprises or consists of SEQ ID NO: 36, the LC1 comprises or consists of SEQ ID NO: 51, the HC2 comprises or consists of SEQ ID NO: 29, and the LC2 comprises or consists of SEQ ID NO: 44. In some embodiments, the HC1 comprises or consists of SEQ ID NO: 33, the LC1 comprises or consists of SEQ ID NO: 48, the HC2 comprises or consists of SEQ ID NO: 28, and the LC2 comprises or consists of SEQ ID NO: 39. In some embodiments, the HC1 comprises or consists of SEQ ID NO: 34, the LC1 comprises or consists of SEQ ID NO: 47, the HC2 comprises or consists of SEQ ID NO: 27, and the LC2 comprises or consists of SEQ ID NO: 40. In some embodiments, the HC1 comprises or consists of SEQ ID NO: 31, the LC1 comprises or consists of SEQ ID NO: 45, the HC2 comprises or consists of SEQ ID NO: 26, and the LC2 comprises or consists of SEQ ID NO: 38. In some embodiments, the HC1 comprises or consists of SEQ ID NO: 32, the LC1 comprises or consists of SEQ ID NO: 46, the HC2 comprises or consists of SEQ ID NO: 25, and the LC2 comprises or consists of SEQ ID NO: 37. In some embodiments, the HC1 comprises or consists of SEQ ID NO: 33, the LC1 comprises or consists of SEQ ID NO: 50, the HC2 comprises or consists of SEQ ID NO: 28, and the LC2 comprises or consists of SEQ ID NO: 41. In some embodiments, the HC1 comprises or consists of SEQ ID NO: 34, the LC1 comprises or consists of SEQ ID NO: 49, the HC2 comprises or consists of SEQ ID NO: 27, and the LC2 comprises or consists of SEQ ID NO: 42.

In some embodiments, LC-HC pairing fidelity in bispecific IgG1 molecules, optionally when both Fabs are with kappa LCs as light chains, pairing mutations are introduced to VL-CL interface in addition to the CL-CH1 pairing mutations F/JT7 or F/JT11. In some embodiments, residue pairs that are on VL-VH interface are mutated to Cys for disulfide bond formation, or to opposite charge residues for electrostatic interactions, optionally as shown in Table 4. In some embodiments, the antibody or binding fragment thereof comprises or consists of one or more of the combinations of substitutions shown in Table 4. For example, in some embodiments, the antibody or binding fragment thereof comprises a VH comprising or consisting of a Q39K, Q39D, G44K, G44D, or G44C substitution. In some embodiments, the antibody or binding fragment thereof comprises a CH1 comprising or consisting of a F170V, S183I, or V185L substitution. In some embodiments, the antibody or binding fragment thereof comprises a VL comprising or consisting of a Q38D, Q38K, G100D, G100K, or G100C substitution. In some embodiments, the antibody or antigen binding fragment thereof comprises a CL comprising or consisting of a L135F substitution. In some embodiments, the antibody or binding fragment thereof comprises or consists of the combinations of substitutions listed for the First Fab (Cytokine C Fab) of any one of V1, V2, V3, V4, V5, V6, V7 or V8 of Table 4. In some embodiments, the antibody or binding fragment thereof comprises or consists of the combinations of substitutions listed for the Second Fab (Cytokine D Fab) of any one of V1, V2, V3, V4, V5, V6, V7 or V8 of Table 4. In some embodiments, the antibody or binding fragment thereof comprises or consists of the combinations of substitutions listed for the First Fab and the Second Fab of any one of V1, V2, V3, V4, V5, V6, V7 or V8 of Table 4. In some embodiments, antibody or binding fragment thereof comprises or consists of the combination of substitutions listed for the First Fab and/or the Second Fab of V1 of Table 4. In some embodiments, antibody or binding fragment thereof comprises or consists of the combination of substitutions listed for the First Fab and/or the Second Fab of V2 of Table 4. In some embodiments, antibody or binding fragment thereof comprises or consists of the combination of substitutions listed for the First Fab and/or the Second Fab of V3 of Table 4. In some embodiments, antibody or binding fragment thereof comprises or consists of the combination of substitutions listed for the First Fab and/or the Second Fab of V4 of Table 4. In some embodiments, antibody or binding fragment thereof comprises or consists of the combination of substitutions listed for the First Fab and/or the Second Fab of V5 of Table 4. In some embodiments, antibody or binding fragment thereof comprises or consists of the combination of substitutions listed for the First Fab and/or the Second Fab of V6 of Table 4. In some embodiments, antibody or binding fragment thereof comprises or consists of the combination of substitutions listed for the First Fab and/or the Second Fab of V7 of Table 4. In some embodiments, antibody or binding fragment thereof comprises or consists of the combination of substitutions listed for the First Fab and/or the Second Fab of V8 of Table 4. In some embodiments, the antibody or antigen binding fragment thereof comprises a first Fab comprising a VH comprising a Q39K substitution, a wild-type CH1, a VL comprising or consisting of a Q38D substitution, and a wild-type CL, and/or a second Fab comprising a VH comprising a Q39D substitution, a CH1 comprising or consisting of a F170V, S1831, and/or V185L substitution; a VL comprising or consisting of a Q38K substitution, and a CL comprising or consisting of a L135F substitution. In some embodiments, the antibody or antigen binding fragment thereof comprises a first Fab comprising a VH comprising a Q39D substitution, a wild-type CH1, a VL comprising or consisting of a Q38K substitution, and a wild-type CL, and/or a second Fab comprising a VH comprising a Q39K substitution, a CH1 comprising or consisting of a F170V, S183I, and/or V185L substitution; a VL comprising or consisting of a Q38D substitution, and a CL comprising or consisting of a L135F substitution. In some embodiments, the antibody or antigen binding fragment thereof comprises a first Fab comprising a VH comprising a G44K substitution, a wild-type CH1, a VL comprising or consisting of a G100D substitution, and a wild-type CL, and/or a second Fab comprising a VH comprising a G44D substitution, a CH1 comprising or consisting of a F170V, S183I, and/or V185L substitution; a VL comprising or consisting of a G100K substitution, and a CL comprising or consisting of a L135F substitution. In some embodiments, the antibody or antigen binding fragment thereof comprises a first Fab comprising a VH comprising a G44D substitution, a wild-type CH1, a VL comprising or consisting of a G100K substitution, and a wild-type CL, and/or a second Fab comprising a VH comprising a G44K substitution, a CH1 comprising or consisting of a F170V, S183I, and/or V185L substitution; a VL comprising or consisting of a G100D substitution, and a CL comprising or consisting of a L135F substitution. In some embodiments, the antibody or antigen binding fragment thereof comprises a first Fab comprising a VH comprising a G44C substitution, a wild-type CH1, a VL comprising or consisting of a G100C substitution, and a wild-type CL, and/or a second Fab comprising a wild-type VH, a CH1 comprising or consisting of a F170V, S1831, and/or V185L substitution; a wild-type VL, and a CL comprising or consisting of a L135F substitution. In some embodiments, the antibody or antigen binding fragment thereof comprises a first Fab comprising a wild-type VH, a wild-type CH1, a wild-type VL, and a wild-type CL, and/or a second Fab comprising a VH comprising a G44C substitution, a CH1 comprising or consisting of a F170V, S1831, and/or V185L substitution, a VL comprising or consisting of a G100C substitution, and a CL comprising or consisting of a L135F substitution. In some embodiments, the antibody or antigen binding fragment thereof comprises a first Fab comprising a VH comprising a G44K substitution, a wild-type CH1, a VL comprising or consisting of a G101D substitution, and a wild-type CL, and/or a second Fab comprising a VH comprising a G44D substitution, a CH1 comprising or consisting of a F170V, S183I, and/or V185L substitution; a VL comprising or consisting of a G101K substitution, and a CL comprising or consisting of a L135F substitution. In some embodiments, the antibody or antigen binding fragment thereof comprises a first Fab comprising a VH comprising a G44D substitution, a wild-type CH1, a VL comprising or consisting of a G101K substitution, and a wild-type CL, and/or a second Fab comprising a VH comprising a G44K substitution, a CH1 comprising or consisting of a F170V, S1831, and/or V185L substitution; a VL comprising or consisting of a G10ID substitution, and a CL comprising or consisting of a L13SF substitution. In some embodiments, pairing for the HCs in the bispecific IgGs are conveyed in the hole mutations (T366S/S368A/Y407V) in the HC of first Fab (e.g., Cytokine C) and the knob mutation (T366W) in the HC of second Fab (e.g., Cytokine D).

In some embodiments, the four chains (HC1, HC2, LC1 and LC2) are transfected in a cell for expression at a ratio of 1:1:1:1. In some embodiments, the four chains (HC1, HC2, LC1 and LC2) are transfected in a cell for expression at a ratio of 1:1:2:1. In some embodiments, the four chains (HC1, HC2, LC1 and LC2) are transfected in a cell for expression at a ratio of 1:1:5:1. In some embodiments, the four chains (HC1, HC2, LC1 and LC2) are transfected in a cell for expression at a ratio of 1:1:1.5:1.5. In some embodiments, the four chains (HC1, HC2, LC1 and LC2) are transfected in a cell for expression at a ratio of wherein the amount of HC1, HC2 and LC2 are, or are about, the same, and the amount of LC1 is or is about 1, 1.5, 2, 2.5, 3, 3.5, 4, 4.5, 5, 5.5, 6, 6.5, 7, 7.5, 8, 8.5, 8, 9.5, 10 or greater relative to the HC1, HC2 and LC2. In some embodiments, the cell is an Expi293 cell. In some embodiments, the expressed IgG molecules in conditioned media are captured on a Protein A resin (e.g., MabSelect SuRe™). In some embodiments, Protein A capture removes non-product related impurities. In some embodiments, after non-specific bindings are washed off with PBS (phosphate buffered saline) solution, the expressed antibodies are eluted, optionally with 0.5% acetic acid, pH 3.5 and neutralized with 15% volumes of 1 M Tris-HC1, pH 8.0 to a final pH of 7.4. In some embodiments, the antibody pools are further polished. In some embodiments, polishing chromatography removes product-related and residual impurities. In some embodiments, the antibodies are polished, for example by using mixed mode chromatography. In some embodiments, multimodal cation exchanger (e.g., Capto MMC ImpRes™, CMM HyperCel, CIMmultus® PrimaS, CIMmultus® H-Bond) is used to purify the correctly paired antibodies or biding fragments thereof (e.g., Fabs). In some embodiments, HCs and LCs of bispecific antibodies, or antigen binding fragments thereof, pair correctly in an amount (e.g., a percentage) that is, is about (e.g., ±1, 5, or 10% of the value) or is at least, a value depicted in Table 5. In some embodiments, the percentage of correctly assembled bispecific IgG1 is calculated by quantifying all IgG1 species present (excluding half antibodies and homodimers of half antibodies (see, e.g., Table 5)). For example, in some embodiments, an antibody or antigen binding fragment thereof comprising a cytokine C Fab comprising a VH comprising or consisting of a Q39K, Q39D, G44C, or G44K substitution, a wild-type CH1, a VL comprising or consisting of a Q38D, Q38K, G100C, or G101D substitution, and a wild-type CL; and a cytokine D Fab comprising a VH comprising or consisting of a Q39D, Q39K, G44C, or G44D substitution, a CH1 comprising or consisting of a F170V, S183I, and/or V185L substitution; a VL comprising or consisting of a Q38K, Q38D, G100C, or G101K substitution, and a CL comprising or consisting of a L135F substitution, pairs correctly between about 60-100% of the time. In some embodiments, a bispecific antibody, or binding fragment thereof, comprises the combination of substitution listed in Table 4 for V1, and the HCs and LCs of the bispecific antibody pair correctly in an amount that is, is about, or is at least, 55%, 60% or 65%, optionally 55-65%. In some embodiments, a bispecific antibody, or binding fragment thereof, comprises the combination of substitution listed in Table 4 for V2, and the HCs and LCs of the bispecific antibody pair correctly in an amount that is, is about, or is at least, 65%, 70% or 75%, optionally 65-75%. In some embodiments, a bispecific antibody, or binding fragment thereof, comprises the combination of substitution listed in Table 4 for V5, and the HCs and LCs of the bispecific antibody pair correctly in an amount that is, is about, or is at least, 90%, 95%, 99% or 100%, optionally 90-100%. In some embodiments, a bispecific antibody, or binding fragment thereof, comprises the combination of substitution listed in Table 4 for V6, and the HCs and LCs of the bispecific antibody pair correctly in an amount that is, is about, or is at least, 85%, 90% or 95%, optionally 85-95%. In some embodiments, a bispecific antibody, or binding fragment thereof, comprises the combination of substitution listed in Table 4 for V7, and the HCs and LCs of the bispecific antibody pair correctly in an amount that is, is about, or is at least, 45%, 50% or 55%, optionally 45-55%. In some embodiments, purification, e.g., polishing on a mixed modal column (e.g., Capto MMC ImpRes™™) removes mispaired contaminants and increases the amount (e.g., percent) of correctly paired bispecific antibody by an amount that is, or is at least, 5%, 10%, 15%, 20%, 25% or 30%, optionally 5-30%. In some embodiments, correct pairing is determined using liquid chromatography-mass spectrometry (LC-MS). In some embodiments, antigen binding fragments of a bispecific antibody, pair correctly in an amount (e.g., a percentage) that is, is about (e.g., ±1, 5, or 10% of the value) or is at least, a value depicted in Table 6. For example, in some embodiments, an antibody or antigen binding fragment thereof comprising a cytokine C Fab comprising a VH comprising or consisting of a Q39K, Q39D, G44C, or G44K substitution, a wild-type CH1, a VL comprising or consisting of a Q38D, Q38K, G100C, or G101D substitution, and a wild-type CL; and a cytokine D Fab comprising a VH comprising or consisting of a Q39D, Q39K, G44C, or G44D substitution, a CH1 comprising or consisting of a F170V, S1831, and/or V185L substitution; a VL comprising or consisting of a Q38K, Q38D, G100C, or G101K substitution, and a CL comprising or consisting of a L135F substitution; pairs correctly between about 80-100% of the time. In some embodiments, binding fragments of a bispecific antibody comprises the combination of substitution listed in Table 4 for V1, and the HCs and LCs of the bispecific antibody pair correctly in an amount that is, is about, or is at least, 80%, 85% or 90%, optionally 80-90%. In some embodiments, a bispecific antibody, or binding fragment thereof, comprises the combination of substitution listed in Table 4 for V2, and the HCs and LCs of the bispecific antibody pair correctly in an amount that is, is about, or is at least, 80%, 85% or 90%, optionally 80-90%. In some embodiments, a bispecific antibody, or binding fragment thereof, comprises the combination of substitution listed in Table 4 for V5, and the HCs and LCs of the bispecific antibody pair correctly in an amount that is, is about, or is at least, 90%, 95%, 99% or 100%, optionally 90-100%. In some embodiments, a bispecific antibody, or binding fragment thereof, comprises the combination of substitution listed in Table 4 for V6, and the HCs and LCs of the bispecific antibody pair correctly in an amount that is, is about, or is at least, 90%, 95%, 99% or 100%, optionally 90-100%. In some embodiments, a bispecific antibody, or binding fragment thereof, comprises the combination of substitution listed in Table 4 for V7, and the HCs and LCs of the bispecific antibody pair correctly in an amount that is, is about, or is at least, 80%, 85% or 90%, optionally 80-90%. In some embodiments, purification, e.g., polishing on a mixed modal column (e.g., Capto MMC ImpRes™™) removes mispaired contaminants and increases the amount (e.g., percent) of correctly paired bispecific antibody by an amount that is, or is at least, 5%, 10%, 15%, 20%, 25% or 30%, optionally 5-30%. In some embodiments, correct pairing is determined using liquid chromatography-mass spectrometry (LC-MS).

In some embodiments, antibodies heavy chains or antigen binding fragments thereof pair correctly a percentage of the time as depicted in Table 7. For example, in some embodiments, an antibody or antigen binding fragment thereof comprising a First Fab and/or a Second Fab pairs correctly between about 60-100% of the time when transfected with DNA encoding First Fab and Second Fab light chains at a ratio of 1:1, 2:1, or 5:1. In some embodiments, the first HC and second HC are present in equal amounts, and the ratio of HC1 HC2, LC1, LC2 is 1:1:1.5:1.5, 1:1:2:1, and 1:1:2.5:0.5. In some embodiments, correct pairing of a First Fab (HC1/LC1) can be increased by increasing the ratio of First Fab (LC1) to Second Fab (LC2) light chains from 1:1 to 2:1 or 5:1. In some embodiments, increasing the ratio of First Fab (LC1) to Second Fab (LC2) light chains from 1:1 to 2:1 or 5:1 removes up to about 80-100% of HC1/LC2 mispairs. In some embodiments, increasing the ratio of LC1 to LC2 results in increased misparing (HC2/LC1). In some embodiments, purification, e.g., polishing on a mixed modal column (e.g., Capto MMC ImpRes™™) removes between up to about 80-100% of HC2/LC1 mispairing contaminants.

In some embodiments, a plurality of antibodies comprising two heavy chain/light chain pairs are produced, and at least 50, 55, 60, 65, 70, 75, 80, 85, 90, 95, or 100% of the antibodies comprising two heavy chain/light chain pairs that are produced comprise HC1/LC1 and HC2/LC2, or a percentage of antibodies comprising HC1/LC1 and HC2/LC2 pairs in a range defined by any two of the preceding values is produced. For example, in some embodiments, between about 50-100, 50-95, 50-90, 50-80, 50-75, 50-70, 50-60, 50-55, 55100, 55-95, 55-90, SS-80, 55-75, 55-70, 55-60, 60-100, 60-95, 60-90, 60-80, 60-75, 60-70, 70100, 70-95, 70-90, 70-80, 75-100, 75-95, 75-90, 75-80, 80-100, 80-95, 80-90, 90-100, 90-95, or 95-100%, of the produced antibodies comprise HC1/LC1 and HC2/LC2 pairs.

In some embodiments, none or not more than 0.1, 0.5, 1, 2, 3, 4, 5, 6, 7, 8, 9, 10, 15, 20, 25, 30, 35, 40, 45, or 50% of the antibodies comprising two heavy chain/light chain pairs that are produced comprise either a HC1/LC2 or a HC2/LC1, or comprises a percentage of HC1/LC2 or the HC2/LC1 pairs that is in a range defined by any two of the preceding values. For example, in some embodiments, the composition comprises between about 0.1-50, 0.1-40, 0.1-20, 0.1-25, 0.1-20, 0.1-10, 0.1-5, 0.1-1, 1-50, 1-40, 1-30, 1-25, 1-20, 1-20, 1-5, 5-50, 5-40, 5-30, 5-25, 5-10, 10-50, 10-40, 10-25, 25-50, 25-40, 25-30, 30-50, 3040, or 40-50%, HC1/LC2 or the HC2/LC1 pairs. In some embodiments, the percentage of the antibodies comprising HC1/LC1 and HC2/LC2 pairs refers to the percentage of HC1/LC1 and HC2/LC2 pairs prior to any enrichment for antibodies comprising the HC1/LC1 and HC2/LC2 pairs. In some embodiments, the percentage (%) of the antibodies comprising HC1/LC2 and HC2/LC1 pairs refers to the percentage of HC1/LC2 and HC2/LC1 pairs prior to any enrichment for antibodies comprising the HC1/LC1 and HC2/LC2 pairs.

In some embodiments, the providing comprises providing one or more polynucleotides encoding the HC1, HC2, LC1, and LC2. In some embodiments, the one or more polynucleotides encodes an IgG Fab or antibody. In some embodiments, the polynucleotide encodes a human IgG. In some embodiments, the polynucleotide encodes a human IgG1, IgG2, IgG3, or IgG4, antibody. In some embodiments, the polynucleotide encodes a chimeric antibody. In some embodiments, the polynucleotide encodes a humanized antibody. In some embodiments, the polynucleotide encodes a chimeric or humanized IgG1, IgG2, IgG3, or IgG4, antibody.

In some embodiments, the providing comprises providing an expression vector or construct encoding the HC1, HC2, LC1, and LC2. In some embodiments, the expression vector or construct encodes the HC1, HC2, LC1, and LC2 at a ratio of 1:1:1:1, 1:1:2:1, or 1:1:5:1. In some embodiments, the expression vector is a plasmid or viral vector. In some embodiments, the expression vector or construct encodes an IgG Fab or antibody. In some embodiments, the expression vector or construct expresses a polynucleotide encoding a human antibody or fragment thereof. In some embodiments, the fragment comprises an antigen binding fragment. In some embodiments, the expression vector or construct expresses a polynucleotide encoding a human IgG. In some embodiments, the expression vector or construct expresses a polynucleotide encoding a human IgG1, IgG2, IgG3, or IgG4, antibody. In some embodiments, the expression vector or construct expresses a polynucleotide encoding a chimeric antibody. In some embodiments, the expression vector or construct expresses a polynucleotide encoding a humanized antibody. In some embodiments, the expression vector or construct expresses a polynucleotide encoding a chimeric or humanized IgG1, IgG2, IgG3, or IgG4, antibody.

In some embodiments, the providing comprises providing a cell expressing the HC1, HC2, LC1, and LC2. In some embodiments, the cell expresses an IgG Fab or antibody. In some embodiments, the cell is a mammalian cell or bacterial cell. In some embodiments, the cell is a HEK293 cell, Chinese hamster ovary (CHO) cell, or a NS0 cell. In some embodiments, the cell comprises one or more polynucleotides, expression vectors, or constructs, encoding a human antibody or fragment thereof. In some embodiments, the fragment comprises an antigen binding fragment. In some embodiments, the cell comprises one or more polynucleotides, expression vectors, or constructs, encoding a human IgG. In some embodiments, the cell comprises one or more polynucleotides, expression vectors, or constructs, encoding a human IgG1, IgG2, IgG3, or IgG4, antibody. In some embodiments, the cell comprises one or more polynucleotides, expression vectors, or constructs, encoding a chimeric antibody. In some embodiments, the cell comprises one or more polynucleotides, expression vectors, or constructs, encoding a polynucleotide encoding a humanized antibody.

In some embodiments, the method further comprises extraction and/or purification of the antibody or fragment thereof. In some embodiments, the antibody or fragment thereof is extracted and/or purified from a cell supernatant or cell lysate. In some embodiments, the cell comprises one or more polynucleotides, expression vectors, or constructs, encoding a human antibody or fragment thereof. In some embodiments, the fragment comprises an antigen binding fragment. In some embodiments, the cell comprises one or more polynucleotides, expression vectors, or constructs, encoding a human IgG. In some embodiments, the cell comprises one or more polynucleotides, expression vectors, or constructs, encoding a human IgG1, IgG2, IgG3, or IgG4, antibody. In some embodiments, the cell comprises one or more polynucleotides, expression vectors, or constructs, encoding a chimeric antibody. In some embodiments, the cell comprises one or more polynucleotides, expression vectors, or constructs, encoding a polynucleotide encoding a humanized antibody. In some embodiments, one or more polynucleotides encodes the LC1 and LC2 at a ratio that is, or is about, 8:1 to 1:8, optionally 1:1, 2:1, 3:1, 4:1, 5:1, 6:1, 7:1, 8:1, 9:1, or 10:1, optionally wherein the ratio is, or is about, 1:5. In some embodiments, one or more polynucleotides encodes the HC1 and HC2 at a ratio that is, or is about, 8:1, 7:1, 6:1, 5:1, 4:1, 3:1, 2:1, 1:1, 1:2, 1:3, 1:4, 1:5, 1:6, 1:7, or 1:8, optionally 1:1, 1.5:1, or 2:1. In some embodiments, the one or more polynucleotides encodes the HC1 and LC2 at a ratio that is, or is about, 1:0.5 to 1:3, optionally 1:0.5, 1:1, 1:1.5, 1:2, 1:2.5, or 1:3. In some embodiments, the one or more polynucleotides encodes the HC1, HC2, LC1, and LC2 at a ratio that is, or is about, 1:1:1:1, 1:1:1.5:1.5, 1:1:2:1, or 2:2:5:1. In some embodiments, the one or more polynucleotides encodes an IgG Fab or antibody. In some embodiments, one or more expression vectors or constructs encode the HC1, HC2, LC1, and LC2. In some embodiments, a cell expresses the HC1, HC2, LC1, and LC2, optionally wherein the one or more polynucleotides and/or vectors encode the HC1, HC2, LC1, and LC2. In some embodiments, the cell expresses the LC1 and LC2 at a ratio that is, or is about, 8:1 to 1:8, optionally 1:1, 2:1, 3:1, 4:1, 5:1, 6:1, 7:1, 8:1, 9:1, or 10:1, optionally wherein the ratio is, or is about, 1:5. In some embodiments, the cell expresses the HC1 and HC2 at a ratio that is, or is about, 8:1, 7:1, 6:1, 5:1, 4:1, 3:1, 2:1, 1:1, 1:2, 1:3, 1:4, 1:5, 1:6, 1:7, or 1:8, optionally 1:1, 1.5:1, or 2:1. In some embodiments, the cell expresses the HC1 and LC2 at a ratio that is, or is about, 1:0.5 to 1:3, optionally 1:0.5, 1:1, 1:1.5, 1:2, 1:2.5, or 1:3. In some embodiments, the cell expresses the HC1, HC2, LC1, and LC2 at a ratio that is, or is about, 1:1:1:1, 1:1:1.5:1.5, 1:1:2:1, or 2:2:5:1. In some embodiments, the cell expresses an IgG Fab or antibody. In some embodiments, the cell is a mammalian cell or bacterial cell. In some embodiments, the cell is a HEK293 cell, Chinese hamster ovary (CHO) cell, or a NS0 cell. In some embodiments, the method comprises extraction and/or purification of the expressed antibody or fragment thereof. In some embodiments, the expressed antibody or fragment thereof is extracted and/or purified from a cell supernatant or cell lysate.

### EXAMPLES

### Example 1

The Fab Structure in PDB: 7REWwas visualized using the open-source software Pymol. The interaction interface between the CL and CH1 in 7REW was identified, and residues in the CL and CH1 domains contributing the most to the Fab domain packing stabilities were identified as L135 (CL), F170 (CH1), S183 (CH1), and V185 (CH1), based on EU numbering (L135 in LC and F176, S189, and V191 in HC, based on AA sequence numbering in 7REW).

The design criteria for specific LC-HC pairing were as follows: 1) Mutant LC and LC should be packed with similarly as wild type LC and HC interactions at CL and CH1 domains; and 2) Mutant HC-wt LC interactions and mutant LC-wt HC interactions should be unfavored (steric clashes of side chains). A group of mutation sets was assembled at L135 (CL) and F170, S183, V185 (CH1), based on EU numbering (based on AA sequence numbering in 7REW, the numberings are L135 in LC and F176, S189, and V191 in HC), and the final mutation sets according to AA sequence numbering in 7REW that fit the design criteria were summarized in Table 1.

TABLE 1

| Mutation | | | | | | Engineered |
|---|---|---|---|---|---|---|
| set | HC | LC | Engineered Site in HC | | | Site in LC |
| Starting | wt | wt | H176 | H198 | H191 | L135 |
| wt | wt | wt | F176F | S198S | V191V | L135L |
| JT1/F | JT1 | F | F176L | S198I | V191V | L135F |
| JT2/F | JT2 | F | F176I | S198I | V191V | L135F |
| JT3/F | JT3 | F | F176I | S198V | V191I | L135F |
| JT4/F | JT4 | F | F176L | S198I | V191L | L135F |
| JT5/F | JT5 | F | F176I | S198V | V191L | L135F |
| JT4/I | JT4 | I | F176L | S198I | V191L | L135I |
| JT7/F | JT7 | F | F176I | S198L | V191L | L135F |
| JT3/I | JT3 | I | F176I | S198V | V191I | L135I |
| JT8/F | JT8 | F | F176L | S198L | V191V | L135F |
| JT9/I | JT9 | F | F176L | S198V | V191L | L135I |
| JT10/F | JT10 | F | F176V | S198I | V191I | L135F |
| JT11/F | JT11 | F | F176V | S198I | V191L | L135F |
| JT4/V | JT4 | V | F176L | S198I | V191L | L135V |
| JT12/F | JT12 | F | F176I | S198L | V191V | L135F |
| JT13/F | JT13 | F | F176I | S198L | V191V | L135F |
| JT14/F | JT14 | V | F176F | S198V | V191I | L135V |

These mutations were introduced into the CL and CH1 domains of the antibody whose structure is represented by PDB 7REW.

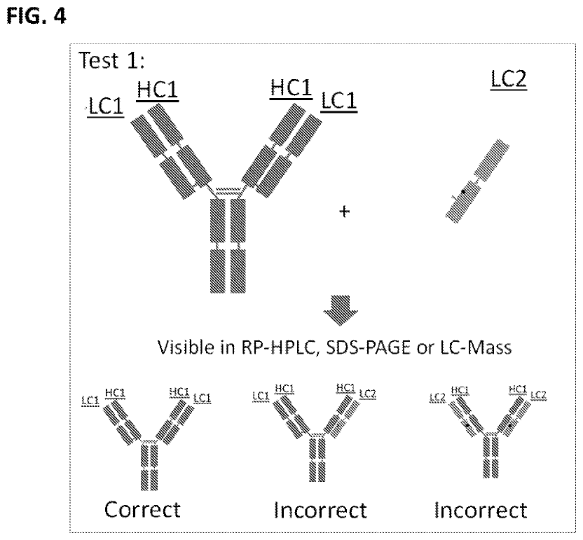

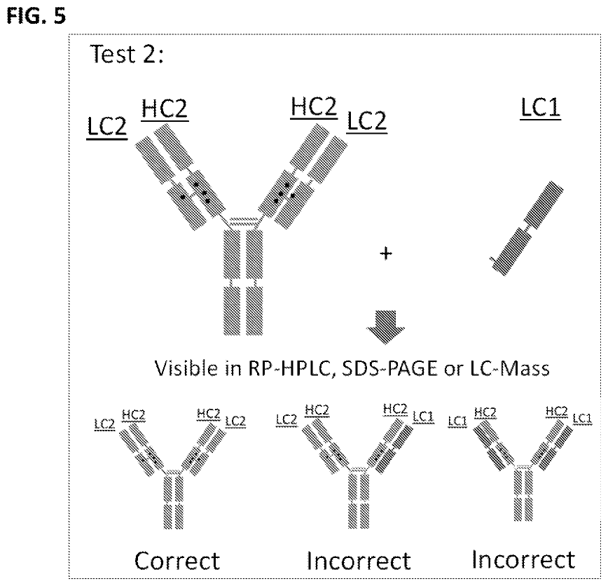

FIG. 4 is an illustrative representation of an embodiment of pairing analysis of WT heavy chain and WT light chain in the presence of mutant light chain with L135F.

Expression plasmids encoding the 7REW antibody's LC and HC were coexpressed in HEK293 cells through transient transfections in the presence of the mutant LC containing E124C, L135F and C214S. The packing energy calculations in Table 1 suggested that the mutant LC containing L135F would not pair with wt HC, resulting in incorrect wildtype LC and HC pairing. The assembly of intact correctly paired LC and HC was analyzed using SDS-PAGE under non-reducing conditions or Reverse Phase HPLC on a Biphenyl column using water/acetonitrile (0.1% TFA) as the mobile phase. Incorrectly paired HC and mutant LC (L135F, C214S) appeared as IgG missing LC or LC(s) under SDS-PAGE or RPHPLC, as these analyses were performed under denaturing conditions that can separate subunits if they are not covalently linked.

FIG. 5 is an illustrative representation of an embodiment of pairing analysis of mutated heavy chains pairing with a mutated in the presence of WT light chain.

Expression plasmid pairs encoding LC and HC with mutation sets listed in Table 1 plus additional LC mutations of E124C and C214S (lambda) and HC mutations of F126C and C220S and WT LC were co-expressed in HEK293 cells through transient transfections. LC mutations of E124C and C214S and HC mutations of F126C and C220S provided a readout of mutant LC/HC pairing through disulfide bridge formation between LC's E124C and HC's F126C. Mis-paired wt LC and mutant HC did not have disulfide bond linkage because of the C2220S mutations in the mutant HC. Correct assembly of LC and HC was analyzed using SDS-PAGE under non-reducing conditions or Reverse Phase HPLC on a Biphenyl column using water/acetonitrile (0.1% TFA) as the mobile phase. Mutant HC incorrectly paired with wt LC appeared as an IgG missing LC or LC(s) under SDS-PAGE or RP-HPLC, as these analyses were performed under denaturing conditions that can separate subunits if they are not covalently linked.

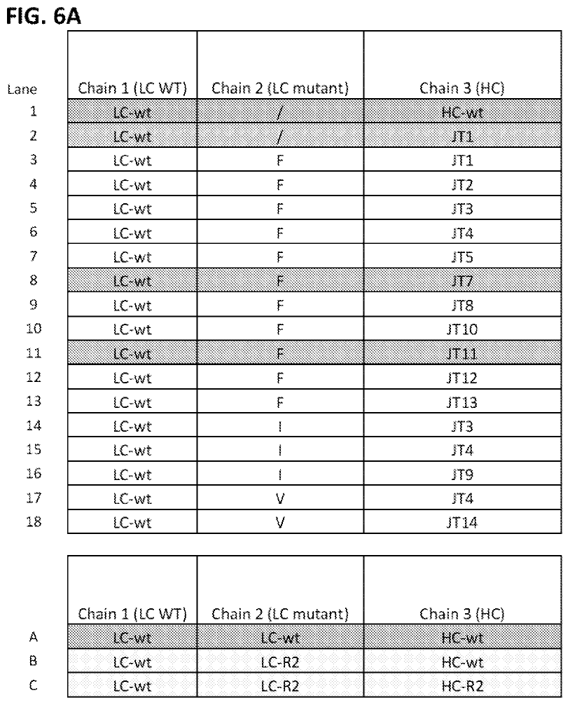

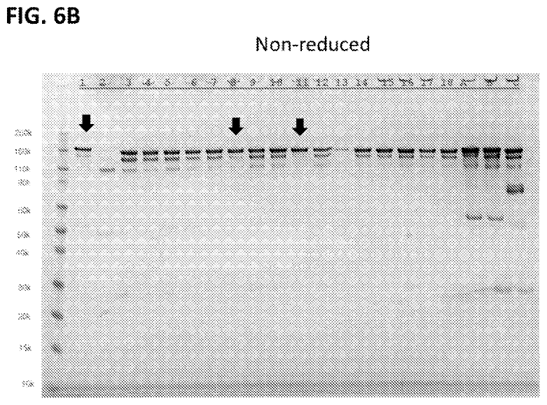

FIG. 6A is a table indicating the identity of an embodiment of samples in each lane of the SDS-page gel in FIG. 6B.

FIG. 6B is a SDS-PAGE gel of an embodiment of different HC/LC pairings under non-reducing conditions. Mutation sets that enhance LC/HC pairing specificity will enhance the percentage of correctly assembled intact antibodies (upper band of two LCs and two HCs) in the presence of similar amount of competing unchanged wt LC-HC-JT7 or JT11 paired with LC-F achieved comparable wt IgG's correct assembly in the presence of the competing wt LC.

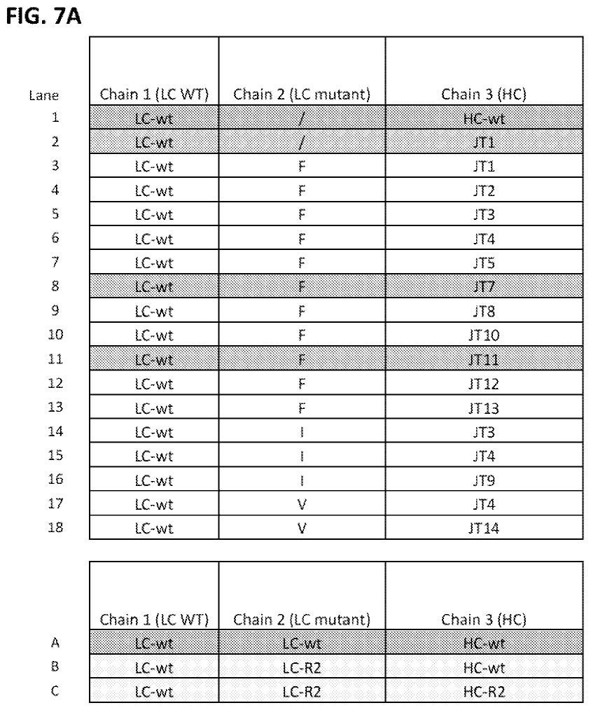

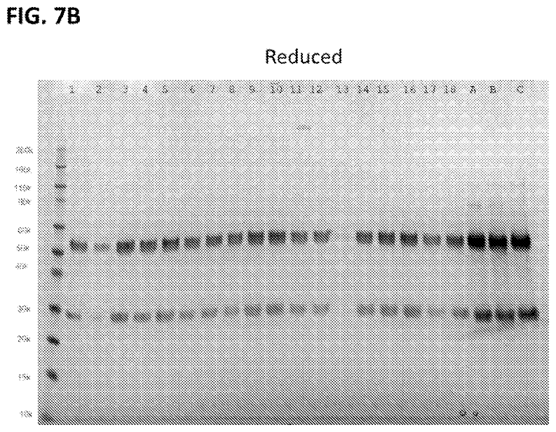

FIG. 7A is a table indicating the identity of an embodiment of samples in each lane of the SDS-page gel in FIG. 7B.

FIG. 7B is an SDS-page gel of an embodiment of different HC/LC pairings under reducing conditions.

Expression plasmid pairs encoding LC and HC with mutation sets listed in Table 1 plus additional LC mutations of E124C and C2124S and HC mutations of F126C and C220S and wt LC were co-expressed in 293 cells through transient transfections as in FIG. 5. IgG molecules in the conditioned were analyzed using a Protein A purification analysis for titer and LC-HC pairings.

During transient expression of the IgG molecules, wt LC and mutant LC were co-transfected at an equal amount to provide a readout of mutant LC/HC pairing specificity: when mutant LC pairs to mutant HC, IgG molecules were correctly assembled and migrated on SDS-PAGE as intact IgG band under non-reducing conditions. Mis-paired wt LC and mutant HC did not form disulfide bonds, leading to the detection of IgG bands missing LCs under non-reducing conditions (lower bands). The mutation sets listed in Table 1 are represented in lanes 1-18. Through this analysis, two mutation sets (F/JT7 and F/JT11) that demonstrated the desired pairing specificity were identified.

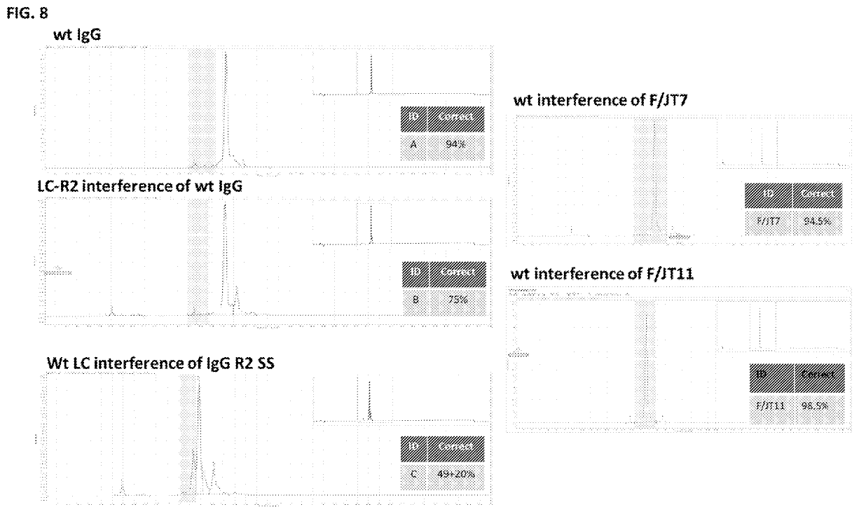

FIG. 8 is a series of chromatograms of an embodiment of quantification by RP-HPLC of correct LC-HC pairing in positive controls and selected mutation sets.

As can be seen in FIG. 8, proper LC-HC pairing in F/JT7 and F/JT11 mutants occurs at least as often as in a wt IgG, in the presence of interfering wt LC.

Quantification of proper LC-HC pairing is shown in Table 2.

TABLE 2

| | Chain 1 | Chain 2 | | | % of |
|---|---|---|---|---|---|
| Sample | (LC | (LC | Chain 3 | Titer | correct |
| ID | WT) | mutant) | (HC) | (mg/L) | pairing |
| 1 | LC-wt | / | HC-wt | 81 | 94 |
| 2 | LC-wt | / | JT1 | 48 | 9.2 |
| 3 | LC-wt | F | JT1 | 138 | 79.9 |
| 4 | LC-wt | F | JT2 | 143 | 83.3 |
| 5 | LC-wt | F | JT3 | 118 | 80.4 |
| 6 | LC-wt | F | JT4 | 108 | 83.7 |
| 7 | LC-wt | F | JT5 | 116 | 87.4 |
| 8 | LC-wt | F | JT7 | 95 | 94.5 |
| 9 | LC-wt | F | JT8 | 133 | 76 |
| 10 | LC-wt | F | JT10 | 126 | 86 |
| 11 | LC-wt | F | JT11 | 98 | 98.5 |
| 12 | LC-wt | F | JT12 | 112 | 73.6 |
| 13 | LC-wt | F | JT13 | 0 | 82.4 |
| 14 | LC-wt | I | JT3 | 130 | 90.6 |
| 15 | LC-wt | I | JT4 | 138 | 88.1 |
| 16 | LC-wt | I | JT9 | 156 | 88.1 |
| 17 | LC-wt | V | JT4 | 124 | 90.5 |
| 18 | LC-wt | V | JT14 | 120 | 90.1 |
| A | LC-wt | LC-wt | HC-wt | 200 | 94 |
| B | LC-wt | LC-R2 | HC-wt | 156 | 75 |
| C | LC-wt | LC-R2 | HC-R2 | 124 | 49 + 20 |

As is clear from Table 2, proper LC-HC pairing in F/JT7 and F/JT11 mutants occurs at least as often as in a wt IgG.

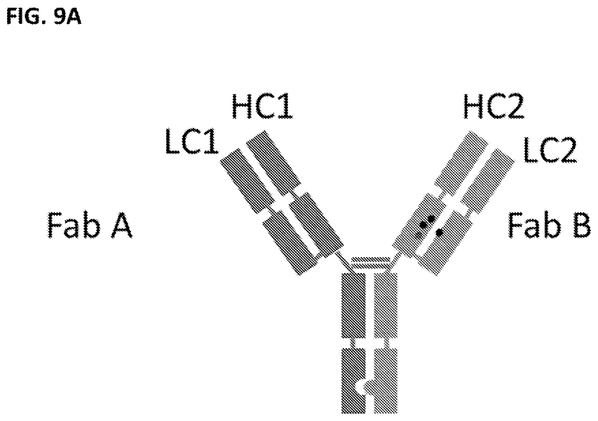

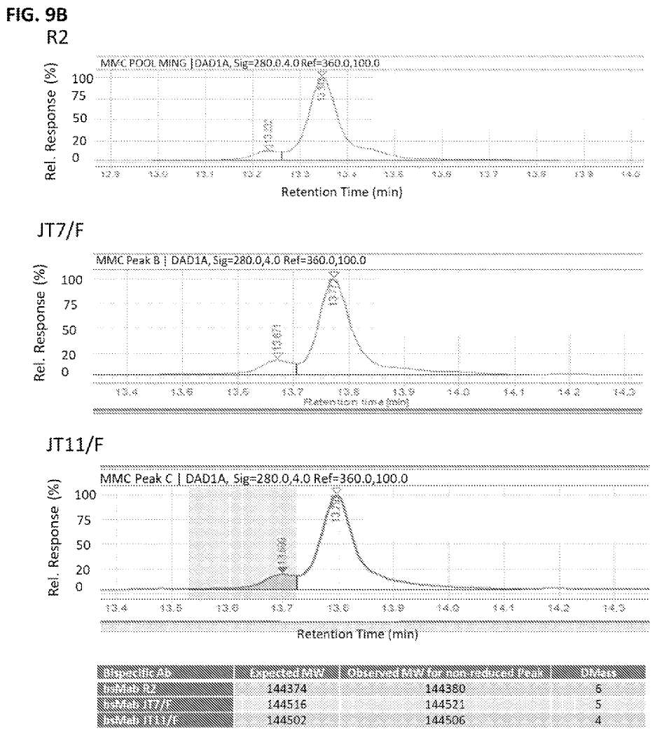

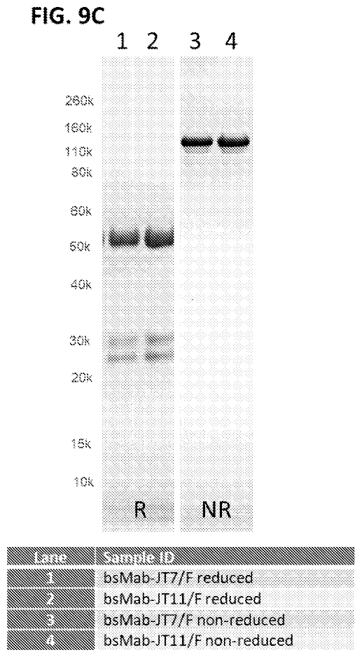

FIG. 9A is an illustrative representation of an embodiment of correct pairing of heavy and light chains of JT7/F and JT11/F dimerization pairs in a bispecific antibody.

FIG. 9B is a chromatogram of an embodiment of purity and correct assembly analyses by RP-HPLC on purified bispecific antibodies using LC/HC pairing strategies of alternative disulfide (R2), JT7/F and JT11/11 dimerization pairs.

FIG. 9C is an SDS-page gel of an embodiment of purified bispecific antibodies based on JT7/F and JT11/F dimerization mutation sets under reducing and nonreducing conditions.

BISPECIFIC MAB-R2: plasmids encoding Fab 1 from LC1-wt kappa LC; HC1-wt HC with hole mutation (T366S, L368A, Y407V, EU numbering); and Fab 2 from LC2-lambda LC with E124C, C214S (EU numbering); HC2-(F126C, C220S) with knob mutation (S366W, EU numbering) were co-transfected transiently in HEK 293 cells. The individual subunits were assembled in HEK 293 cells as BISPECIFIC MAB--R2 bispecific antibodies and related misassembled impurities. BISPECIFIC MAB-R2 was used as a reference benchmark bispecific antibody whose LC-HC specific pairings are conveyed by an alternative disulfide bond between LC's CL domain and HC's CH1 domain (E124C in LC linked to F126C) according to U.S. Pat. No. 10,344,099 B2.

BISPECIFIC MAB--JT7: plasmids encoding Fab 1 from LC1-wt kappa LC; HC1-wt HC with hole mutation (T366S, L368A, Y407V, EU numbering); and Fab 2 from LC2-lambda LC with L135F (EU numbering); HC2-(JT7 mutation set) with knob mutation (S366W, EU numbering) were co-transfected transiently in HEK 293 cells. The individual subunits were assembled in HEK 293 cells as BISPECIFIC MAB--JT7 bispecific antibodies and related misassembled impurities.

BISPECIFIC MAB--JT11: plasmids encoding Fab 1 from LC1-wt kappa LC; HC1-wt HC with hole mutation (T366S, L368A, Y407V, EU numbering); and Fab 2 from LC2-lambda LC with L135F (EU numbering); HC2-(JT11 mutation set) with knob mutation (S366W, EU numbering) were co-transfected transiently in HEK 293 cells. The individual subunits are assembled in HEK 293 cells as BISPECIFIC MAB-JT7 bispecific antibodies and related misassembled impurities.

BISPECIFIC MAB--R2, BISPECIFIC MAB--JT7 and BISPECIFIC MAB-JT 11 in the conditioned media from transfected 293 cells were captured by a Mabselect PrismA (Protein A column). Transfection host derived impurities were removed by Phosphate Buffer Saline wash. Bound IgG were recovered by acidic elution using 87 mM Acetate, pH 3.5. Fractions containing antibodies were pooled and its pH adjusted to pH5.0. These pooled fractions are referred to as ProA pool. The ProA pools for BISPECIFIC MAB--R2, BISPECIFIC MAB--JT7 and BISPECIFIC MAB--JT11 were further purified on a multimodal chromatography column of Capto-MMC ImpRes (Cytiva) using a pH gradient elution from pH 5.0 to pH8.9. Capto-MMC fractions were analyzed on SDS-PAGE and correctly assembled bispecific antibody fractions were combined and analyzed for biospecific activity and molecule assembly.

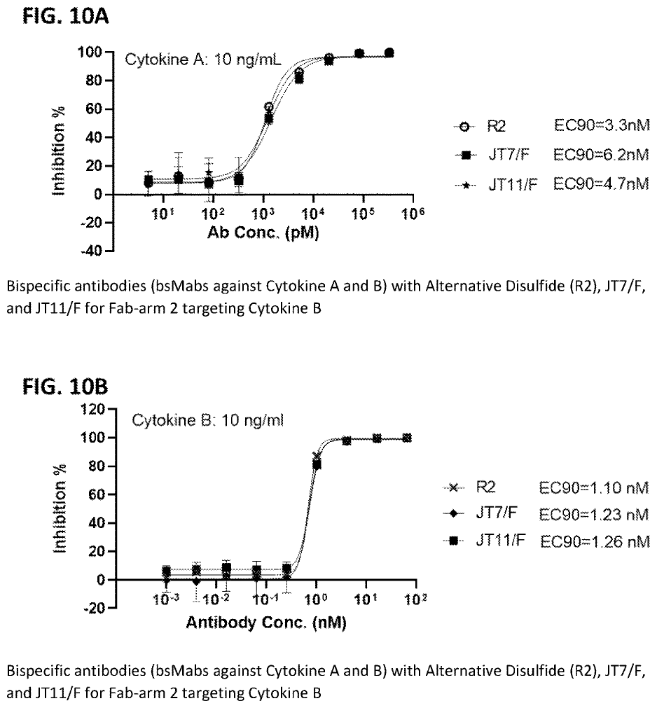

FIG. 10A is a graph of an embodiment of the neutralizing activity of bispecific molecules' Fab arm 1 in a HEK-293 reporter cells.

Human cytokine A blockage assay by bispecific antibodies in HEK-293 reporter cells. Binding of cytokine A to its receptor complex activates downstream signaling pathway, resulting a cytokine A induced expression of luciferase in the HEK-293 reporter cells. To analyze the cytokine neutralizing activity, the reporter HEK-293 cells were simultaneously incubated with 10 ng/ml human cytokine A and bispecific antibodies at 37° C. for 5 hr. After 5-hour co-incubation, bioluminescence signal was measured. The data are presented using inhibition percentage of cytokine-luciferase activities.

FIG. 10B is a graph of an embodiment of the neutralizing activity of bispecific molecules' Fab arm 2 in a cytokine B blockage assay by bispecific antibodies in a HEK-293 reporter cells.

Human cytokine B blockage assay by bispecific antibodies in HEK-293 reporter cells. Binding of cytokine B to its receptor complex activates downstream signaling pathways, resulting in expression of alkaline phosphatase. Here, the 293-cell line containing the cytokine B receptor complex on the cell surface was stably transfected with Secreted Embryonic Alkaline Phosphatase (SEAP) under the control of cytokine B signal pathways. HEK-293 reporter cell line was used to monitor the cytokine B's signaling pathway by measuring SEAP activity in conditioned media. In the cytokine B neutralization assay, the HEK-293 reporter cells were simultaneously incubated with 10 ng/mL human cytokine B and bispecific antibodies at 37° C. for 5 hours. After 5-hour co-incubation, bioluminescence signal was measured. The data are presented using inhibition percentage of SEAP activities.

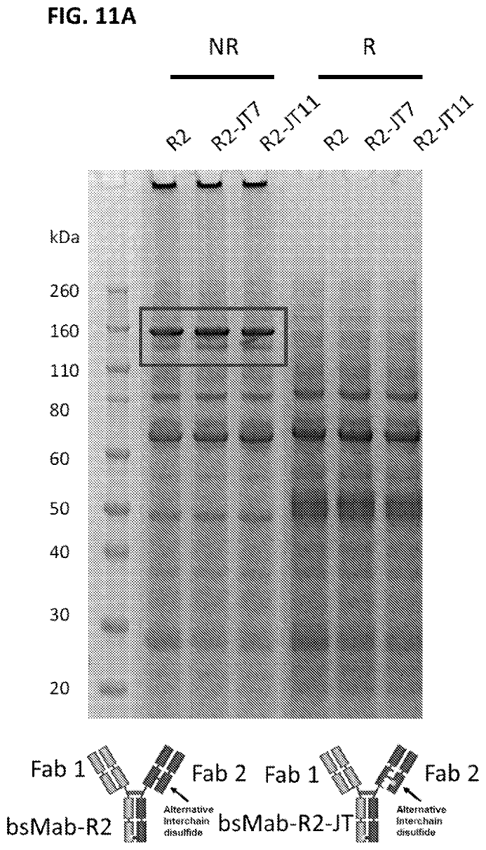

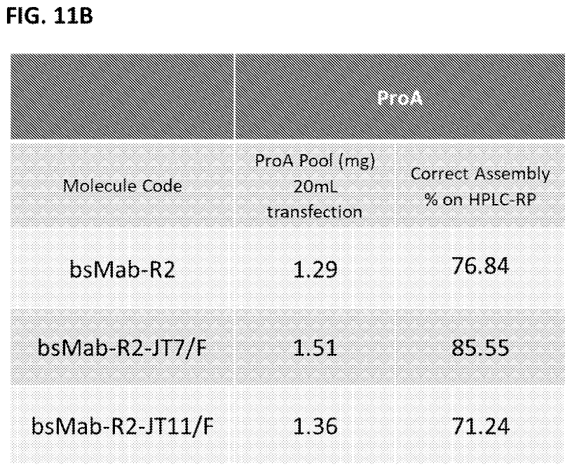

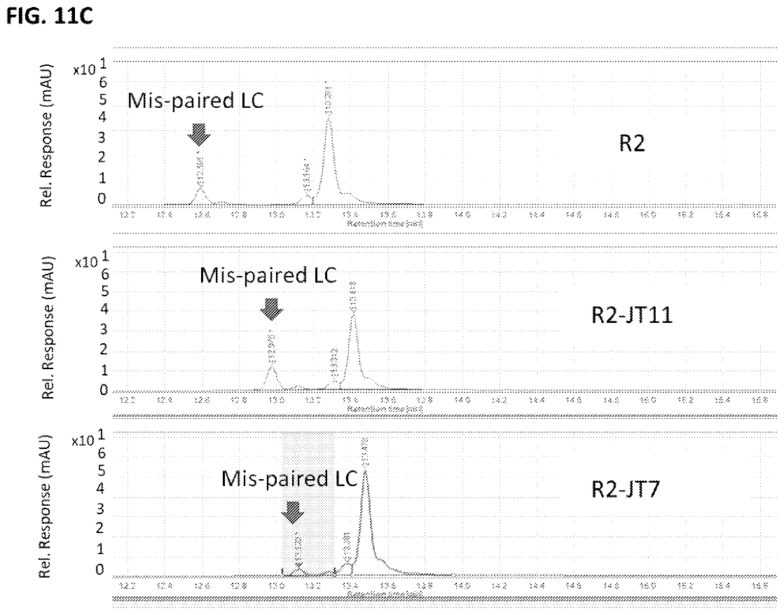

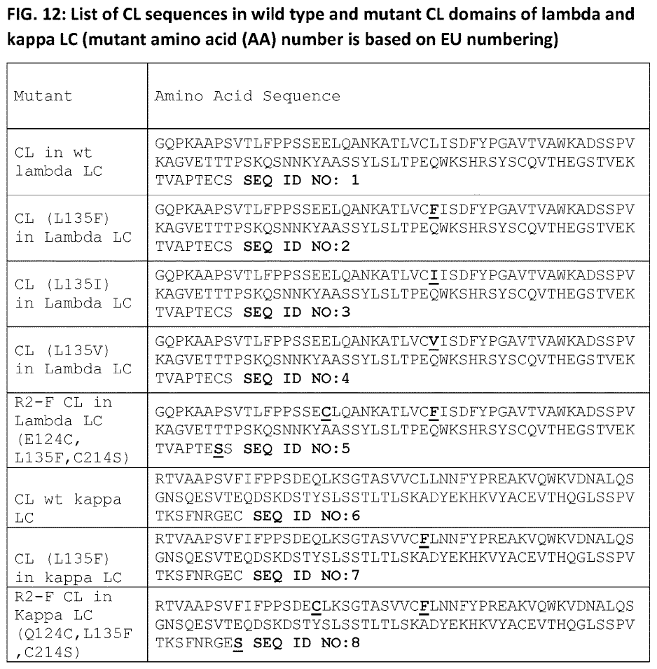

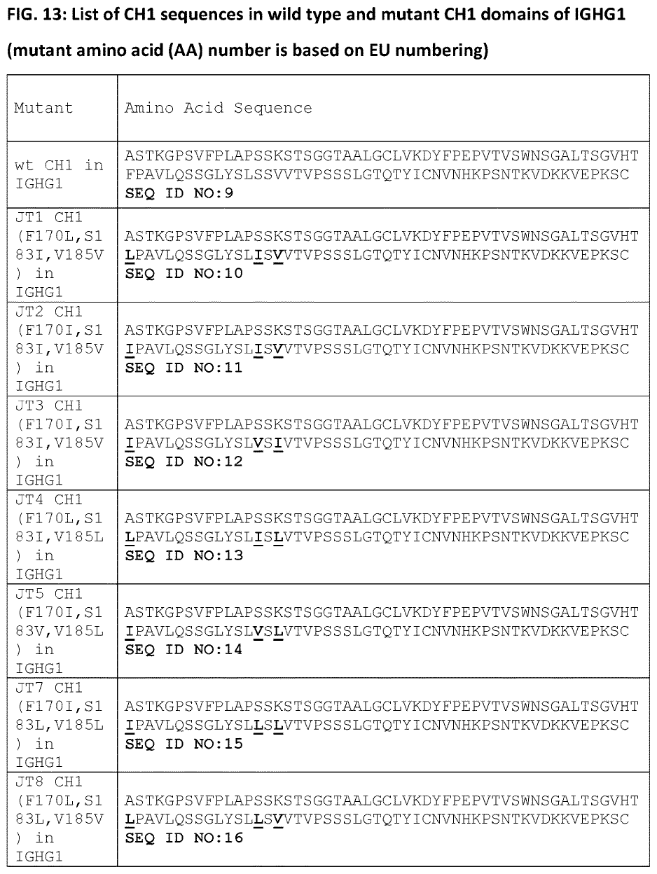

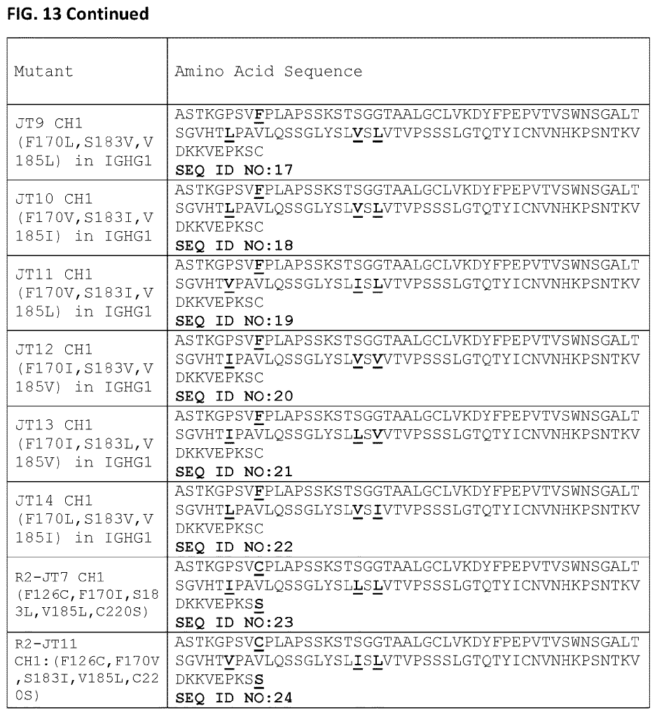

FIG. 11 shows combination of mutation sets JT11/F or JT7/F with alternative disulfide R2 benefits molecule assembly of the bispecific antibodies and their expression yields.

FIG. 11A is an SDS-PAGE analysis under reducing and non-reducing conditions for an embodiment of different conditioned media from HEK-293 cells that have been transfected with different plasmids encoding the HCs and LCs of bispecific antibodies.

FIG. 11B is a table of quantification of an embodiment of the yield for expression and capturing of bispecific antibodies.

FIG. 11C is a set of chromatograms of an embodiment of quantification by RP-HPLC of correct LC-HC pairing of plasmid produced JT7 and JT11 dimerization pairs.

BISPECIFIC MAB--R2: plasmids encoding Fab 1 from LC1-wt kappa LC; HC1-wt HC with hole mutation (T366S, L368A, Y407V, EU numbering); and Fab 2 from LC2-lambda LC with E124C, C214S (EU numbering); HC2-(F126C, C220S) with knob mutation (S366W, EU numbering)—a reference benchmark bispecific antibody based on technology from U.S. Pat. No. 10,344,099 B2.

BISPECIFIC MAB--R2-JT7: plasmids encoding Fab 1 from LC1-wt kappa LC; HC1-wt HC with hole mutation (T366S, L368A, Y407V, EU numbering); and Fab 2 from LC2-lambda LC with E124C and L135F, C214S (EU numbering); HC2-(F126C,F170I,S183L, V185L,C220S) with knob mutation (S366W, EU numbering).

ISPECIFIC MAB--R2-JT11: plasmids encoding Fab 1 from LC1-wt kappa LC; HC1-wt HC with hole mutation (T366S, L368A, Y407V, EU numbering); and Fab 2 from LC2-lambda LC with E124C and L135F, C214S (EU numbering); HC2-(F126C,F170V,S183I, V185L, C220S) with knob mutation (S366W, EU numbering).

BISPECIFIC MAB--R2, BISPECIFIC MAB--R2-JT7 and BISPECIFIC MAB--R2-JT11 were transfected into 293 cells. These bispecific antibodies in the conditioned media from transfected 293 cells were captured by a Mabselect PrismA (Protein A column). ProA pools were analyzed for correct bispecific assembly by SDS-PAGE and RP-HPLC. The correctly assembled bsMab percentage is estimated from the intact bsMab peaks on RP-HPLC (FIG. 11C).

Mutation sets F/JT7 (L135 in CL and F1701, S183L, and V185L in CH1 EU Numbering) and F/JT11 (L135 in CL and F170V, S183I, and V185L EU Numbering) that enhanced LC/HC pairing specificities in antibodies with lambda LC (FIG. 6 as an example) were introduced into the Fab structure (the Fab in PDB 1A4J) with a kappa LC as the light chain. The corresponding mutation-sets in the kappa Fab (PDB 1A4J), compared to that of the wild-type Fab, is summarized in Table 3.

TABLE 3

| Mutation | | | | | | Engineered | |
|---|---|---|---|---|---|---|---|
| set | HC | LC | Engineered Site in HC | | | Site in LC | |
| Starting | wt | wt | H170 | H183 | H185 | L135 | EU |
| | | | | | | | numbering |
| | | | F172F | S185S | V187V | L140L | number |
| | | | | | | | in 1A4J |
| wt | wt | wt | F172F | S185S | V187V | L140L | |
| JT7/F | JT7 | F | F172I | S185L | V187L | L140F | |
| JT11F | JT11 | F | F172V | S185I | V187L | L140F | |

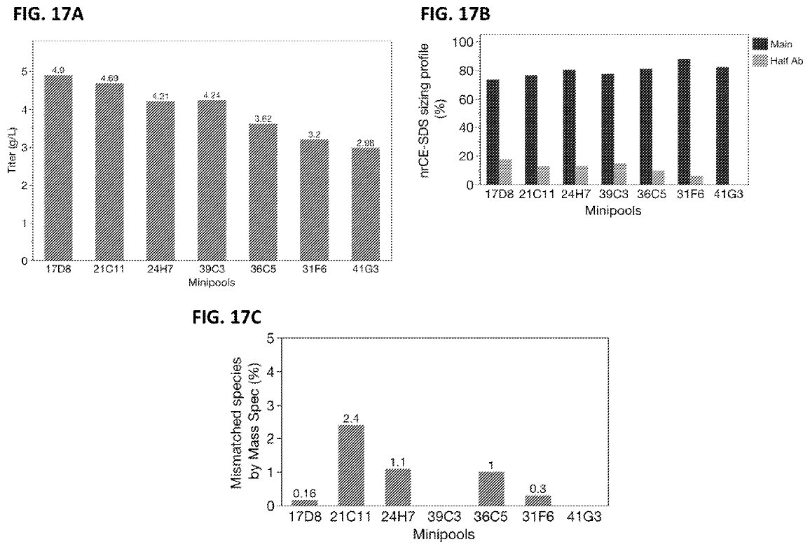

FIG. 17A is a bar graph depicting quantification of bispecific antibody expression in CHO cells.

FIG. 17B is a bar graph depicting quantification of ProA pools analyzed by capillary electrophoresis sodium dodecyl sulfate (CE-SDS).

FIG. 17C is a bar graph depicting an embodiment of quantification of mismatched species by Mass Spec.

CHO cells transfected with DNA encoding 2 HCs and 2 LCs of bispecific antibody for Cytokine A and Cytokine B were plated as minipools of 20,000 cells/well in 96-well plates in MSX selection medium. Minipools that stably express the bispecific molecule were able to recover after 2 weeks of MSX selection and were screened for bispecific antibody titer. The top 8 minipools were subjected to a 14-day cell culture production assay. As can be seen in FIG. 17A, titers of the top 8 minipools ranged from 2.98 to 4.9 g/L.

Conditioned media from top 8 CHO minipools of the bispecific antibody for Cytokine A and Cytokine B were captured on a Protein A resin-MabSelect SuRe. Non-specific bindings were washed off with PBS (phosphate buffered saline) solution. Expressed antibodies were eluted with 0.5% acetic acid, pH 3.5 and neutralized with 15% volumes of 1 M Tris-HC1, pH 8.0 to a final pH of 7.4. The neutralized Protein A eluents are referred to as ProA pools.

ProA pools from the top 8 minipools were analyzed by capillary electrophoresis sodium dodecyl sulfate (CE-SDS). Main peak: the expressed antibody recovered as the intact antibody species. Half antibody was correctly paired half antibody for Cytokine B (Fab for cytokine B on Fc without correctly formed hinge disulfide).

ProA pools were digested with FabALACTICA (from Genovis) to cut the Fabs off from the Fc fragments. Fabs were analyzed by LC-MS for correcting pairing percentage (FIG. 17C). Mispaired (mismatched) species were calculated as (X+Y)/Total Fab in percentage. X=HC_Cytokine A paired to LC_Cytokine B; Y=HC_Cytokine B paired to LC_Cytokine A; Total Fab=correct Fab_Cytokine_A+correct Fab_Cytokine B+X+Y.

### Example 2

To ensure LC-HC pairing fidelity in bispecific IgG1 molecules, especially when both Fabs are with kappa LCs as light chains, pairing mutations were introduced to VL-CL interface in addition to the CL-CH1 pairing mutations F/JT7 or F/JT11. Residue pairs that are on VL-VH interface were mutated to Cys for disulfide bond formation, or to opposite charge residues for electrostatic interactions (Table 4).

TABLE 4
LC and HC pairing mutations for BsIgG1 targeting Cytokine C and D.

| | Cytokine C Fab (First Fab) | | | | Cytokine D Fab (Second Fab) | | | |
|---|---|---|---|---|---|---|---|---|
| | VH | CH1 | VL | CL | VH | CH1 | VL | CL |
| V1 | Q39K | WT | Q38D | WT | Q39D | F170V/S183I/V185L | Q38K | L135F |
| V2 | Q39D | WT | Q38K | WT | Q39K | F170V/S183I/V185L | Q38D | L135F |
| V3 | G44K | WT | G100D | WT | G44D | F170V/S183I/V185L | G100K | L135F |
| V4 | G44D | WT | G100K | WT | G44K | F170V/S183I/V185L | G100D | L135F |
| V5 | G44C | WT | G100C | WT | WT | F170V/S183I/V185L | WT | L135F |
| V6 | WT | WT | WT | WT | G44C | F170V/S183I/V185L | G100C | L135F |
| V7 | G44K | WT | G101D | WT | G44D | F170V/S183I/V185L | G101K | L135F |
| V8 | G44D | WT | G101K | WT | G44K | F170V/S183I/V185L | G101D | L135F |

Pairing for the HCs in the bispecific IgGs were conveyed the knob mutation (T366W) in the HC of Cytokine D and the hole mutations (T366S/S368A/Y407V) in the HC of Cytokine C.

To screen mutations that improve pairing specificity, Cytokine C and Cytokine D HC and k-LC variants (Table 5) were co-transfected at a plasmid ratio for the four chains (HC1: HC2: LC1: LC2) of 1:1:1.5:1.5 into Expi293 cells. The expressed IgG molecules in conditioned media were captured on a Protein A resin-MabSelect SuRe. After Non-specific bindings were washed off with PBS (phosphate buffered saline) solution, the expressed antibodies were eluted with 0.5% acetic acid, pH 3.5 and neutralized with 15% volumes of 1 M Tris-HC1, pH 8.0 to a final pH of 7.4. Purified IgG molecules from the conditioned media were then analyzed by liquid chromatography-mass spectrometry (LC-MS).

The pools were further polished on a mixed modal column Capto MMC ImpRes. Fractions were pooled separately as bispecific antibody and mispaired pools and analyzed on LC-MS.

Correct bispecific antibodies and mis assembled IgGs from ProA pools and MMC pools were first treated with PNGase to generate deglycosylated intact proteins. Deglycosylated intact molecules were analyzed by LC-MS to reveal the mispairing impurities. The percentage of correctly assembled BsIgG1 was calculated by quantifying all IgG1 species present (excluding half antibodies and homodimers of half antibodies (Table 5)). IgG molecules with the LC pairing to both HCs are labeled as common Cytokine D LC or common Cytokine C LC mispairing impurifies. ProA and MMC pools of IgG molecules were also treated with FabALACTICA (Genovis) to release Fab fragments from IgG molecules. The percentage of mispairing in each Fab containing arm was also determined by LC-MS (Table 6). Both Table 5 and Table 6 indicate that the second purification step, Capto MMC ImpRes column, did not successfully remove mispairing contaminants when Cytokine D's LC paired to both the HCs of Cytokine D and Cytokine C.

TABLE 5
Percentage of BsIgG1 and mispairing contaminants in ProA
and Capto MMC pools analyzed by intact LC-MS

| | | | Common | Common |
|---|---|---|---|---|
| | | correct | Cytokine D | Cytokine C |
| | | BsIgG1 | (Second Fab) | (First Fab) |
| | | (%) | LC (%) | LC (%) |
| proA | V1 | 61.3 | 6.8 | 31.9 |
| | V2 | 70.6 | 13.2 | 16.3 |
| | V5 | 100.0 | 0.0 | 0.0 |
| | V6 | 92.7 | 7.3 | 0.0 |
| | V7 | 52.6 | 11.5 | 36.0 |
| MMC | V1 | 87.8 | 12.2 | 0.0 |
| | V2 | 85.1 | 14.9 | 0.0 |
| | V5 | 100.0 | 0.0 | 0.0 |
| | V6 | 93.5 | 6.5 | 0.0 |
| | V7 | 81.7 | 18.3 | 0.0 |

TABLE 6
Percentage of correct and mispairing Fab in ProA and Capto MMC pools determined
by Fab fragment LC-MS analysis.

| | | Cytokine C Fab (First Fab) | | Cytokine D Fab (Second Fab) | |
|---|---|---|---|---|---|
| | | Correct Fab | Mis-paired Fab | Correct Fab | Mis-paired Fab |
| | | (Cytokine C | (Cytokine C HC/ | (Cytokine D | (Cytokine D HC/ |
| | | HC/LC) | Cytokine D LC) | HC/LC) | Cytokine C |
| | | (%) | (%) | (%) | LC) (%) |
| proA | V1 | 85.8 | 14.2 | 70.0 | 30.0 |
| | V2 | 88.1 | 11.9 | 88.6 | 11.4 |
| | V5 | 100.0 | 0.0 | 100.0 | 0.0 |
| | V6 | 95.3 | 4.7 | 100.0 | 0.0 |
| | V7 | 85.5 | 14.5 | 64.9 | 35.1 |
| MMC | V1 | 81.5 | 18.5 | 100.0 | 0.0 |
| | V2 | 85.5 | 14.5 | 100.0 | 0.0 |
| | V5 | 100.0 | 0.0 | 100.0 | 0.0 |
| | V6 | 89.4 | 10.6 | 100.0 | 0.0 |
| | V7 | 75.6 | 24.4 | 100.0 | 0.0 |

To demonstrate the robustness of the pairing mutations in bispecific IgG's assembly and production process, HEK293 cells were transfected with different amount of cytokine C's LC-C and cytokine D's LC-D at 1:1, 2:1 and 5:1 respectively for V2 constructs. Cytokine C HC-C and cytokine D HC-D were present in equal amounts, and the ratio of HC-C, HC-D, LC-C, LC-D was 1:1:1.5:1.5, 1:1:2:1, and 1:1:2.5:0.5. IgGs in the conditional media were first captured on and eluted from Protein A resin MabSelect SuRe., followed by a second step purification on Capto MMC ImpRes column. The ProA or MMC purified protein pools were treated with FabALACTICA (Genovis) to release the Fab fragments, which were analyzed on LC-MS to determine the percentages of HC/LC mispairing (Table 7).

TABLE 7
Percentage of correct and mispairing V2 Fabs in ProA and Capto MMC pools determined by
Fab fragment LC-MS analysis.

| | | Cytokine C Fab | | Cytokine D Fab | |
|---|---|---|---|---|---|
| | | Correct Fab | Mis-paired Fab | Correct Fab | Mis-paired Fab |
| | LC-C:LC-D | (HC-C/LC-C) | (HC-C/LC-D) | (HC-D/LC-D) | (HC-D/LC-C) |
| Pool | DNA ratio | (%) | (%) | (%) | (%) |
| ProA | 1:1 | 88.1 | 11.9 | 88.6 | 11.4 |
| | 2:1 | 95.3 | 4.7 | 78.7 | 21.3 |
| | 5:1 | 99.7 | 0.3 | 61.3 | 38.7 |
| MMC | 1:1 | 85.5 | 14.5 | 100.0 | 0.0 |
| | 2:1 | 94.1 | 5.9 | 100.0 | 0.0 |
| | 5:1 | 97.3 | 2.7 | 100.0 | 0.0 |

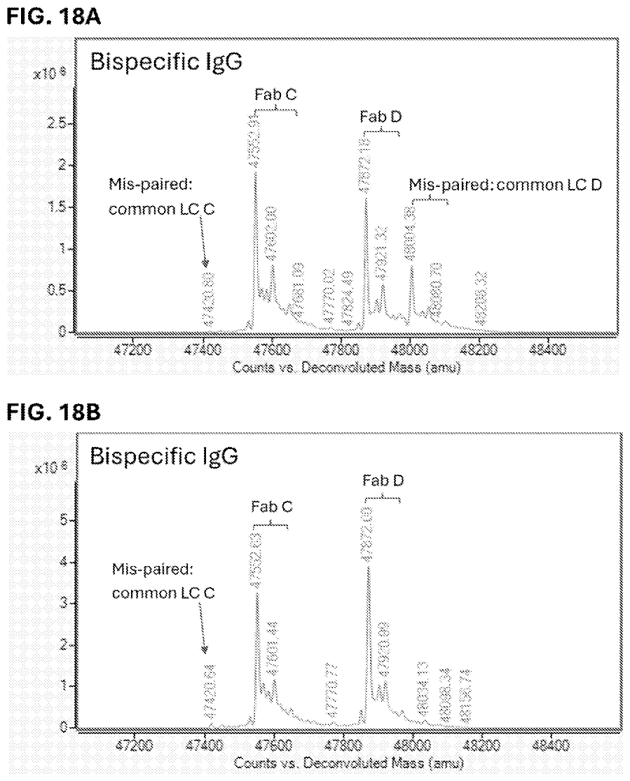

FIG. 18A is a chromatogram depicting an embodiment of quantification of Fab fragment parings as analyzed by LC-MS.

FIG. 18B is a chromatogram depicting an embodiment of quantification of Fab fragment pairings following polishing as analyzed by LC-MS.

The results indicate that increasing LC-C expression reduced common LC-D mispairing while increased common LC-C mispairing. With the second step purification removing the LC-C mispairing contaminants, the percentage of correct assembled BsIgGs in the final purified product increased.

1.-266. (canceled)

267. An antigen-binding fragment (Fab) comprising:
a heavy chain (HC) variable domain (VH), a heavy chain (HC) constant domain (CH1) of a human IgG, a light chain (LC) variable domain (VL), and a light chain (LC) constant domain (CL) of a human IgG, wherein the HC CH1 comprises a substitution at F170, S183, and V185 (EU Numbering), and the LC CL comprises a substitution at L135 (EU Numbering), wherein the HC1 does not include any substitution selected from L128F, A141M, A141T, A141I, F170M, F170Y, F170S, F170A, S181I, S181T, S181M, S183A, S183V, and V185A (EU Numbering),
and wherein the CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (European Numbering).

268. The Fab of claim 267, wherein the CH1 consists of substitutions at F170, S183 and V185 (EU Numbering), and wherein the CL consists of a substitution at L135 (EU Numbering).

269. The Fab of claim 267, wherein:
the HC CH1 substitution at F170 (EU Numbering) is an F170I, or F170V substitution,
the HC CH1 substitution at S183 (EU Numbering) is a S183L or S183I substitution,
the HC CH1 substitution at V185 (EU Numbering) is a V185L substitution, and/or
the LC CL1 substitution at L135 (EU Numbering) is a L135F substitution.

270. The Fab of claim 267, wherein the HC CH1 comprises an F170V, S183I and V185L (EU Numbering) substitution, and the LC CL comprises a L135F (EU Numbering) substitution.

271. The Fab of claim 267, wherein the HC CH1 comprises an F170I, S183L, and V185L (EU Numbering) substitution, and the LC CL comprises a L135F (EU Numbering) substitution.

272. An antibody comprising the Fab of claim 267.

273. The antibody of claim 272, further comprising a second Fab, wherein one but not both of the Fabs comprise an HC CH1 comprising a substitution at F126 and C220 (EU Numbering), and a LC CL comprising a substitution at E124 and C214 (lambda) or Q124 and C214 (kappa) (EU Numbering).

274. The antibody of claim 273, wherein the HC CH1 substitution at F126 (EU Numbering) is an F126C substitution, and the LC CL substitution at position 124 (EU Numbering) is an E124C (lambda) or Q124C (kappa) substitution.

275. The antibody of claim 273, wherein:
the HC CH1 substitution at C220 (EU Numbering) is a C220S substitution, and/or
the LC CL substitution at C214 (EU Numbering) is a C214S substitution.

276. An antibody comprising one or more IgG Fabs having a wild-type CH and a wild-type CL, and one or more Fabs of claim 267.

277. An antibody or a fragment thereof comprising:
a first heavy chain (HC1), a second heavy chain (HC2), a first light chain (LC1),
and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG,
wherein the HC1 CH1 comprises a substitution at F170, S183, and V185 (EU Numbering), and the LC1 CL comprises a substitution at L135 (EU Numbering), wherein the HC1 CH1 does not include any substitution selected from L128F, A141M, A141T, A141I, F170M, F170Y, F170S, F170A, S181I, S181T, S181M, S183A, S183V, and V185A (EU Numbering),
and wherein the LC1 CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (EU Numbering).

278. The antibody or fragment thereof of claim 277, wherein the HC1 CH1 consists of substitutions at F170, S183 and V185 (EU Numbering), and wherein the LC1 CL consists of a substitution at L135 (EU Numbering).

279. The antibody or fragment of claim 277, wherein the HC2 CH1 comprises SEQ ID NO: 9, and the LC2 CL comprises SEQ ID NO: 1 or 6.

280. The antibody or fragment thereof of claim 277, wherein the HC1 CH1 substitution at F170 (EU Numbering) is an F170I, or F170V substitution.

281. The antibody or fragment thereof of claim 277, wherein the HC1 CH1 substitution at S183 (EU Numbering) is a S183L or S183I substitution.

282. The antibody or fragment thereof of claim 277, wherein the HC1 CH1 substitution at V185 (EU Numbering) is a V185L substitution.

283. The antibody or fragment thereof of claim 277, wherein the LC1 CL1 substitution at L135 (EU Numbering) is a L135F substitution.

284. The antibody or fragment thereof of claim 277, wherein the HC1 CH1 comprises an F170V, S1831 and V185L (EU Numbering) substitution, and the LC1 CL comprises a L135F (EU Numbering) substitution.

285. The antibody or fragment thereof of claim 277, wherein the HC1 CH1 comprises an F170I, S183L, and V185L (EU Numbering) substitution, and the LC1 CL comprises a L135F (EU Numbering) substitution.

286. An antibody or a fragment thereof comprising:
a first heavy chain (HC1), a second heavy chain (HC2), a first light chain (LC1),
and second (LC2) light chain, wherein each HC1 and HC2 comprises a heavy chain variable domain (VH) and a first heavy chain constant domain (CH1) of a human IgG, and each LC1 and LC2 comprises a light chain variable domain (VL) and a light chain constant domain (CL) of a human IgG,
wherein the HC1 CH1 comprises a substitution at F126, F176, S189, V191, and C220 (EU Numbering), and the LC1 CL comprises a substitution at E124, L135, and C214 (lambda) or Q124, L135, and C214 (kappa) (EU Numbering),
wherein the HC1 CH1 does not include any substitution selected from L128F, A141M, A141T, A141I, F170M, F170Y, F170S, F170A, S181I, S181T, S181M, S183A, S183V, and V185A (EU Numbering), and wherein the LC1 CL does not include any substitution selected from F116A, F118V, S131T, S131D, V133A, V133I, L135Y, L135V, S162A, S162M, T164S, S174A, S176M, S176A, S176T, S176F, T178L, T178V, and T178I (EU Numbering).
